SEQUENCE LISTING (213 sequences)

SEQ ID NO: 1 - DNA, 21 bp, Artificial Sequence
  Description of Artificial Sequence Synthetic ACTB Forward Primer
gcaccacaccttctacaatga

SEQ ID NO: 2 - DNA, 20 bp, Artificial Sequence
  Description of Artificial Sequence Synthetic ACTB Reverse Primer
gtcatcttctcgcggttggc

SEQ ID NO: 3 - DNA, 20 bp, Artificial Sequence
  Description of Artificial Sequence Synthetic HPV L1 Primer
gcmcagggwcataayaatgg

SEQ ID NO: 4 - DNA, 24 bp, Artificial Sequence
  Description of Artificial Sequence Synthetic HPV L2 Primer
aataaactgtaaatcatattcctc

SEQ ID NO: 5 - DNA, 25 bp, Artificial Sequence
  Description of Artificial Sequence Synthetic HPV GP6+ Primer
gaaaaataaactgtaaatcatattc

SEQ ID NO: 6 - DNA, 22 bp, Artificial Sequence
  Description of Artificial Sequence Synthetic HPV 6 Probe 1
gcatccgtaactacatcttcca

SEQ ID NO: 7 - DNA, 25 bp, Artificial Sequence
  Description of Artificial Sequence Synthetic HPV 6 Probe 2
tgtgcatccgtaactacatcttcca

SEQ ID NO: 8 - DNA, 21 bp, Artificial Sequence
  Description of Artificial Sequence Synthetic HPV 7 Probe 1
acaccaacaccatatgacaat

SEQ ID NO: 9 - DNA, 27 bp, Artificial Sequence
  Description of Artificial Sequence Synthetic HPV 7 Probe 2
cgcccacaccaacaccatatgacaata

SEQ ID NO: 10 - DNA, 17 bp, Artificial Sequence
  Description of Artificial Sequence Synthetic HPV 10 Probe 1
cctcccctgccactacg

SEQ ID NO: 11 - DNA, 23 bp, Artificial Sequence
  Description of Artificial Sequence Synthetic HPV 10 Probe 2
tctgagcctcccctgccactacg

SEQ ID NO: 12 - DNA, 18 bp, Artificial Sequence
  Description of Artificial Sequence Synthetic HPV 11 Probe 1
atttgctggggaaaccac

SEQ ID NO: 13 - DNA, 20 bp, Artificial Sequence
  Description of Artificial Sequence Synthetic HPV 11 Probe 2
tatttgctggggaaaccact

SEQ ID NO: 14 - DNA, 22 bp, Artificial Sequence
  Description of Artificial Sequence Synthetic HPV 16 Probe 1
tgccatatctacttcagaaact

SEQ ID NO: 15 - DNA, 27 bp, Artificial Sequence
  Description of Artificial Sequence Synthetic HPV 16 Probe 2
tgtgctgccatatctacttcagaaact

SEQ ID NO: 16 - DNA, 21 bp, Artificial Sequence
  Description of Artificial Sequence Synthetic HPV 18 Probe 1
tctacacagtctccgtacctg

SEQ ID NO: 17 - DNA, 27 bp, Artificial Sequence
  Description of Artificial Sequence Synthetic HPV 18 Probe 2
aatatgtctacacagtctccgtacctg

SEQ ID NO: 18 - DNA, 22 bp, Artificial Sequence
  Description of Artificial Sequence Synthetic HPV 26 Probe 1
attatctgcagcatctgcatcc

SEQ ID NO: 19 - DNA, 28 bp, Artificial Sequence
  Description of Artificial Sequence Synthetic HPV 26 Probe 2
tagtacattatctgcagcatctgcatcc

SEQ ID NO: 20 - DNA, 26 bp, Artificial Sequence
  Description of Artificial Sequence Synthetic HPV 27 Probe 1
cagctgaggtgtctgataatactaat

SEQ ID NO: 21 - DNA, 31 bp, Artificial Sequence
  Description of Artificial Sequence Synthetic HPV 27 Probe 2
gtgtgcagctgaggtgtctgataatactaat

SEQ ID NO: 22 - DNA, 20 bp, Artificial Sequence
  Description of Artificial Sequence Synthetic HPV 30 Probe 1
aaccacacaaacgttatcca

SEQ ID NO: 23 - DNA, 26 bp, Artificial Sequence
  Description of Artificial Sequence Synthetic HPV 30 Probe 2
atctgcaaccacacaaacgttatcca

SEQ ID NO: 24 - DNA, 22 bp, Artificial Sequence
  Description of Artificial Sequence Synthetic HPV 31 Probe 1
ctgcaattgcaaacagtgatac

SEQ ID NO: 25 - DNA, 28 bp, Artificial Sequence
  Description of Artificial Sequence Synthetic HPV 31 Probe 2
tttgtgctgcaattgcaaacagtgatac

SEQ ID NO: 26 - DNA, 25 bp, Artificial Sequence
  Description of Artificial Sequence Synthetic HPV 32 Probe 1
gacacatacaagtctactaacttta

SEQ ID NO: 27 - DNA, 31 bp, Artificial Sequence
  Description of Artificial Sequence Synthetic HPV 32 Probe 2
actgaagacacatacaagtctactaacttta

SEQ ID NO: 28 - DNA, 25 bp, Artificial Sequence
  Description of Artificial Sequence Synthetic HPV 33  Probe 1
gcacacaagtaactagtgacagtac

SEQ ID NO: 29 - DNA, 31 bp, Artificial Sequence
  Description of Artificial Sequence Synthetic HPV 33  Probe 2
ctttatgcacacaagtaactagtgacagtac

SEQ ID NO: 30 - DNA, 19 bp, Artificial Sequence
  Description of Artificial Sequence Synthetic HPV 34 Probe 1
ccacaagtacaactgcacc

SEQ ID NO: 31 - DNA, 23 bp, Artificial Sequence
  Description of Artificial Sequence Synthetic HPV 34  Probe 2
caatccacaagtacaactgcacc

SEQ ID NO: 32 - DNA, 25 bp, Artificial Sequence
  Description of Artificial Sequence Synthetic HPV 35 Probe 1
tctgctgtgtcttctagtgacagta

SEQ ID NO: 33 - DNA, 30 bp, Artificial Sequence
  Description of Artificial Sequence Synthetic HPV 35  Probe 2
tgtgttctgctgtgtcttctagtgacagta

SEQ ID NO: 34 - DNA, 29 bp, Artificial Sequence
  Description of Artificial Sequence Synthetic HPV 39 Probe 1
acctctatagagtcttccataccttctac

SEQ ID NO: 35 - DNA, 35 bp, Artificial Sequence
  Description of Artificial Sequence Synthetic HPV 39 Probe 2
ttatctacctctatagagtcttccataccttctac

SEQ ID NO: 36 - DNA, 16 bp, Artificial Sequence
  Description of Artificial Sequence Synthetic HPV 40 Probe 1
agtcccccacaccaac

SEQ ID NO: 37 - DNA, 22 bp, Artificial Sequence
  Description of Artificial Sequence Synthetic HPV 40 Probe 2
ccacacagtcccccacaccaac

SEQ ID NO: 38 - DNA, 18 bp, Artificial Sequence
  Description of Artificial Sequence Synthetic HPV 42 Probe 1
cactgcaacatctggtga

SEQ ID NO: 39 - DNA, 24 bp, Artificial Sequence
  Description of Artificial Sequence Synthetic HPV 42 Probe 2
gtgtgccactgcaacatctggtga

SEQ ID NO: 40 - DNA, 22 bp, Artificial Sequence
  Description of Artificial Sequence Synthetic HPV 43 Probe 1
gcccagtacatatgacaatgca

SEQ ID NO: 41 - DNA, 28 bp, Artificial Sequence
  Description of Artificial Sequence Synthetic HPV 43 Probe 2
tactgtgcccagtacatatgacaatgca

SEQ ID NO: 42 - DNA, 18 bp, Artificial Sequence
  Description of Artificial Sequence Synthetic HPV 44 Probe 1
tacacagtcccctccgtc

SEQ ID NO: 43 - DNA, 24 bp, Artificial Sequence
  Description of Artificial Sequence Synthetic HPV 44 Probe 2
tgccactacacagtcccctccgtc

SEQ ID NO: 44 - DNA, 19 bp, Artificial Sequence
  Description of Artificial Sequence Synthetic HPV 45 Probe 1
cacaaaatcctgtgccaag

SEQ ID NO: 45 - DNA, 25 bp, Artificial Sequence
  Description of Artificial Sequence Synthetic HPV 45 Probe 2
cctctacacaaaatcctgtgccaag

SEQ ID NO: 46 - DNA, 20 bp, Artificial Sequence
  Description of Artificial Sequence Synthetic HPV 51 Probe 1
ggtttccccaacatttactc

SEQ ID NO: 47 - DNA, 23 bp, Artificial Sequence
  Description of Artificial Sequence Synthetic HPV 51 Probe 2
tgcggtttccccaacatttactc

SEQ ID NO: 48 - DNA, 22 bp, Artificial Sequence
  Description of Artificial Sequence Synthetic HPV 52 Probe 1
gctgaggttaaaaaggaaagca

SEQ ID NO: 49 - DNA, 30 bp, Artificial Sequence
  Description of Artificial Sequence Synthetic HPV 52 Probe 2
ctttatgtgctgaggttaaaaaggaaagca

SEQ ID NO: 50 - DNA, 23 bp, Artificial Sequence
  Description of Artificial Sequence Synthetic HPV 53 Probe 1
cgcaaccacacagtctatgtcta

SEQ ID NO: 51 - DNA, 30 bp, Artificial Sequence
  Description of Artificial Sequence Synthetic HPV 53 Probe 2
ctctttccgcaaccacacagtctatgtcta

SEQ ID NO: 52 - DNA, 17 bp, Artificial Sequence
  Description of Artificial Sequence Synthetic HPV 54 Probe 1
tacagcatccacgcagg

SEQ ID NO: 53 - DNA, 23 bp, Artificial Sequence
  Description of Artificial Sequence Synthetic HPV 54 Probe 2
gtgtgctacagcatccacgcagg

SEQ ID NO: 54 - DNA, 24 bp, Artificial Sequence
  Description of Artificial Sequence Synthetic HPV 55 Probe 1
ctacaactcagtctccatctacaa

SEQ ID NO: 55 - DNA, 30 bp, Artificial Sequence
  Description of Artificial Sequence Synthetic HPV 55 Probe 2
gtgctgctacaactcagtctccatctacaa

SEQ ID NO: 56 - DNA, 34 bp, Artificial Sequence
  Description of Artificial Sequence Synthetic HPV 56 Probe 1
gactattagtactgctacagaacagttaagtaaa

SEQ ID NO: 57 - DNA, 25 bp, Artificial Sequence
  Description of Artificial Sequence Synthetic HPV 56 Probe 2
tactgctacagaacagttaagtaaa

SEQ ID NO: 58 - DNA, 24 bp, Artificial Sequence
  Description of Artificial Sequence Synthetic HPV 57 Probe 1
ccactgtaaccacagaaactaatt

SEQ ID NO: 59 - DNA, 29 bp, Artificial Sequence
  Description of Artificial Sequence Synthetic HPV 57 Probe 2
gtgtgccactgtaaccacagaaactaatt

SEQ ID NO: 60 - DNA, 23 bp, Artificial Sequence
  Description of Artificial Sequence Synthetic HPV 58 Probe 1
tgcactgaagtaactaaggaagg

SEQ ID NO: 61 - DNA, 30 bp, Artificial Sequence
  Description of Artificial Sequence Synthetic HPV 58 Probe 2
gacattatgcactgaagtaactaaggaagg

SEQ ID NO: 62 - DNA, 29 bp, Artificial Sequence
  Description of Artificial Sequence Synthetic HPV 59 Probe 1
tctattcctaatgtatacacacctaccag

SEQ ID NO: 63 - DNA, 34 bp, Artificial Sequence
  Description of Artificial Sequence Synthetic HPV 59 Probe 2
cttcttctattcctaatgtatacacacctaccag

SEQ ID NO: 64 - DNA, 20 bp, Artificial Sequence
  Description of Artificial Sequence Synthetic HPV 61 Probe 1
tgctacatccccccctgtat

SEQ ID NO: 65 - DNA, 27 bp, Artificial Sequence
  Description of Artificial Sequence Synthetic HPV 61 Probe 2
tttgtactgctacatccccccctgtat

SEQ ID NO: 66 - DNA, 20 bp, Artificial Sequence
  Description of Artificial Sequence Synthetic HPV 62 Probe 1
actatttgtaccgcctccac

SEQ ID NO: 67 - DNA, 25 bp, Artificial Sequence
  Description of Artificial Sequence Synthetic HPV 62 Probe 2
actatttgtaccgcctccactgctg

SEQ ID NO: 68 - DNA, 26 bp, Artificial Sequence
  Description of Artificial Sequence Synthetic HPV 66 Probe 1
aatgcagctaaaagcacattaactaa

SEQ ID NO: 69 - DNA, 31 bp, Artificial Sequence
  Description of Artificial Sequence Synthetic HPV 66 Probe 2
ctattaatgcagctaaaagcacattaactaa

SEQ ID NO: 70 - DNA, 23 bp, Artificial Sequence
  Description of Artificial Sequence Synthetic HPV 67 Probe 1
aaaatcagaggctacatacaaaa

SEQ ID NO: 71 - DNA, 30 bp, Artificial Sequence
  Description of Artificial Sequence Synthetic HPV 67 Probe 2
ctgaggaaaaatcagaggctacatacaaaa

SEQ ID NO: 72 - DNA, 31 bp, Artificial Sequence
  Description of Artificial Sequence Synthetic HPV 68b Probe 1
ctactactactgaatcagctgtaccaaatat

SEQ ID NO: 73 - DNA, 36 bp, Artificial Sequence
  Description of Artificial Sequence Synthetic HPV 68b Probe 2
tttgtctactactactgaatcagctgtaccaaatat

SEQ ID NO: 74 - DNA, 21 bp, Artificial Sequence
  Description of Artificial Sequence Synthetic HPV 68a Probe 1
cagactctactgtaccagctg

SEQ ID NO: 75 - DNA, 23 bp, Artificial Sequence
  Description of Artificial Sequence Synthetic HPV 68a Probe 2
tacagactctactgtaccagctg

SEQ ID NO: 76 - DNA, 26 bp, Artificial Sequence
  Description of Artificial Sequence Synthetic HPV 68a Probe 3
tactacagactctactgtaccagctg

SEQ ID NO: 77 - DNA, 23 bp, Artificial Sequence
  Description of Artificial Sequence Synthetic HPV 68a Probe 4
cagactctactgtaccagctgtg

SEQ ID NO: 78 - DNA, 25 bp, Artificial Sequence
  Description of Artificial Sequence Synthetic HPV 69 Probe 1
cacaatctgcatctgccacttttaa

SEQ ID NO: 79 - DNA, 32 bp, Artificial Sequence
  Description of Artificial Sequence Synthetic HPV 69 Probe 2
gtatctgcacaatctgcatctgccacttttaa

SEQ ID NO: 80 - DNA, 17 bp, Artificial Sequence
  Description of Artificial Sequence Synthetic HPV 70 Probe 1
ccgaaacggccatacct

SEQ ID NO: 81 - DNA, 22 bp, Artificial Sequence
  Description of Artificial Sequence Synthetic HPV 70 Probe 2
ctgcaccgaaacggccatacct

SEQ ID NO: 82 - DNA, 21 bp, Artificial Sequence
  Description of Artificial Sequence Synthetic HPV 72 Probe 1
cacagcgtcctctgtatcaga

SEQ ID NO: 83 - DNA, 27 bp, Artificial Sequence
  Description of Artificial Sequence Synthetic HPV 72 Probe 2
tactgccacagcgtcctctgtatcaga

SEQ ID NO: 84 - DNA, 27 bp, Artificial Sequence
  Description of Artificial Sequence Synthetic HPV 73 Probe 1
aggtacacaggctagtagctctactac

SEQ ID NO: 85 - DNA, 30 bp, Artificial Sequence
  Description of Artificial Sequence Synthetic HPV 73 Probe 2
tgtaggtacacaggctagtagctctactac

SEQ ID NO: 86 - DNA, 20 bp, Artificial Sequence
  Description of Artificial Sequence Synthetic HPV 81 Probe 1
gctacatctgctgctgcaga

SEQ ID NO: 87 - DNA, 28 bp, Artificial Sequence
  Description of Artificial Sequence Synthetic HPV 81 Probe 2
tttgcacagctacatctgctgctgcaga

SEQ ID NO: 88 - DNA, 19 bp, Artificial Sequence
  Description of Artificial Sequence Synthetic HPV 82 Probe 1
ctccagcaaactttaagca

SEQ ID NO: 89 - DNA, 24 bp, Artificial Sequence
  Description of Artificial Sequence Synthetic HPV 82 Probe 2
ctccagcaaactttaagcaataca

SEQ ID NO: 90 - DNA, 21 bp, Artificial Sequence
  Description of Artificial Sequence Synthetic HPV 83 Probe 1
tgctgctacacaggctaatga

SEQ ID NO: 91 - DNA, 26 bp, Artificial Sequence
  Description of Artificial Sequence Synthetic HPV 83 Probe 2
tcagctgctgctacacaggctaatga

SEQ ID NO: 92 - DNA, 27 bp, Artificial Sequence
  Description of Artificial Sequence Synthetic HPV 84 Probe 1
accgaatcagaatataaacctaccaat

SEQ ID NO: 93 - DNA, 31 bp, Artificial Sequence
  Description of Artificial Sequence Synthetic HPV 84 Probe 2
caacaccgaatcagaatataaacctaccaat

SEQ ID NO: 94 - DNA, 23 bp, Artificial Sequence
  Description of Artificial Sequence Synthetic HPV 90 Probe 1
acaaacaccctctgacacataca

SEQ ID NO: 95 - DNA, 27 bp, Artificial Sequence
  Description of Artificial Sequence Synthetic HPV 90 Probe 2
ccacacaaacaccctctgacacataca

SEQ ID NO: 96 - DNA, 28 bp, Artificial Sequence
  Description of Artificial Sequence Synthetic HPV 91 Probe 1
tctgtgctacctactacatatgacaaca

SEQ ID NO: 97 - DNA, 34 bp, Artificial Sequence
  Description of Artificial Sequence Synthetic HPV 91 Probe 2
actgagtctgtgctacctactacatatgacaaca

SEQ ID NO: 98 - DNA, 30 bp, Artificial Sequence
  Description of Artificial Sequence Synthetic HPV Universal Probe 1
ttgttgggdtaatcagttgtttgttactgt

SEQ ID NO: 99 - DNA, 32 bp, Artificial Sequence
  Description of Artificial Sequence Synthetic HPV Universal Probe 2
tttgttactgttgtagatactactcgcagtac

SEQ ID NO: 100 - DNA, 30 bp, Artificial Sequence
  Description of Artificial Sequence Synthetic HPV Universal Probe 3
ttgttgggdtaatcarttrtttgttacdgt

SEQ ID NO: 101 - DNA, 23 bp, Artificial Sequence
  Description of Artificial Sequence Synthetic HPV Universal Probe 4
tttkttachgtkgtdgatacyac

SEQ ID NO: 102 - DNA, 25 bp, Artificial Sequence
  Description of Artificial Sequence Synthetic HPV Universal Probe 5
tgtttrttactgttgtdgayacyac

SEQ ID NO: 103 - DNA, 25 bp, Artificial Sequence
  Description of Artificial Sequence Synthetic HPV Universal Probe 6
tatttgtaactgttgtggataccac

SEQ ID NO: 104 - DNA, 23 bp, Artificial Sequence
  Description of Artificial Sequence Synthetic HPV Universal Probe 7
tttrttactgttgtdgayacyac

SEQ ID NO: 105 - DNA, 25 bp, Artificial Sequence
  Description of Artificial Sequence Synthetic HPV Universal Probe 8
tatttrttactgttgtdgayacyac

SEQ ID NO: 106 - DNA, 22 bp, Artificial Sequence
  Description of Artificial Sequence Synthetic ACTB-1 Probe
accccgtgctgctgaccgaggc

SEQ ID NO: 107 - DNA, 19 bp, Artificial Sequence
  Description of Artificial Sequence Synthetic ACTB-2 Probe
caccccgtgctgctgaccg

SEQ ID NO: 108 - DNA, 23 bp, Artificial Sequence
  Description of Artificial Sequence Synthetic ACTB-3 Probe
caccccgtgctgctgaccgaggc

SEQ ID NO: 109 - DNA, 20 bp, Artificial Sequence
  Description of Artificial Sequence Synthetic ACTB-4 Probe
gctgcgtgtggctcccgagg

SEQ ID NO: 110 - DNA, 47 bp, Artificial Sequence
  Description of Artificial Sequence Synthetic HPV 6 d type Probe 1
ccctaaccctaanttagggttaggggcatccgtaactacatcttcca

SEQ ID NO: 111 - DNA, 50 bp, Artificial Sequence
  Description of Articial Sequence Synthetic HPV 6 d type Probe 2
ccctaaccctaanttagggttagggtgtgcatccgtaactacatcttcca

SEQ ID NO: 112 - DNA, 46 bp, Artificial Sequence
  Description of Articial Sequence Synthetic HPV 7 d type Probe 1
ccctaaccctaanttagggttagggacaccaacaccatatgacaat

SEQ ID NO: 113 - DNA, 52 bp, Artificial Sequence
  Description of Articial Sequence Synthetic HPV 7 d type Probe 2
ccctaaccctaanttagggttagggcgcccacaccaacaccatatgacaata

SEQ ID NO: 114 - DNA, 42 bp, Artificial Sequence
  Description of Articial Sequence Synthetic HPV 10 d type Probe 1
ccctaaccctaanttagggttagggcctcccctgccactacg

SEQ ID NO: 115 - DNA, 48 bp, Artificial Sequence
  Description of Articial Sequence Synthetic HPV 10 d type  Probe 2
ccctaaccctaanttagggttagggtctgagcctcccctgccactacg

SEQ ID NO: 116 - DNA, 43 bp, Artificial Sequence
  Description of Articial Sequence Synthetic HPV 11 d type Probe 1
ccctaaccctaanttagggttagggatttgctggggaaaccac

SEQ ID NO: 117 - DNA, 45 bp, Artificial Sequence
  Description of Articial Sequence Synthetic HPV 11 d type Probe 2
ccctaaccctaanttagggttagggtatttgctggggaaaccact

SEQ ID NO: 118 - DNA, 47 bp, Artificial Sequence
  Description of Articial Sequence Synthetic HPV 16 d type Probe 1
ccctaaccctaanttagggttagggtgccatatctacttcagaaact

SEQ ID NO: 119 - DNA, 52 bp, Artificial Sequence
  Description of Articial Sequence Synthetic HPV 16 d type Probe 2
ccctaaccctaanttagggttagggtgtgctgccatatctacttcagaaact

SEQ ID NO: 120 - DNA, 46 bp, Artificial Sequence
  Description of Articial Sequence Synthetic HPV 18 d type Probe 1
ccctaaccctaanttagggttagggtctacacagtctccgtacctg

SEQ ID NO: 121 - DNA, 52 bp, Artificial Sequence
  Description of Articial Sequence Synthetic HPV 18 d type Probe 2
ccctaaccctaanttagggttagggaatatgtctacacagtctccgtacctg

SEQ ID NO: 122 - DNA, 47 bp, Artificial Sequence
  Description of Articial Sequence Synthetic HPV 26 d type Probe 1
ccctaaccctaanttagggttagggattatctgcagcatctgcatcc

SEQ ID NO: 123 - DNA, 53 bp, Artificial Sequence
  Description of Articial Sequence Synthetic HPV 26 d type Probe 2
ccctaaccctaanttagggttagggtagtacattatctgcagcatctgcatcc

SEQ ID NO: 124 - DNA, 51 bp, Artificial Sequence
  Description of Articial Sequence Synthetic HPV 27 d type Probe 1
ccctaaccctaanttagggttagggcagctgaggtgtctgataatactaat

SEQ ID NO: 125 - DNA, 56 bp, Artificial Sequence
  Description of Articial Sequence Synthetic HPV 27 d type Probe 2
ccctaaccctaanttagggttaggggtgtgcagctgaggtgtctgataatactaat

SEQ ID NO: 126 - DNA, 45 bp, Artificial Sequence
  Description of Articial Sequence Synthetic HPV 30 d type Probe 1
ccctaaccctaanttagggttagggaaccacacaaacgttatcca

SEQ ID NO: 127 - DNA, 51 bp, Artificial Sequence
  Description of Articial Sequence Synthetic HPV 30 d type Probe 2
ccctaaccctaanttagggttagggatctgcaaccacacaaacgttatcca

SEQ ID NO: 128 - DNA, 47 bp, Artificial Sequence
  Description of Articial Sequence Synthetic HPV 31 d type Probe 1
ccctaaccctaanttagggttagggctgcaattgcaaacagtgatac

SEQ ID NO: 129 - DNA, 53 bp, Artificial Sequence
  Description of Articial Sequence Synthetic HPV 31 d type Probe 2
ccctaaccctaanttagggttagggtttgtgctgcaattgcaaacagtgatac

SEQ ID NO: 130 - DNA, 50 bp, Artificial Sequence
  Description of Articial Sequence Synthetic HPV 32 d type Probe 1
ccctaaccctaanttagggttaggggacacatacaagtctactaacttta

SEQ ID NO: 131 - DNA, 56 bp, Artificial Sequence
  Description of Articial Sequence Synthetic HPV 32 d type Probe 2
ccctaaccctaanttagggttagggactgaagacacatacaagtctactaacttta

SEQ ID NO: 132 - DNA, 50 bp, Artificial Sequence
  Description of Articial Sequence Synthetic HPV 33  d type Probe 1
ccctaaccctaanttagggttaggggcacacaagtaactagtgacagtac

SEQ ID NO: 133 - DNA, 56 bp, Artificial Sequence
  Description of Articial Sequence Synthetic HPV 33  d type Probe 2
ccctaaccctaanttagggttagggctttatgcacacaagtaactagtgacagtac

SEQ ID NO: 134 - DNA, 44 bp, Artificial Sequence
  Description of Articial Sequence Synthetic HPV 34  d type Probe 1
ccctaaccctaanttagggttagggccacaagtacaactgcacc

SEQ ID NO: 135 - DNA, 48 bp, Artificial Sequence
  Description of Articial Sequence Synthetic HPV 34  d type Probe 2
ccctaaccctaanttagggttagggcaatccacaagtacaactgcacc

SEQ ID NO: 136 - DNA, 50 bp, Artificial Sequence
  Description of Articial Sequence Synthetic HPV 35  d type Probe 1
ccctaaccctaanttagggttagggtctgctgtgtcttctagtgacagta

SEQ ID NO: 137 - DNA, 55 bp, Artificial Sequence
  Description of Articial Sequence Synthetic HPV 35  d type Probe 2
ccctaaccctaanttagggttagggtgtgttctgctgtgtcttctagtgacagta

SEQ ID NO: 138 - DNA, 54 bp, Artificial Sequence
  Description of Articial Sequence Synthetic HPV 39 d type Probe 1
ccctaaccctaanttagggttagggacctctatagagtcttccataccttctac

SEQ ID NO: 139 - DNA, 60 bp, Artificial Sequence
  Description of Articial Sequence Synthetic HPV 39 d type Probe 2
ccctaaccctaanttagggttagggttatctacctctatagagtcttccataccttctac

SEQ ID NO: 140 - DNA, 41 bp, Artificial Sequence
  Description of Articial Sequence Synthetic HPV 40 d type Probe 1
ccctaaccctaanttagggttagggagtcccccacaccaac

SEQ ID NO: 141 - DNA, 47 bp, Artificial Sequence
  Description of Articial Sequence Synthetic HPV 40 d type Probe 2
ccctaaccctaanttagggttagggccacacagtcccccacaccaac

SEQ ID NO: 142 - DNA, 43 bp, Artificial Sequence
  Description of Articial Sequence Synthetic HPV 42 d type Probe 1
ccctaaccctaanttagggttagggcactgcaacatctggtga

SEQ ID NO: 143 - DNA, 49 bp, Artificial Sequence
  Description of Articial Sequence Synthetic HPV 42 d type Probe 2
ccctaaccctaanttagggttaggggtgtgccactgcaacatctggtga

SEQ ID NO: 144 - DNA, 47 bp, Artificial Sequence
  Description of Articial Sequence Synthetic HPV 43 d type Probe 1
ccctaaccctaanttagggttaggggcccagtacatatgacaatgca

SEQ ID NO: 145 - DNA, 53 bp, Artificial Sequence
  Description of Articial Sequence Synthetic HPV 43 d type Probe 2
ccctaaccctaanttagggttagggtactgtgcccagtacatatgacaatgca

SEQ ID NO: 146 - DNA, 43 bp, Artificial Sequence
  Description of Articial Sequence Synthetic HPV 44 d type Probe 1
ccctaaccctaanttagggttagggtacacagtcccctccgtc

SEQ ID NO: 147 - DNA, 49 bp, Artificial Sequence
  Description of Articial Sequence Synthetic HPV 44 d type Probe 2
ccctaaccctaanttagggttagggtgccactacacagtcccctccgtc

SEQ ID NO: 148 - DNA, 44 bp, Artificial Sequence
  Description of Articial Sequence Synthetic HPV 45 d type Probe 1
ccctaaccctaanttagggttagggcacaaaatcctgtgccaag

SEQ ID NO: 149 - DNA, 50 bp, Artificial Sequence
  Description of Articial Sequence Synthetic HPV 45 d type Probe 2
ccctaaccctaanttagggttagggcctctacacaaaatcctgtgccaag

SEQ ID NO: 150 - DNA, 45 bp, Artificial Sequence
  Description of Articial Sequence Synthetic HPV 51 d type Probe 1
ccctaaccctaanttagggttagggggtttccccaacatttactc

SEQ ID NO: 151 - DNA, 48 bp, Artificial Sequence
  Description of Articial Sequence Synthetic HPV 51 d type Probe 2
ccctaaccctaanttagggttagggtgcggtttccccaacatttactc

SEQ ID NO: 152 - DNA, 47 bp, Artificial Sequence
  Description of Articial Sequence Synthetic HPV 52 d type Probe 1
ccctaaccctaanttagggttaggggctgaggttaaaaaggaaagca

SEQ ID NO: 153 - DNA, 55 bp, Artificial Sequence
  Description of Articial Sequence Synthetic HPV 52 d type Probe 2
ccctaaccctaanttagggttagggctttatgtgctgaggttaaaaaggaaagca

SEQ ID NO: 154 - DNA, 48 bp, Artificial Sequence
  Description of Articial Sequence Synthetic HPV 53 d type Probe 1
ccctaaccctaanttagggttagggcgcaaccacacagtctatgtcta

SEQ ID NO: 155 - DNA, 55 bp, Artificial Sequence
  Description of Articial Sequence Synthetic HPV 53 d type Probe 2
ccctaaccctaanttagggttagggctctttccgcaaccacacagtctatgtcta

SEQ ID NO: 156 - DNA, 42 bp, Artificial Sequence
  Description of Articial Sequence Synthetic HPV 54 d type Probe 1
ccctaaccctaanttagggttagggtacagcatccacgcagg

SEQ ID NO: 157 - DNA, 48 bp, Artificial Sequence
  Description of Articial Sequence Synthetic HPV 54 d type Probe 2
ccctaaccctaanttagggttaggggtgtgctacagcatccacgcagg

SEQ ID NO: 158 - DNA, 49 bp, Artificial Sequence
  Description of Articial Sequence Synthetic HPV 55 d type Probe 1
ccctaaccctaanttagggttagggctacaactcagtctccatctacaa

SEQ ID NO: 159 - DNA, 55 bp, Artificial Sequence
  Description of Articial Sequence Synthetic HPV 55 d type Probe 2
ccctaaccctaanttagggttaggggtgctgctacaactcagtctccatctacaa

SEQ ID NO: 160 - DNA, 59 bp, Artificial Sequence
  Description of Articial Sequence Synthetic HPV 56 d type Probe 1
ccctaaccctaanttagggttaggggactattagtactgctacagaacagttaagtaaa

SEQ ID NO: 161 - DNA, 50 bp, Artificial Sequence
  Description of Articial Sequence Synthetic HPV 56 d type Probe 2
ccctaaccctaanttagggttagggtactgctacagaacagttaagtaaa

SEQ ID NO: 162 - DNA, 49 bp, Artificial Sequence
  Description of Articial Sequence Synthetic HPV 57 d type Probe 1
ccctaaccctaanttagggttagggccactgtaaccacagaaactaatt

SEQ ID NO: 163 - DNA, 54 bp, Artificial Sequence
  Description of Articial Sequence Synthetic HPV 57 d type Probe 2
ccctaaccctaanttagggttaggggtgtgccactgtaaccacagaaactaatt

SEQ ID NO: 164 - DNA, 48 bp, Artificial Sequence
  Description of Articial Sequence Synthetic HPV 58 d type Probe 1
ccctaaccctaanttagggttagggtgcactgaagtaactaaggaagg

SEQ ID NO: 165 - DNA, 55 bp, Artificial Sequence
  Description of Articial Sequence Synthetic HPV 58 d type Probe 2
ccctaaccctaanttagggttaggggacattatgcactgaagtaactaaggaagg

SEQ ID NO: 166 - DNA, 54 bp, Artificial Sequence
  Description of Articial Sequence Synthetic HPV 59 d type Probe 1
ccctaaccctaanttagggttagggtctattcctaatgtatacacacctaccag

SEQ ID NO: 167 - DNA, 59 bp, Artificial Sequence
  Description of Articial Sequence Synthetic HPV 59 d type Probe 2
ccctaaccctaanttagggttagggcttcttctattcctaatgtatacacacctaccag

SEQ ID NO: 168 - DNA, 45 bp, Artificial Sequence
  Description of Articial Sequence Synthetic HPV 61 d type Probe 1
ccctaaccctaanttagggttagggtgctacatccccccctgtat

SEQ ID NO: 169 - DNA, 52 bp, Artificial Sequence
  Description of Articial Sequence Synthetic HPV 61 d type Probe 2
ccctaaccctaanttagggttagggtttgtactgctacatccccccctgtat

SEQ ID NO: 170 - DNA, 45 bp, Artificial Sequence
  Description of Articial Sequence Synthetic HPV 62 d type Probe 1
ccctaaccctaanttagggttagggactatttgtaccgcctccac

SEQ ID NO: 171 - DNA, 50 bp, Artificial Sequence
  Description of Articial Sequence Synthetic HPV 62 d type Probe 2
ccctaaccctaanttagggttagggactatttgtaccgcctccactgctg

SEQ ID NO: 172 - DNA, 51 bp, Artificial Sequence
  Description of Articial Sequence Synthetic HPV 66 d type Probe 1
ccctaaccctaanttagggttagggaatgcagctaaaagcacattaactaa

SEQ ID NO: 173 - DNA, 56 bp, Artificial Sequence
  Description of Articial Sequence Synthetic HPV 66 d type Probe 2
ccctaaccctaanttagggttagggctattaatgcagctaaaagcacattaactaa

SEQ ID NO: 174 - DNA, 48 bp, Artificial Sequence
  Description of Articial Sequence Synthetic HPV 67 d type Probe 1
ccctaaccctaanttagggttagggaaaatcagaggctacatacaaaa

SEQ ID NO: 175 - DNA, 55 bp, Artificial Sequence
  Description of Articial Sequence Synthetic HPV 67 d type Probe 2
ccctaaccctaanttagggttagggctgaggaaaaatcagaggctacatacaaaa

SEQ ID NO: 176 - DNA, 56 bp, Artificial Sequence
  Description of Articial Sequence Synthetic HPV 68b d type Probe 1
ccctaaccctaanttagggttagggctactactactgaatcagctgtaccaaatat

SEQ ID NO: 177 - DNA, 61 bp, Artificial Sequence
  Description of Articial Sequence Synthetic HPV 68b d type Probe 2
ccctaaccctaanttagggttagggtttgtctactactactgaatcagctgtaccaaatat

SEQ ID NO: 178 - DNA, 46 bp, Artificial Sequence
  Description of Articial Sequence Synthetic HPV 68a d type Probe 1
ccctaaccctaanttagggttagggcagactctactgtaccagctg

SEQ ID NO: 179 - DNA, 48 bp, Artificial Sequence
  Description of Articial Sequence Synthetic HPV 68a d type Probe 2
ccctaaccctaanttagggttagggtacagactctactgtaccagctg

SEQ ID NO: 180 - DNA, 51 bp, Artificial Sequence
  Description of Articial Sequence Synthetic HPV 68a d type Probe 3
ccctaaccctaanttagggttagggtactacagactctactgtaccagctg

SEQ ID NO: 181 - DNA, 48 bp, Artificial Sequence
  Description of Articial Sequence Synthetic HPV 68a d type Probe 4
ccctaaccctaanttagggttagggcagactctactgtaccagctgtg

SEQ ID NO: 182 - DNA, 50 bp, Artificial Sequence
  Description of Articial Sequence Synthetic HPV 69 d type Probe 1
ccctaaccctaanttagggttagggcacaatctgcatctgccacttttaa

SEQ ID NO: 183 - DNA, 57 bp, Artificial Sequence
  Description of Articial Sequence Synthetic HPV 69 d type Probe 2
ccctaaccctaanttagggttaggggtatctgcacaatctgcatctgccacttttaa

SEQ ID NO: 184 - DNA, 42 bp, Artificial Sequence
  Description of Articial Sequence Synthetic HPV 70 d type Probe 1
ccctaaccctaanttagggttagggccgaaacggccatacct

SEQ ID NO: 185 - DNA, 47 bp, Artificial Sequence
  Description of Articial Sequence Synthetic HPV 70 d type Probe 2
ccctaaccctaanttagggttagggctgcaccgaaacggccatacct

SEQ ID NO: 186 - DNA, 46 bp, Artificial Sequence
  Description of Articial Sequence Synthetic HPV 72 d type Probe 1
ccctaaccctaanttagggttagggcacagcgtcctctgtatcaga

SEQ ID NO: 187 - DNA, 52 bp, Artificial Sequence
  Description of Articial Sequence Synthetic HPV 72 d type Probe 2
ccctaaccctaanttagggttagggtactgccacagcgtcctctgtatcaga

SEQ ID NO: 188 - DNA, 52 bp, Artificial Sequence
  Description of Articial Sequence Synthetic HPV 73 d type Probe 1
ccctaaccctaanttagggttagggaggtacacaggctagtagctctactac

SEQ ID NO: 189 - DNA, 55 bp, Artificial Sequence
  Description of Articial Sequence Synthetic HPV 73 d type Probe 2
ccctaaccctaanttagggttagggtgtaggtacacaggctagtagctctactac

SEQ ID NO: 190 - DNA, 45 bp, Artificial Sequence
  Description of Articial Sequence Synthetic HPV 81 d type Probe 1
ccctaaccctaanttagggttaggggctacatctgctgctgcaga

SEQ ID NO: 191 - DNA, 53 bp, Artificial Sequence
  Description of Articial Sequence Synthetic HPV 81 d type Probe 2
ccctaaccctaanttagggttagggtttgcacagctacatctgctgctgcaga

SEQ ID NO: 192 - DNA, 44 bp, Artificial Sequence
  Description of Articial Sequence Synthetic HPV 82 d type Probe 1
ccctaaccctaanttagggttagggctccagcaaactttaagca

SEQ ID NO: 193 - DNA, 49 bp, Artificial Sequence
  Description of Articial Sequence Synthetic HPV 82 d type Probe 2
ccctaaccctaanttagggttagggctccagcaaactttaagcaataca

SEQ ID NO: 194 - DNA, 46 bp, Artificial Sequence
  Description of Articial Sequence Synthetic HPV 83 d type Probe 1
ccctaaccctaanttagggttagggtgctgctacacaggctaatga

SEQ ID NO: 195 - DNA, 51 bp, Artificial Sequence
  Description of Articial Sequence Synthetic HPV 83 d type Probe 2
ccctaaccctaanttagggttagggtcagctgctgctacacaggctaatga

SEQ ID NO: 196 - DNA, 52 bp, Artificial Sequence
  Description of Articial Sequence Synthetic HPV 84 d type Probe 1
ccctaaccctaanttagggttagggaccgaatcagaatataaacctaccaat

SEQ ID NO: 197 - DNA, 56 bp, Artificial Sequence
  Description of Articial Sequence Synthetic HPV 84 d type Probe 2
ccctaaccctaanttagggttagggcaacaccgaatcagaatataaacctaccaat

SEQ ID NO: 198 - DNA, 48 bp, Artificial Sequence
  Description of Articial Sequence Synthetic HPV 90 d type Probe 1
ccctaaccctaanttagggttagggacaaacaccctctgacacataca

SEQ ID NO: 199 - DNA, 52 bp, Artificial Sequence
  Description of Articial Sequence Synthetic HPV 90 d type Probe 2
ccctaaccctaanttagggttagggccacacaaacaccctctgacacataca

SEQ ID NO: 200 - DNA, 53 bp, Artificial Sequence
  Description of Articial Sequence Synthetic HPV 91 d type Probe 1
ccctaaccctaanttagggttagggtctgtgctacctactacatatgacaaca

SEQ ID NO: 201 - DNA, 59 bp, Artificial Sequence
  Description of Articial Sequence Synthetic HPV 91 d type Probe 2
ccctaaccctaanttagggttagggactgagtctgtgctacctactacatatgacaaca

SEQ ID NO: 202 - DNA, 55 bp, Artificial Sequence
  Description of Articial Sequence Synthetic HPV Universal d type Probe 1
ccctaaccctaanttagggttagggttgttgggdtaatcagttgtttgttactgt

SEQ ID NO: 203 - DNA, 57 bp, Artificial Sequence
  Description of Articial Sequence Synthetic HPV Universal d type Probe 2
ccctaaccctaanttagggttagggtttgttactgttgtagatactactcgcagtac

SEQ ID NO: 204 - DNA, 55 bp, Artificial Sequence
  Description of Articial Sequence Synthetic HPV Universal d type Probe 3
ccctaaccctaanttagggttagggttgttgggdtaatcarttrtttgttacdgt

SEQ ID NO: 205 - DNA, 48 bp, Artificial Sequence
  Description of Articial Sequence Synthetic HPV Universal d type Probe 4
ccctaaccctaanttagggttagggtttkttachgtkgtdgatacyac

SEQ ID NO: 206 - DNA, 50 bp, Artificial Sequence
  Description of Articial Sequence Synthetic HPV Universal d type Probe 5
ccctaaccctaanttagggttagggtgtttrttactgttgtdgayacyac

SEQ ID NO: 207 - DNA, 50 bp, Artificial Sequence
  Description of Articial Sequence Synthetic HPV Universal d type Probe 6
ccctaaccctaanttagggttagggtatttgtaactgttgtggataccac

SEQ ID NO: 208 - DNA, 48 bp, Artificial Sequence
  Description of Articial Sequence Synthetic HPV Universal d type Probe 7
ccctaaccctaanttagggttagggtttrttactgttgtdgayacyac

SEQ ID NO: 209 - DNA, 50 bp, Artificial Sequence
  Description of Articial Sequence Synthetic HPV Universal d type Probe 8
ccctaaccctaanttagggttagggtatttrttactgttgtdgayacyac

SEQ ID NO: 210 - DNA, 47 bp, Artificial Sequence
  Description of Articial Sequence Synthetic ACTB-1 d type Probe
ccctaaccctaanttagggttagggaccccgtgctgctgaccgaggc

SEQ ID NO: 211 - DNA, 44 bp, Artificial Sequence
  Description of Articial Sequence Synthetic ACTB-2 d type Probe
ccctaaccctaanttagggttagggcaccccgtgctgctgaccg

SEQ ID NO: 212 - DNA, 48 bp, Artificial Sequence
  Description of Articial Sequence Synthetic ACTB-3 d type Probe
ccctaaccctaanttagggttagggcaccccgtgctgctgaccgaggc

SEQ ID NO: 213 - DNA, 45 bp, Artificial Sequence
  Description of Articial Sequence Synthetic ACTB-4 d type Probe
ccctaaccctaanttagggttaggggctgcgtgtggctcccgagg

(19) United States
(12) Patent Application Publication
(10) Pub. No.: US 2013/0184164 A1
(43) Pub. Date: Jul. 18, 2013

(54) DNA Chip for Genotyping of Human Papilloma Virus, Kit Having Same, and Method for Genotyping

(71) Applicants: Woo Chul Moon, Gangnam-gu (KR); Myung Ryurl Oh, Suwon-si (KR)

(72) Inventors: Woo Chul Moon, Gangnam-gu (KR); Myung Ryurl Oh, Suwon-si (KR)

(73) Assignee: GOODGENE, INC., Seoul (KR)

(21) Appl. No.: 13/704,942

(22) PCT Filed: Jun. 25, 2010
(86) PCT No.: PCT/KR2010/004164

§ 371 Date: Mar. 19, 2013

(30) Foreign Application Priority Data
Jun. 17, 2010 (KR) 10-2010-0057676

(51) Int. Cl. C12Q 1/70

(52) U.S. Cl. CPC C12Q 1/708
USPC 506/2; 506/16

(57) ABSTRACT

Disclosed is a DNA chip (or DNA microarray) on which probes complementarily binding to the nucleic acids of 44 types of HPV, which is the main cause of cervical cancer and the most common cause of sexually transmitted diseases, are spotted, a genotyping kit including same and a genotyping method using same. In accordance with the present disclosure, all the 44 types of HPV invading the genitalia can be detected and coinfection by more than one type of HPV can be diagnosed accurately. The sensitivity and specificity of HPV genotyping is close to 100% and a number of samples can be tested quickly. The present disclosure is very useful in predicting cervical cancer and precancerous lesions.

The present disclosure relates to a DNA chip for genotyping human papillomavirus (HPV), a kit including same and a method for genotyping HPV. More particularly, it relates to a DNA chip (or DNA microarray) on which probes complementarily binding to the nucleic acids of 44 types of HPV, which is the main cause of cervical cancer and the most common cause of sexually transmitted diseases, are spotted, a genotyping kit including same and a genotyping method using same.

Human papillomavirus (HPV) is a virus transmitted to humans through sexual contact and is very important in two aspects.

Firstly, HPV infection is the most common sexually transmitted infection in humans with the highest prevalent rate. In the US, HPV infection is found in 26.8% of women aged between 14 and 59 and it is thought that 80% of women are infected at least once. The infection occurs well particularly in sexually active, fertile women, and the prevalence is estimated to increase. Hence, periodic HPV testing is necessary for adult women and HPV testing is included in testing of sexually transmitted infections (U.S. Department of Health And Human Services, Centers for Disease Control and Prevention, National Center for HIV/AIDS, Viral Hepatitis, STD, and TB, Prevention Division of STD Prevention. Sexually Transmitted Disease Surveillance 2008. Division of STD Prevention. 2009: November; Tchernev G. Sexually transmitted papillomavirus infections: epidemiology pathogenesis, clinic, morphology, important differential diagnostic aspects, current diagnostic and treatment options. An Bras Dermatol. 2009; 84(4): 377-89).

Secondly, HPV is clearly proven to cause tumors and cancers in human. HPV, particularly the high-risk type HPV, is the cause of nearly all cases of cervical cancer. HPV infiltrates into the epithelium of human skin or mucous membranes, thereby causing inflammation and hyperproliferation. In most cases, the hyperproliferation is simply skin warts, genital or anal warts, or benign tumors such as condylomata acuminata. However, HPV can cause cancer and, indeed, almost all cervical cancers, most of oral cancers, pharyngeal cancers and laryngeal cancers and a number of anal cancers are caused by HPV. HPV is of great importance in that it can be fatal by causing cancer. Caners and precancerous lesions of the cervix, anus, etc. can be diagnosed early by HPV testing. Indeed, it is shown that HPV testing is superior in prediction sensitivity of cervical cancer than the Papanicolaou test, or Pap smear, which is the standard screening method for diagnosis of cervical cancer. Accordingly, it is approved as the cervical cancer screening test in several countries including the US (Howley P M. Virology. Vol 2, 1996, 2045-2109; Murinoz N et al., N Engl J Med, 2003, 348: 518-27; Parkin M, F. Bray F, J. Ferlay J and P. Pisani P. Global cancer statistics, 2002. C.A. Cancer J. Clin. 2005; National Network of STD/HIV Prevention Training Center. Genital human papillomavirus infection. February 2008). For these reasons, the HPV market is very large and the HPV testing is of great economic value.

Cervical cancer is the second most common cancer in women globally after breast cancer. It is also one of the main causes of cancer-related deaths of women in the developing countries. It is reported that about 440,000 new cases and 270,000 deaths occur each year worldwide. In particular, it is one of the main causes of female death in developing countries. In Korean women, cervical cancer (10.6%) ranks third in incidence following stomach cancer (15.8%) and breast cancer (15.1%). In recent years, human papillomavirus infection has significantly increased in young women of 20s and 30s, accounting for 32% of all sexually transmitted disease patients, and become a severe health concern. According to the 2002 Annual Report of the Korea Central Cancer Registry, Korea shows higher incidence rate with 3,979 cases in 2002 as compared to developed countries. Among the all malignant tumors occurring in women, cervical cancer ranks fifth with 9.1% after breast cancer, stomach cancer, colorectal cancer and thyroid cancer, with the highest incidence in 40s as 29.3%. According to the data from the Korea Central Cancer Registry, cervical cancer ranks 2nd when including carcinoma in situ of the cervix, which is a pre-cancer stage, and ranks 5th when excluding the carcinoma in situ. However, if cervical dysplasia not registered in the cancer statistics is also included, it is still the most important cancer in women. Formerly, about 90% of the cancer of uterine cancer was cervical cancer. But, recently, the incidence of uterine body cancer is increasing and that of cervical cancer is decreasing. Presently, the ratio of cervical cancer to uterine body cancer is about 5:1 (http://www.ncc.re.kr:9000/nciapps/user/basicinfo/each_info.jsp?grpcode=1H00).

Epidemiological studies about the cause of cervical cancer reveal that risk of cervical cancer is higher in women of low level of education or economy or poor hygiene, in women who started sexual intercourse in young ages, in women who have many childbirth experiences, in women who have promiscuous sex partners, and in women who are diagnosed positive in human papillomavirus testing. This suggests that cervical cancer is closely related with sexually transmitted infection and it is widely recognized that human papillomavirus is the major cause of cervical cancer (Jae Won Kim, Ju Won Roh, Moon Hong Kim, Noh Hyun Park, Polymorphisms in E7 Gene of Human Papillomavirus Type 16 Found in Cervical Tissues from Korean Women, J Korean Cancer Assoc. 2000; 32(5) 875-883).

At present, about 120 types of HPV are known based on subtypes or genotypes. Among them, 83 types are known about their base sequence and structure. About 40 types of HPV are the so-called anogenital type or genital HPV infecting the anogenital region, i.e. the skin and mucosa of the vagina, cervix, urethra and penis. While the majority of HPV infections cause no symptoms in most people, some types can cause warts. Others can lead to precancerous lesions such as high grade squamous intraepithelial lesion (HSIL) or cervical intraepithelial neoplasm, and some of them may develop into cancer. HPV types that can lead to precancerous lesions and cancer are called high-risk type HPV and others are called low-risk type HPV. Some researchers classify HPV into high-risk, moderate-risk and low-risk groups. High-risk type HPV includes HPV types 16, 18, 31, 33, 35, 39, 45, 51, 52, 56, 58, 59, 68 and 82. And, low-risk type HPV includes HPV type 6, 11, 34, 40, 42, 43, 44, 54, 55, 61, 62, 72 and 81. Probably high-risk type HPVs which are suspected of being high-risk but not identified yet include HPV types 26, 53, 66, 67, 69, 70 and 73. Besides, there are other types that are not clearly identified such as HPV types 7, 10, 27, 30, 32, 57, 83, 84 and 91. Globally, it is reported that 49.9% of cervical cancer patients are infected by HPV type 16, 13.7% by HPV type 18, 7.2% by HPV types 31, 33 and 35, and 8.4% by HPV type 45.

According to the Merck's report, HPV types 16 and 18 are of particular importance. These two types of HPV are reported to cause about 60-70% of cervical cancer, cervical intraepithelial neoplasm (CIN) and HSIL and HPV types 6 and 11 are known to cause about 90% of genital warts. However, there are differences in the epidemiology of HPV types in different races and countries. Indeed, as will be described later, the data from Korea have slight difference from those of other countries. Another report from Korea classifies HPV types 16 and 18 as high-risk HPVs causing cervical cancer, HPV types 31, 33, 35, 45 and 52 as moderate-risk HPVs, and HPV types 6 and 11 as low-risk HPVs and asserts that early screening or diagnosis of cervical cancer is possible through genotyping of HPV (Jae Won Kim, Ju Won Roh, Moon Hong Kim, Noh Hyun Park, Polymorphisms in E7 Gene of Human Papillomavirus Type 16 Found in Cervical Tissues from Korean Women, J Korean Cancer Assoc. 2000; 32(5) 875-883; (http://www.cmcbaoro.or.kr/guide/guide02_{—}02.jsp?dtno=209&dcno=411; Munoz N, Bosch F X, de Sanjose S, Herrero R, Castellsague X, Shah K V, Snijders P J, Meijer C J and International Agency for Research on Cancer Multicenter Cervical Cancer Study Group. Epidemiologic classification of human papillomavirus types associated with cervical cancer. New England Journal of Medicine. 2003; 348: 518-527; Koutsky L A et al., N Engl J Med, 2002, 347: 1645-51; http://www.bosa.co.kr/news_board/view.asp?news_pk=82896).

The HPV genome is about 8-10 kb in size and consists of a double-helical DNA enclosed in a capsid that resembles a golf ball. The genome structure of HPV can be roughly divided into early transcription region E (early gene region), late transcription region L (late gene region) and non-expression region LCR (long control region). The genome structure of HPV greatly affects the outbreak type, risk and prognosis of diseases. Particularly, E6 and E7 genes are integrated into the genome of an infected cell and play an important role in inducing cancer while they remain and are expressed there. The E6 and E7 genes of high-risk types of HPV such as HPV types 16 and 18 react with p53, E6AP, Rb (retinoblastoma, P105RB), P107, P130, etc., which are the most important tumor suppressor genes in human, and inactivate them. As a result, the infected cell is transformed into a cancer cell due to disorder of cell cycle regulation and apoptosis control mechanism. More than 99% of cervical cancer is caused by the high-risk type HPV and HPV gene fragments of E6/E7 are found almost always in the genome of the cancer cell. In contrast, since low-risk types of HPV have low ability to react with the tumor suppressor genes such as p53 or Rb and inactivate them, they normally do not cause cervical cancer. The largest gene of HPV is L1. L1 is present in most HPV types with the base sequence similarly conserved. HPV's capsid protein primarily consists of L1 and L1 has the highest antigenicity.

Once a cervical cell is malignantly transformed by HPV, it advances to so-called carcinoma in situ via precancerous lesion, dysplasia, CIN or squamous intraepithelial lesion (SIL). If the carcinoma in situ invades the basal layer under the cervical epithelium, it becomes carcinoma or invasive carcinoma. In 90% of women infected by HPV, the virus is naturally cleared from the body by the immune system. However, HPV remains in 10% of women who are infected with high-risk type HPV and induces precancerous lesions (Wallin K L, Winklund F, Angstrim T, et al: Type-specific persistence of human papillomavirus DNA before the development of invasive cancer. N Engl J Med 1999; 341: 1633; Bosch F X, Lorincz A, Munoz N, Meijer C J, Shah K V. The causal relation between human papillomavirus and cervical cancer. J Clin Pathol 2002; 55: 244-65). About 8% of the precancerous lesions advance to carcinoma in situ, and about 20% of carcinoma in situ develop into cancer. That is to say, infection of high-risk type HPV maintained 10-20 years or longer, it develops into cervical cancer and the frequency is estimated at about 0.16%. Since such a long time is necessary for the outbreak of cervical cancer and it occurs gradually, it is possible to treat or prevent cervical cancer by early diagnosing precancerous lesions. That is, cancer can be prevented by removing precancerous lesions of the cervix through conservative surgery.

HPV infection is hardly detected by culturing, staining, histological inspection or immunological inspection and can only be accurately diagnosed by genetic testing. There are three kinds of HPV genetic testing. The first is to simply investigate the presence of HPV. A representative example is amplification of the consensus sequence, i.e. invariant nucleotide sequence, of the HPV gene by PCR followed by identification through, for example, electrophoresis. The second is the so-called genotyping analysis of identifying not only the presence of HPV but also its type. The gold standard test is to perform PCR and analyze the genotype by automated nucleotide sequencing of the product. However, since this method requires a lot of cost, time and labor, it is being replaced by the HPV DNA microarray. A plurality of probes specific for HPV types are spotted on a solid support and a PCR product of the sample DNA is placed thereon and hybridized. Then, the result is analyzed using a scanner The third is intermediary of the two test methods. The hybrid capture assay (Digene Corporation, Gaithersburg, Md., USA) is an example. Although it allows to identify whether HPV exists and whether the HPV is high-risk type or low-risk type, accurate genotyping is impossible. In addition, only 13 high-risk type HPVs and 7 low-risk type HPVs can be identified, and other 20 or more HPV types cannot be identified (Kim K H, Yoon M S, Na Y J, Park C S, Oh M R, Moon W C. Development and evaluation of a highly sensitive human papillomavirus genotyping DNA chip. Gynecol Oncol. 2006; 100(1): 38-43; Selva L, Gonzalez-Bosquet E, Rodriguez-Plata M T, Esteva C, Sunol M and Munoz-Almagro C. Detection of human papillomavirus infection in women attending a colposcopy clinic. Diagnostic Microbiology and Infectious Disease. 2009; 64: 416-421).

Another important fact regarding HPV is that prevention of viral infection and cancer is possible through vaccination using the recently developed HPV vaccine. Two types of HPV vaccines are currently available. Gardasil (Merck & Co. Inc., Whitehouse Station, N.J., USA) is a quadrivalent vaccine prepared against HPV types 16, 18, 6 and 11. The other, Cervarix (GlaxoSmithKline Biologicals, Rixensart, Belgium), is a bivalent vaccine designed to prevent infection from HPV types 16 and 18. These vaccines are the most effective for adolescent girls before sexual activity, and the efficacy decreases in women who have been infected by HPV16 or HPV18 before. For this reason, vaccination to adult women is controversial, but, it may be possible unless the HPV infection is by type 16 or 18. Accordingly, it is becoming more and more important to identify not just the HPV infection but the accurate type of HPV (Selva L, Gonzalez-Bosquet E, Rodriguez-Plata M T, Esteva C, Sunol M and Munoz-Almagro C. Detection of human papillomavirus infection in women attending a colposcopy clinic. Diagnostic Microbiology and Infectious Disease. 2009; 64: 416-421; Reynales-Shigematsu L M, Rodrigues E R, Lazcano-Ponce E. Cost-effectiveness analysis of a quadrivalent human papilloma virus vaccine in Mexico. Arch Med Res. 2009 August; 40(6): 503-13).

The Papanicolaou test (Papanicolaou smear or Pap smear) of examining cervical cells has been used as a primary screening test of cervical cancer. However, since the Pap smear is a subjective test, false positive results are not infrequent and, thus, a test method for complementing it has been necessary. Actually, the cytological test based on Pap smear is not so effective for diagnosis of HPV infection, which is the most important cause of cervical cancer, and it is not easy to predict whether an abnormal lesion will be disappear naturally or progress to cancer. Indeed, it is impossible to diagnose non-symptomatic or latent infection through cytomorphological examination under a microscope, particularly to distinguish infection by high-risk type HPV from that by low-risk type HPV. Accordingly, to reduce cervical cancer, a diagnosis method capable of monitoring HPV infection, risk thereof and genotype thereof is required.

As described above, it is necessary to test the presence of HPV and its genotype accurately and quickly, at low cost and in large scale. The DNA microarray (chip) technique is the most suitable in this sense.

HPV diagnosis products used overseas include Hybrid Capture II (Qiagen, Germany; approved by the FDA), Cervista™ HPV HR test (Hologic Women's Health Co.; 14 high-risk types; approved by the FDA), Roche AMPLICOR HPV test (Roche Molecular Systems, USA; CE marking), PapilloCheck HPV screening test kit (Greiner Bio-One GmbH, Germany; 18 high-risk types and 6 low-risk types; CE marking) and Digene HPV genotyping RH test (Qiagen; high-risk types; CE marking).

However, the currently commercialized HPV genotyping DNA chips have the following disadvantages.

Firstly, the number of HPV genotypes that can be tested is limited.

Secondly, although the HPV probes need to be designed based on the base sequence information of the HPV genome of actual clinical samples, most of the HPV DNA chips are designed based on the standard base sequence available from literatures or US GenBank. Since there are numerous variations in the DNA base sequence of the HPV genome, if primers or probes are designed without considering them, PCR or hybridization may not be carried out as desired and error may occur.

Thirdly, since an internal reference gene (control gene) is not used, it is not easy to known whether a negative result is true negative or false negative.

Fourthly, the so-called universal probe capable of testing the presence of all genotypes of HPV is not considered. For this reason, when a negative result is obtained for all the HPV genotypes, it is not easy to determine whether it means that no HPV exists in the sample or other genotypes of HPV may exist.

Fifthly, PCR is the most important step prior to HPV DNA analysis, but the condition is not standardized.

Sixthly, for standardization of the HPV DNA chip and HPV genotyping using same, standard materials for gene cloning are required for each genotype of HPV.

Seventhly, although many HPV DNA diagnosis kits are available, large-scale testing and comparison for investigating how accurate they are as compared to the standard test and how useful they are for screening of cervical cancer and precancerous lesions are insufficient.

The inventors of the present disclosure have studied the presence of anogenital HPVs, types thereof and DNA base sequences thereof for more than 250,000 samples for several years through post-PCR sequencing, DNA microarray testing, and HPV type-specific PCR, and so forth. Based on the result and analysis of the features of commercially available HPV DNA diagnosis kits, they have noticed the problems of the existing art to be solved and invented a new HPV DNA microarray. Details are as follows.

1. Type and Number of Genital and Anal HPVs

According to the literatures, the number of HPV types that can invade the genital and anal regions including the cervix are estimated at about 40 but is not clear. For accurate diagnosis of all the types of genital HPVs, it is prerequisite to test multiple samples for all the types of genital HPVs. However, such data are rare worldwide.

Thus, the inventors of the present disclosure have performed PCR for L1, L2 and E6/E7 genes of HPV for about 16,000 cervical samples from Korean women and analyzed the base sequence of all the PCR products. Based on these data, and referring to the reports from the US and other countries, they have determined the HPV types that should be included in the new HPV DNA chip. The number of the types was 43 and, thus, they have invented a DNA chip capable of analyzing all the 43 types of genital HPVs. This will be described in detail in Example 1.

2. Standard Materials

One of the basic requirements in HPV genotyping is that all standard materials (reference materials) should be prepared for each genotype. This may be HPV itself, the entire genome of HPV, the key genes of HPV or plasmid clones. The kind and number of the standard materials of genital HPVs disclosed in literatures and deposited in GenBank are very restricted.

As described earlier, the inventors have performed PCR for the L1, L2 and E6/E7 genes of HPV for about 15,000 cervical samples from Korean women and analyzed the base sequence of all the PCR products. Based on the result, they have obtained plasmid DNA clones by cloning the L1, L2 and E6/E7 genes for 43 types of genital HPV wholly or partially. They have decided to identify the genotype of the 43 types of HPV by targeting specific regions of the HPV L1 gene and determined plasmid standard materials of HPV L1 gene clones for each type. They were used for the development of a DNA chip and quality control (QC) thereof. This will be described in detail in Example 2.

3. PCR Amplification

For accurate and sensitive analysis is possible using the HPV DNA chip, PCR amplification needs to be performed adequately first. For this, the PCR condition for amplifying the HPV L1 gene to be hybridized on the HPV DNA chip of the present disclosure should be optimized and, most of all, the PCR primers should be designed adequately. Further, it is preferred that the amplification of HPV L1 gene and reference and control genes is achieved in a single tube under the same condition by a single duplex PCR. Since the HPV PCR condition reported in literatures or recommended for the commercially available HPV DNA chips is frequently nested PCR, the amplification process is inconvenient and the risk of contamination is high. Further, some types of HPV are amplified well but others are not and interference often occurs when the reference gene is amplified together.

Thus, through repeated experiments, the inventors have newly established the base sequence of oligonucleotide primers for PCR and the amplification condition based on the base sequence of L1 gene of the 43 types of HPV and standard materials as described earlier. As a result, the amplification of the HPV L1 gene and reference gene could be achieved by a single duplex PCR. This will be described in detail in Example 3.

4. Control Gene

One of the basic requirements in HPV DNA chip analysis is that not only the target gene but also the internal reference or control gene therefor should be investigated as well. This is essential for normalization analysis of the signals from the DNA chip and for distinction from false negative and false positive results. Nonetheless, a number of DNA chip tests are carried out without control genes.

The inventors of the present disclosure have used the human beta-globin gene as a control gene. Further, they have found out that the housekeeping gene beta-actin may be used as another control gene and newly added it in the HPV DNA chip. This will be described in detail in Examples 4-6.

5. Probe Structure

The most important thing in HPV genotyping DNA microarray testing is that hybridization is performed adequately for each genotype of HPV so that it can be identified accurately. The probe is of great importance in this aspect. As described above, the inventors of the present disclosure have performed PCR for L1 gene of HPV for more than 15,000 cervical samples from Korean women and analyzed the base sequence of all the PCR products. Based on the result, they have established plasmid DNA clone standard materials for 43 types of genital HPVs and have determined the basic oligonucleotide structure of the HPV DNA chip. The oligonucleotide is from 18 to 30 base pairs (bp) long. This will be described in detail in Example 5.

6. Final Design and Production of Probe

In general, an oligonucleotide probe is 20-30 by long and has a C6 linker attached thereto. However, the inventors of the present disclosure have empirically found out that a problem may occur during spotting on a glass slide in that case owing to spatial instability.

Thus, the inventors of the present disclosure have designed an oligonucleotide probe having a longer C20 linker This will be described in detail in Example 5. In addition, they have designed a d-shaped probe by introducing a stem part. This will be described in detail in Example 6.

7. Fabrication of DNA Microarray (Chip)

A grid was designed according to the probe and the probe mixed in an adequate buffer was spotted on a glass slide for a microscope. This will be described in detail in Example 7.

8. Reaction on DNA Microarray (Chip)

100 artificial standard samples obtained from various combinations of one, two or three clones for each type of HPV were used as templates for PCR amplification of HPV L1 and beta-actin genes. The PCR products were placed on the chip and hybridization was performed at least 3 times. Then, the optimal condition was established by analyzing with a fluorescence scanner. This will be described in detail in Example 8.

9. Evaluation of Accuracy of DNA Microarray (Chip)

The fabricated new HPV DNA chip of the present disclosure was compared with that of the standard sequencing and HPV-type specific PCR to investigate the accuracy, sensitivity and specificity. Further, it was investigated whether the HPV DNA chip can be used to test the presence of HPV in a clinical sample such as a cervical cell and the genotype thereof. This will be described in detail in Example 9. The existing HPV DNA chips lack such data.

10. Evaluation of Accuracy of Early Diagnosis of Cervical Cancer

The accuracy, sensitivity and specificity of diagnosis of cervical cancer and precancerous lesions of the novel HPV DNA chip fabricated according to the present disclosure were compared with those of the existing Hybrid Capture Assay 2 (HCA-2). In addition, it was investigated whether the HPV DNA chip of the present disclosure can be used to predict cervical cancer or precancerous lesions from a clinical sample such as a cervical cell. This will be described in detail in Example 10. The existing HPV DNA chips lack such data. The HPV DNA chip of the present disclosure was confirmed to be clinically applicable.

The present disclosure is directed to providing a DNA chip for diagnosing HPV capable of accurately and quickly diagnosing infection by 44 types of genital HPV simultaneously.

The present disclosure is also directed to providing an oligonucleotide probe and a PCR primer capable of accurately detecting 44 types of genital HPV with high specificity and sensitivity.

The present disclosure is also directed to providing a kit for genotyping 44 types of genital HPV in which the HPV DNA chip, the PCR primer, a label, etc. are provided “all in one”.

In one general aspect, the present disclosure provides a DNA chip for genotyping human papillomavirus (HPV) from a sample, including a linear oligonucleotide probe having a base sequence selected from SEQ ID NOS 6-109.

In another general aspect, the present disclosure provides a DNA chip for genotyping HPV from a sample, including a d-shaped oligonucleotide probe having a base sequence selected from SEQ ID NOS 110-213.

The DNA chip of the present disclosure is capable of simultaneously genotyping 44 types of HPV including: HPV-16, HPV-18, HPV-31, HPV-33, HPV-35, HPV-39, HPV-45, HPV-51, HPV-52, HPV-56, HPV-58, HPV-59, HPV-68a, HPV-68b and HPV-82 as high-risk type HPVs; HPV-26, HPV-53, HPV-66, HPV-67, HPV-69, HPV-70 and HPV-73 as moderate-risk type HPVs; HPV-6, HPV-11, HPV-34, HPV-40, HPV-42, HPV-43, HPV-44, HPV-54, HPV-55, HPV-61, HPV-62, HPV-72 and HPV-81 as low-risk type HPVs; and HPV-90, HPV-10, HPV-27, HPV-30, HPV-32, HPV-57, HPV-83, HPV-84 and HPV-91 as other HPVs.

In an exemplary embodiment of the present disclosure, the oligonucleotide probe having a base sequence selected from SEQ ID NOS 6-97 or SEQ ID NOS 110-201 may bind complementarily to L1 gene region specific for each type of HPV.

In an exemplary embodiment of the present disclosure, the oligonucleotide probe having a base sequence selected from SEQ ID NOS 98-105 or SEQ ID NOS 202-209 may be a universal probe binding complementarily to L1 gene region existing in all types of HPV.

In an exemplary embodiment of the present disclosure, the oligonucleotide probe having a base sequence selected from SEQ ID NOS 106-109 or SEQ ID NOS 210-213 may bind complementarily to beta-actin gene as positive control.

In an exemplary embodiment of the present disclosure, the DNA chip may have 8-24 partitioned wells on which the probe can be spotted.

In an exemplary embodiment of the present disclosure, the concentration of the oligonucleotide probe may be at least 38 pmol.

In an exemplary embodiment of the present disclosure, C6 amine-modified dideoxythymidine may be attached to the oligonucleotide probe as a linker so as to spot the oligonucleotide probe on a superaldehyde-coated support.

In an exemplary embodiment of the present disclosure, the support may be selected from a group consisting of glass slide, paper, nitrocellulose membrane, microplate well, plastic, silicon, DVD and bead.

In an exemplary embodiment of the present disclosure, the sample may be selected from a group consisting of cervical swab, vaginal swab, cervical tissue, penile tissue, urine, anal tissue, rectal tissue, pharyngeal tissue, oral tissue and head and neck tissue.

In an exemplary embodiment of the present disclosure, the sample may be selected from a group consisting of penile cancer cell, urethral cancer cell, anal cancer cell, head and neck cancer cell and precancerous cells thereof

In an exemplary embodiment of the present disclosure, the DNA chip may be used to determine whether HPV vaccine will be administered.

In another general aspect, the present disclosure provides a kit for genotyping HPV, including the DNA chip, a primer for amplifying a target gene by PCR and a label for detecting the amplified DNA.

In an exemplary embodiment of the present disclosure, the primer may be a primer for amplifying human beta-actin gene having a base sequence selected from SEQ ID NOS 1-2 or a primer for amplifying HPV L1 gene having a base sequence selected from SEQ ID NOS 3-5.

In an exemplary embodiment of the present disclosure, the label the may be one or more selected from a group consisting of Cy3, Cy5, Cy5.5, BODIPY, Alexa 488, Alexa 532, Alexa 546, Alexa 568, Alexa 594, Alexa 660, rhodamine, TAMRA, FAM, FITC, Fluor X, ROX, Texas Red, Orange Green 488X, Orange Green 514X, HEX, TET, JOE, Oyster 556, Oyster 645, BODIPY 630/650, BODIPY 650/665, Calfluor Orange 546, Calfluor Red 610, Quasar 670, biotin, Au, Ag and polystyrene.

In another general aspect, the present disclosure provides a method for genotyping HPV, including:

(a) amplifying a target gene of a sample by single, duplex or nested PCR using a primer having a base sequence selected from SEQ ID NOS 1-5;

(b) labeling an oligonucleotide probe of a DNA chip;

(c) hybridizing the labeled probe with the amplified PCR product; and

(d) detecting the hybridized product.

In an exemplary embodiment of the present disclosure, the labeling in (b) may be performed by labeling the oligonucleotide probe with a label selected from a group consisting of Cy3, Cy5, Cy5.5, BODIPY, Alexa 488, Alexa 532, Alexa 546, Alexa 568, Alexa 594, Alexa 660, rhodamine, TAMRA, FAM, FITC, Fluor X, ROX, Texas Red, Orange Green 488X, Orange Green 514X, HEX, TET, JOE, Oyster 556, Oyster 645, BODIPY 630/650, BODIPY 650/665, Calfluor Orange 546, Calfluor Red 610, Quasar 670 and biotin.

In an exemplary embodiment of the present disclosure, the labeling in (b) may be performed by labeling a target probe first with an Au nanoparticle and then with silver staining and binding the target probe complementarily to the oligonucleotide probe of the DNA chip.

In an exemplary embodiment of the present disclosure, the labeling in (b) may be performed by labeling a target probe first with an Au nanoparticle and then forming a silver shell and binding the target probe complementarily to the oligonucleotide probe of the DNA chip.

In an exemplary embodiment of the present disclosure, the target probe may have a base sequence selected from SEQ ID NOS 214-215 and C18 linker, A10 and thiol group may be sequentially attached at the 3′-terminal.

In an exemplary embodiment of the present disclosure, the genotyping method may further include analyzing using plasmid vectors in which L1 genes of the 65 types of HPV described in Table 1 are inserted as positive control clones.

In an exemplary embodiment of the present disclosure, the sample may be selected from a group consisting of cervical swab, vaginal swab, cervical tissue, penile tissue, urine, anal tissue, rectal tissue, pharyngeal tissue, oral tissue and head and neck tissue.

In an exemplary embodiment of the present disclosure, the sample may be selected from a group consisting of penile cancer cell, urethral cancer cell, anal cancer cell, head and neck cancer cell and precancerous cells thereof.

The oligonucleotide probe for genotyping HPV, the DNA chip and the diagnosis kit including same and the method for genotyping HPV according to the present disclosure were completed in nine steps as follows.

1. Preparation of Standard and Control Samples

The inventors of the present disclosure performed PCR for L1, L2 and E61E7 genes of HPV for about 16,000 cervical samples from Korean women and analyzed the base sequence of all the PCR products. Based on these data, and referring to the reports from the US and other countries, they determined the HPV types that should be included in a new HPV DNA chip. The number of the types was 43 and, thus, they invented a DNA chip capable of analyzing all the 43 types of genital HPVs.

2. Isolation of DNA

DNA was isolated from the samples obtained in the step 1 using an adequately established method.

3. Duplex PCR

Oligonucleotide primers for amplifying HPV L1 gene and human beta-actin gene were designed and adequate PCR condition was established. PCR was performed in duplex and condition was established for each gene for different primer concentrations. PCR was performed for HPV L1 gene and human beta-actin gene using the DNA isolated in the step 2 as template.

4. Sequencing and Cloning

After the PCR, base sequence of the HPV L1 gene was analyzed by sequencing and a database was made based on the result. The PCR product whose HPV type was identified was cloned into a plasmid vector. Later, the clones were used as standard and control samples during the establishment of reaction condition for the DNA chip of the present disclosure. The clinical DNA samples whose HPV genotype was identified were stored and used for accuracy analysis of the DNA chip of the present disclosure.

5. Probe Design

Based on the sequence database built in the step 4 by genotyping HPV from cervical cells and cancer tissues of Koreans and foreign HPV-related databases, an oligonucleotide probe complementary to L1 gene of all the 43 types of HPV that can infect human cervix and human beta-actin gene and capable of detecting them through hybridization on the DNA chip was designed. Also, a d-shaped oligonucleotide probe having a stem part was designed.

6. Fabrication of DNA Chip

A grid on which the probe designed in the step 5 will be spotted was designed and the probe mixed with an adequate buffer was spotted (or arrayed) on a glass slide for a microscope. The resulting DNA chip was subjected to stabilization and quality control.

7. Establishment of Reaction and Analysis Condition on DNA Chip

HPV L1 and beta-actin genes were amplified by duplex PCR using various combinations of one, two or three clones for each type of HPV obtained in the step 4 as templates. The PCR products were placed on the DNA chip and hybridization was performed several times. Then, the optimal condition was established by analyzing with a fluorescence scanner.

8. Analysis of Clinical Sample on DNA Chip

The DNA of the clinical samples of which the presence and type of HPV were identified in the steps 3 and 4 by PCR and sequencing was subjected again to duplex PCR. The PCR product was placed on the DNA chip fabricated in the step 6 and subjected to hybridization under the condition established in the step 7. After washing, the result was analyzed using a fluorescence scanner. Through this, sensitivity, specificity and reproducibility of the DNA chip of the present disclosure were analyzed and the optimal condition for diagnosis of HPV genotype using the DNA chip of the present disclosure was established again.

9 Analysis of Correlation with Clinical Data Following Analysis of Clinical Sample on DNA Chip

The result of post-PCR DNA chip analysis in the step 8 was compared with clinical data such as those of Pap smear and their correlation was investigated. It was analyzed whether the DNA chip of the present disclosure is useful in predicting cervical cancer or precancerous lesions. As a result, it was confirmed that the DNA chip of the present disclosure is useful not only in genotyping of HPV but also in screening of cervical cancer.

A diagnosis kit using the DNA chip of the present disclosure provides 1) a reagent for extracting DNA from a sample such as cervical swab, paraffin section, etc., 2) a reagent for amplifying HPV L1 and beta-actin genes by PCR, 3) a plasmid DNA clone used as positive control during the amplification of HPV gene, 4) the oligo DNA chip for genotyping HPV and 5) a reaction solution for hybridization using the DNA chip and a washing solution “all in one”.

In accordance with the present disclosure, all the 44 types of HPV invading the genitalia can be detected and coinfection by more than one type of HPV can be diagnosed accurately. The sensitivity and specificity of HPV genotyping is close to 100% and a number of samples can be tested quickly. The present disclosure is very useful in predicting cervical cancer and precancerous lesions.

In particular, the DNA chip for genotyping HPV according to the present disclosure and the kit using same are very useful in large-scale automated diagnosis of infection of samples such as cervical swab, vaginal swab, urine, anal tissue, oral tissue, pharyngeal tissue, etc. by HPV and genotyping thereof. Also, they may be used together with Pap smear or alone to screen cervical cancer and precancerous lesions thereof, reducing cost, labor and time of test. Also, they are useful for customized vaccination since the genotype of HPV can be analyzed accurately.

Accordingly, the present disclosure will contribute greatly to the improvement of health and well-being by reducing HPV-related cancers and deaths caused thereby and is very valuable in medical industry.

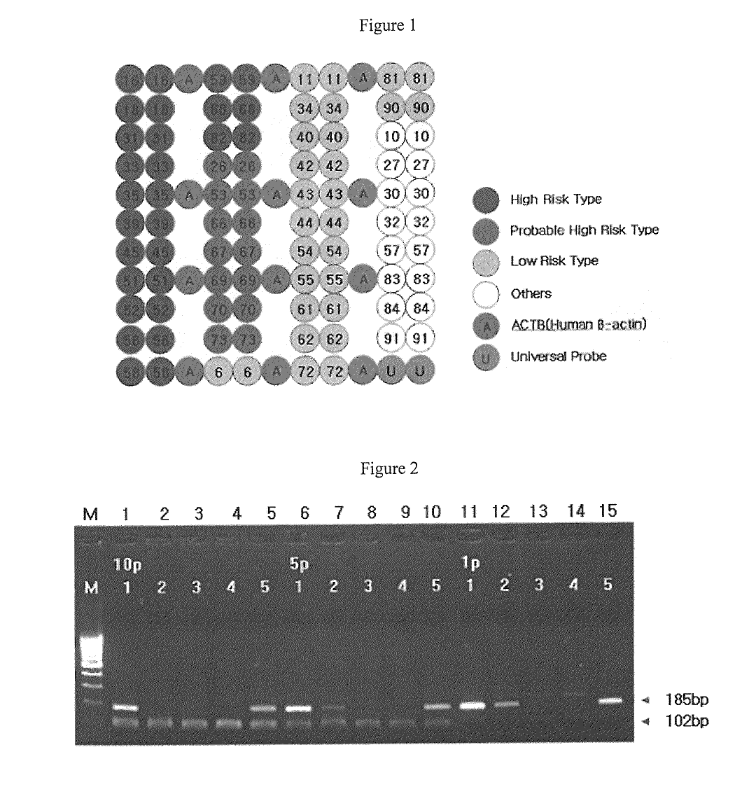

FIG. 1 shows a grid of a DNA microarray (chip) for genotyping HPV according to the present disclosure. Eight wells were formed on one DNA chip and a probe specific for HPV L1 gene of each type, a universal probe common to all types of HPV L1 gene and a probe for a control or reference gene was spotted on each well. In FIG. 1, the red spots correspond to cancer-causing 14 high-risk type HPVs: HPV 16, 18, 31, 33, 35, 39, 45, 51, 52, 56, 58, 59, 68 and 82. The pink spots correspond to 7 probably high-risk type HPVs that are likely to cause cancer although not clearly validated: HPV 26, 53, 66, 67, 69, 70 and 73. The sky blue spots correspond to 14 low-risky type HPVs: HPV-6, 11, 34, 40, 42, 43, 44, 54, 55, 61, 62, 72, 81 and 90. The yellow spots correspond to 8 other HPV types whose risk of cancer is not elucidated yet: HPV 10, 27, 30, 32, 57, 83, 84 and 91. The purple spots, corresponding to universal probes, give positive results when HPV is present in the sample, regardless of type. The green spots correspond to control gene probes serving as corner marker and indicating that DNA was successfully extracted from the sample. In the present disclosure, human beta-actin (ACTB) gene, which is one of the so-called housekeeping genes, was used as control gene.

FIG. 2 is an electrophoresis image showing an experimental result for determining optimal concentration ratio of HPV L1 primers and. ACTB primers for amplifying HPV L1 gene, which is a target gene, and human beta-actin gene, which is a control gene, by duplex PCR. My11, GP6-1 and GP6+ were used as HPV L1 primers and ACTBF and ACTBR were used as beta-actin primers. Lane M: 100 by size marker; lanes 1-5: 10 pmol HPV L1 primer, 10 pmol ACTB primer; lanes 6-10: 10 pmol HPV L1 primer, 5 pmol ACTB primer; lanes 11-15: 10 pmol HPV L1 primer, 1 pmol ACTB primer. Sample 1: human cervical swab sample positive for HPV type 56; sample 2: human cervical swab sample positive for HPV type 16; samples 3-4: cervical swab samples not infected by HPV; sample 5: HeLa cervical cancer cell sample including the gene of HPV type 18 as positive standard material. The conditions of lanes 6-10 were confirmed as the best conditions for duplex PCR.

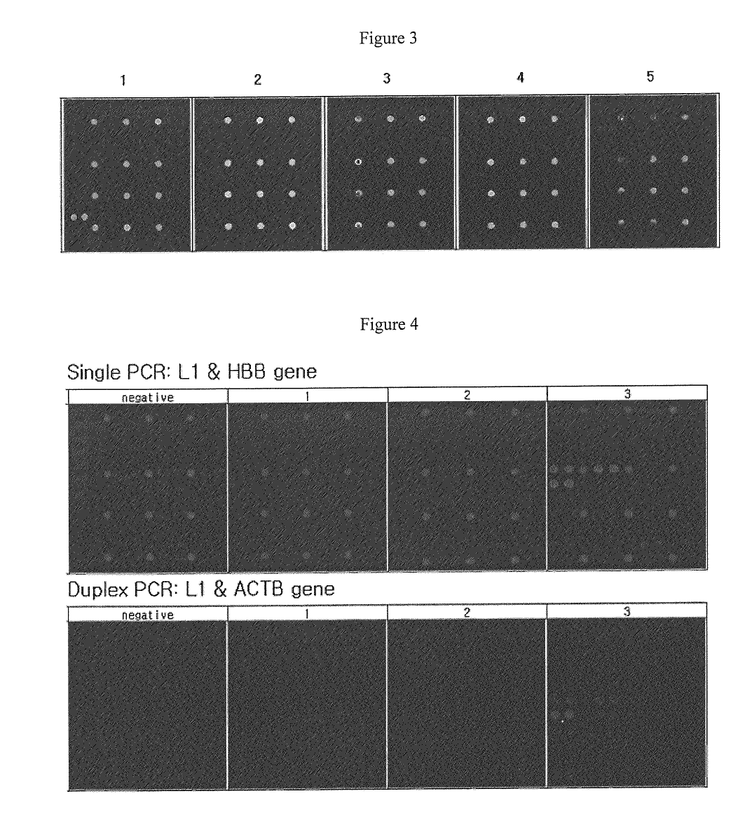

FIG. 3 shows a result of performing hybridization after placing the samples of the lanes 6-10 in FIG. 2 on the HPV DNA chip of the present disclosure and scanning with a fluorescence scanner at a wavelength of 635 nm

FIG. 4 shows an experimental result of performing single PCR of HPV L1 gene and beta-globin gene separately according to the existing method and performing duplex PCR with a sample that exhibited negative result for HPV and non-specific low sign. Samples 1-2 are gDNA samples of HEK cell as HPV-uninfected negative control and sample 3 is a cervical swab sample coinfected by HPV 35, HPV 39, HPV-53, HPV 58, HPV 72 and HPV-66.

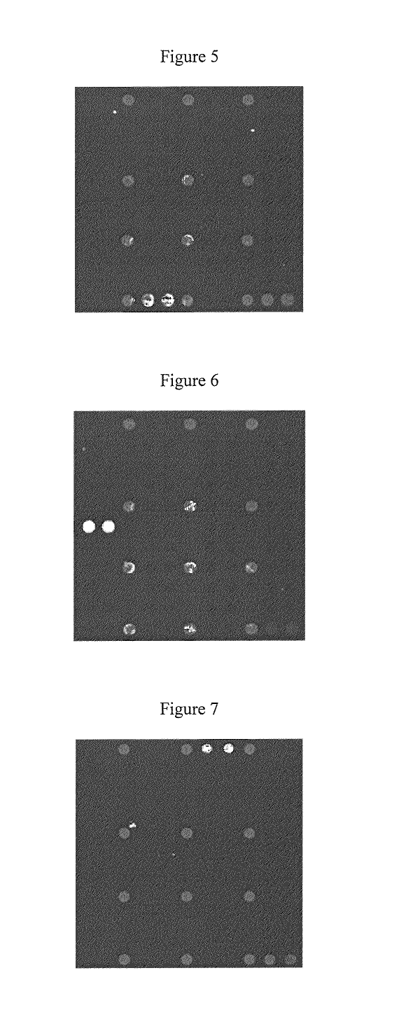

FIG. 5 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV-6 L1 gene, a universal probe and a beta-actin probe and was diagnosed to be infected by HPV type 6. Since the sample gave positive results for the universal probe and the beta-actin probe, it was determined as true positive, not false positive. This result was also confirmed through sequencing.

FIG. 6 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV 39 L1 gene, a universal probe and a beta-actin probe and was diagnosed to be infected by HPV type 39. This result was also confirmed through sequencing.

FIG. 7 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV 11 L1 gene, a universal probe and a beta-actin probe and was diagnosed to be infected by HPV type 11. This result was also confirmed through sequencing.

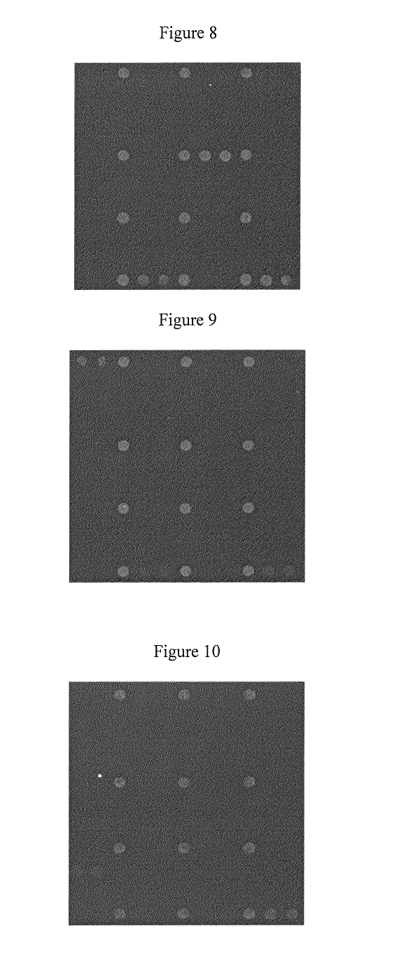

FIG. 8 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV-6 L1 gene, a probe specific for HPV 43 L1 gene, a universal probe and a beta-actin probe and was diagnosed to be coinfected by HPV-6 and HPV-43 (mixed infection). This result was also confirmed through sequencing.

FIG. 9 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV-6 L1 gene, a probe specific for HPV 11 L1 gene, a universal probe and a beta-actin probe and was diagnosed to be coinfected by HPV-6 and HPV-11. This result was also confirmed through sequencing.

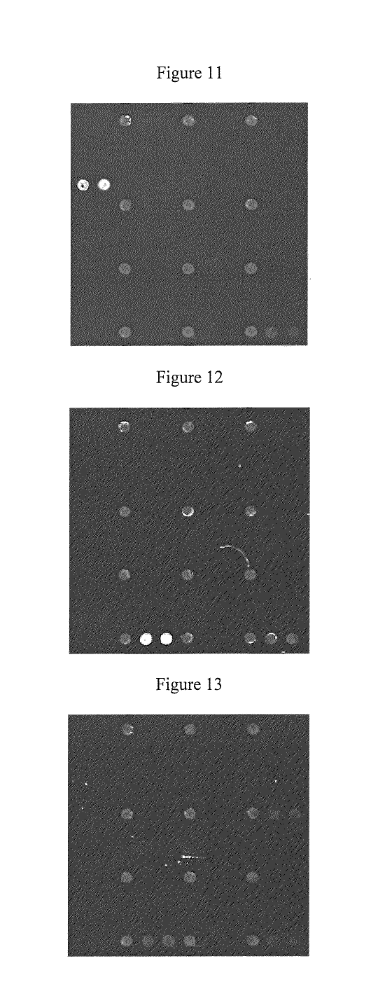

FIG. 10 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV 52 L1 gene, a universal probe and a beta-actin probe and was diagnosed to be infected by HPV type 52. This result was also confirmed through sequencing.

FIG. 11 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV 33 L1 gene, a universal probe and a beta-actin probe and was diagnosed to be infected by HPV type 33. This result was also confirmed through sequencing.

FIG. 12 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV-6 L1 gene, a probe specific for HPV 56 L1 gene, a universal probe and a beta-actin probe and was diagnosed to be coinfected by HPV-6 and HPV-56. This result was also confirmed through sequencing.

FIG. 13 shows an exemplary result of extracting DNA from cervical and vaginal swab samples of a Korean woman, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV-6 L1 gene, a probe specific for HPV 30 L1 gene, a universal probe and a beta-actin probe and was diagnosed to be coinfected by HPV-6 and HPV-30. This result was also confirmed through sequencing.

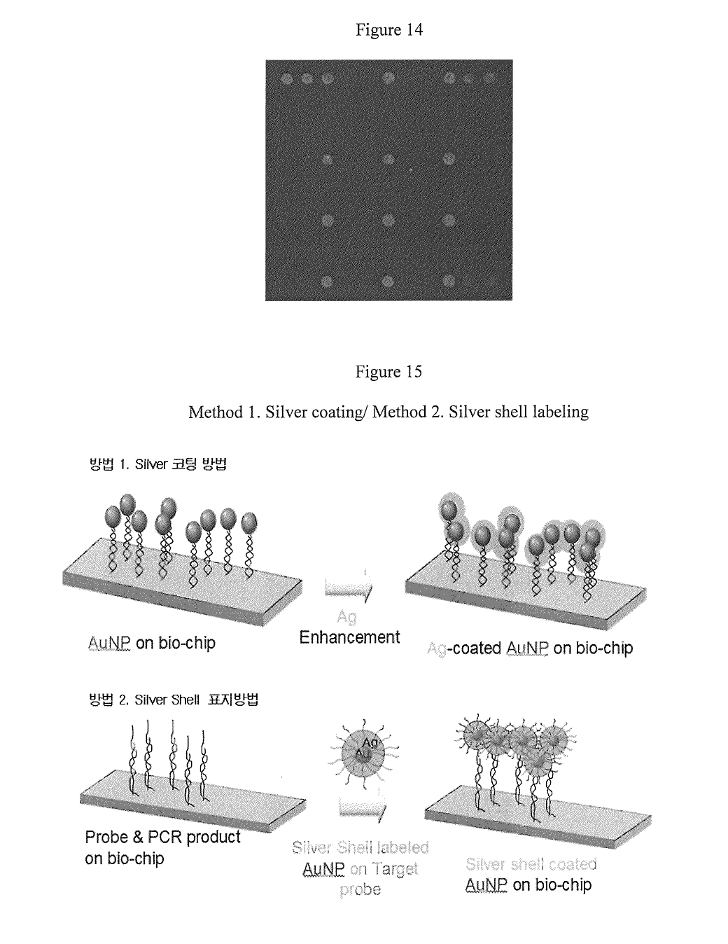

FIG. 14 shows an exemplary result of extracting DNA from a cervical swab sample of a Korean woman who had high grade squamous intraepithelial lesion histologically identified in the cervix, performing duplex PCR according to the present disclosure, performing hybridization of the HPV L1 and beta-actin amplification products on the HPV DNA chip of the present disclosure and scanning with a fluorescence scanner after washing. The sample exhibited positive results for a probe specific for HPV 16 L1 gene, a probe specific for HPV 81 L1 gene, a universal probe and a beta-actin probe and was diagnosed to be coinfected by HPV-16 and HPV-81. This result was also confirmed through sequencing.

FIG. 15 schematically shows a process of labeling, after probes spotted on a chip are hybridized with PCR products, first with gold nanoparticles (AuNP) and then with silver.

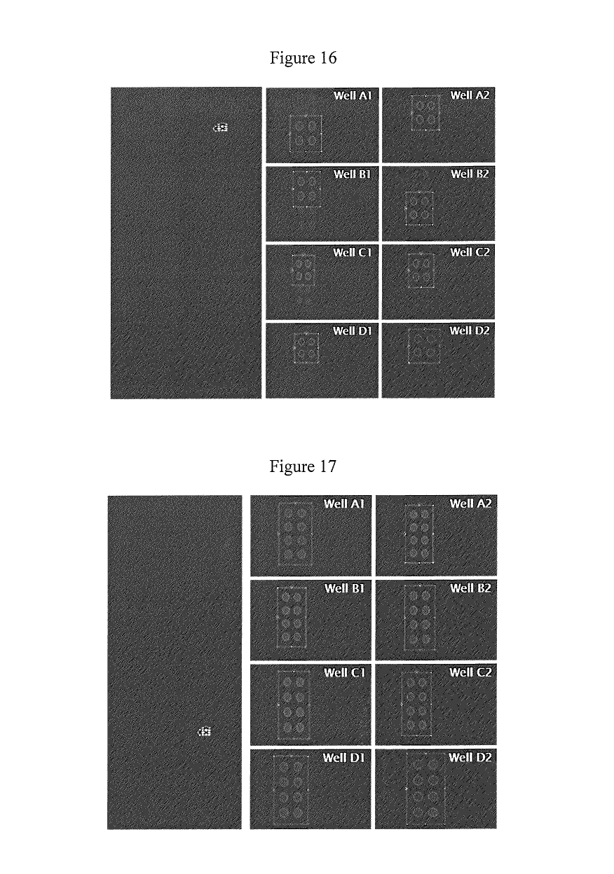

FIG. 16 shows scanning images of an HPV-6-AuNP-Ag enhancement chip. The images on the left side show a result of scanning all the 8 wells, and the images on the right side show spots spotted in each well.

FIG. 17 shows scanning images of an HPV-6-AuNP-Ag core shell chip. The images on the left side show a result of scanning all the 8 wells, and the images on the right side show spots spotted in each well. Unlike the silver (Ag) staining images of FIG. 16, the spots are clearly shown.

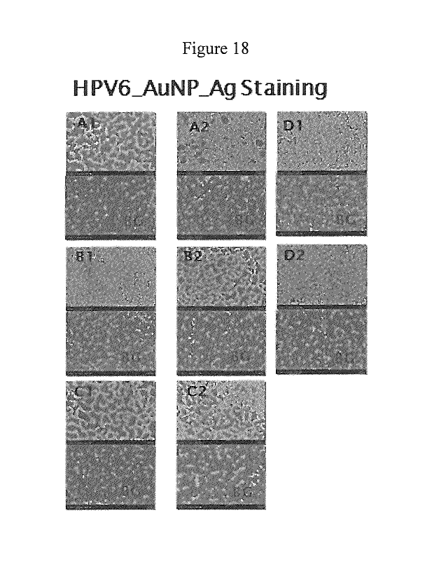

FIG. 18 shows a result of analyzing the spots and background (BG) of HPV-6-AuNP-Ag stained chip by scanning electron microscopy (SEM). It was confirmed that gold nanoparticles were present with high density in each spot.

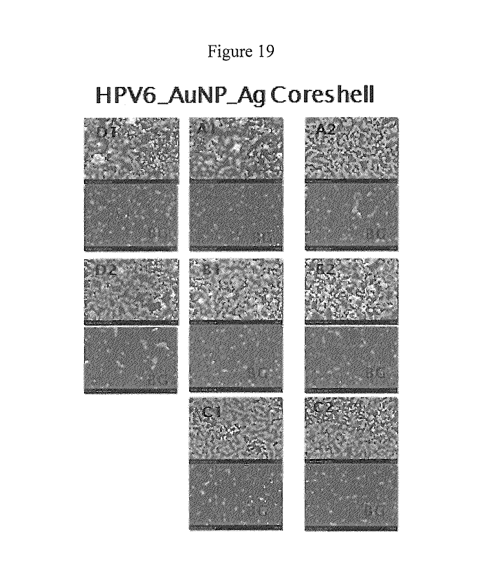

FIG. 19 shows a result of analyzing the spots and background (BG) of HPV-6-AuNP-Ag core shell chip by SEM. It was confirmed that gold nanoparticles were present with high density in each spot.

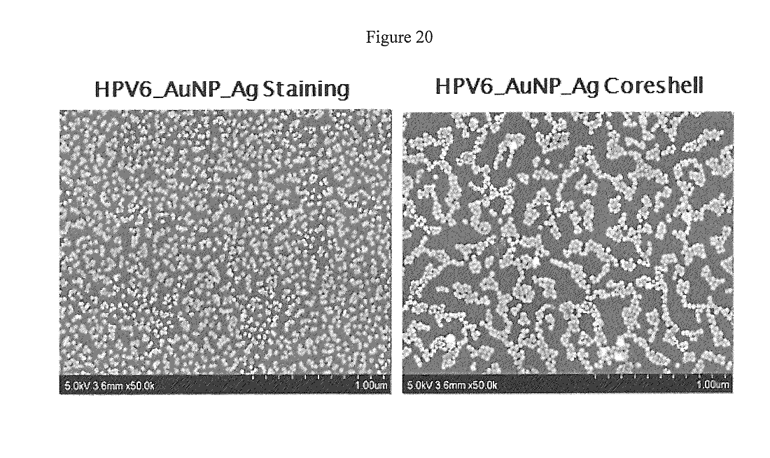

FIG. 20 shows SEM images of HPV-6-AuNP-Ag enhanced spots and HPV-6-AuNP-Ag core shell-labeled spots. It can be seen that Ag core shell labeling gives much more stable result than Ag staining. In case of Ag staining, the staining was non-specific.

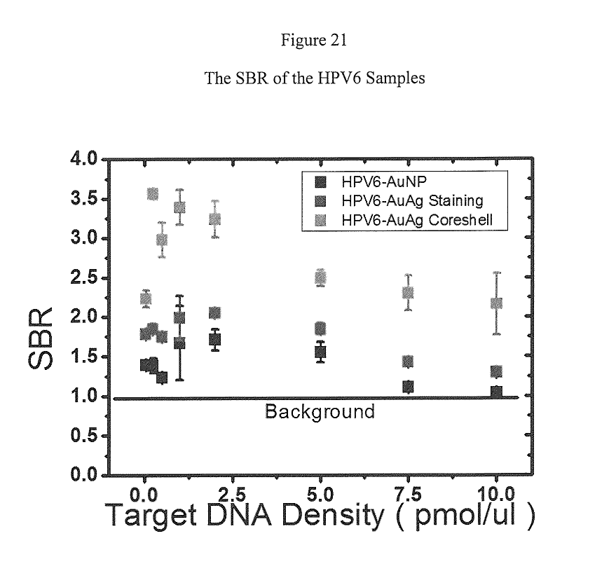

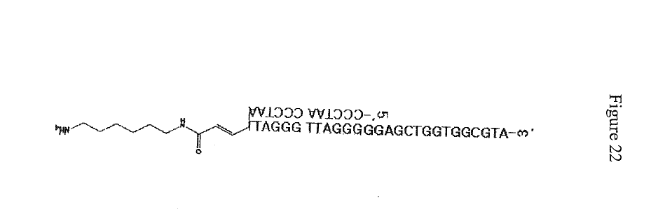

FIG. 21 shows a result of scanning a chip wherein a PCR template for HPV-6 and a target probe (LTP) are labeled with AuNP (HPV-6-AuNP), a chip wherein a PCR template for HPV-6 and a target probe (LTP) are labeled with AuNP and then enhanced with silver (HPV-6-AuAg staining) and a chip wherein a PCR template for HPV-6 and a target probe (LTP) are labeled first with Au and then with Ag core shell (HPV-6-AuAg Core shell) at different template concentrations using a scanner equipped with a PD and comparing reflectivity of each spot with SBR.

FIG. 22 schematically shows an exemplary structure of a d-shaped probe used in a DNA chip.

Hereinafter, the present disclosure will be described in more detail through examples. But, the present disclosure is not limited by the following examples.

### EXAMPLE 1

### Preparation of Control Sample and Extraction of DNA

Samples to be used as standard materials were prepared and DNA was extracted therefrom.

As a first sample, human cervical cancer cell of which infection by HPV and type thereof are identified and which have been widely used in HPV genotyping studies was purchased from ATCC (Manassas, Va.20108, USA) and Korea Cell Line Bank (KCLB; Seoul National University Cancer Research Institute, Korea) and used after monolayer culturing. Genomic DNA was isolated therefrom.

A second sample was obtained from the CIN cervical tissue of 100 Korean women who were diagnosed as cervical cancer or carcinoma in situ. Formalin-fixed and paraffin-embedded tissues were cut into 5-10 sections of 10-μm thickness, and attached to a glass slide for a microscope. Then, only the cancer cells were microdissected. Among the 100 cervical cancer lesions, 98 were cervical intraepithelial neoplasm (CIN).

As a third sample, cervical samples were obtained from 15,708 women who visited Hamchun Diagnosis Center (Seoul, Korea) or Korea Gynecologic Cancer Foundation (Seoul, Korea) from 2005 to 2007 and received cervical swab and Pap smear test. Their age was between 16 and 80 years and the average age was 47 years.

DNA was isolated from the samples as follows.

To extract DNA from the cells, cervical swab samples and paraffin section samples, DNA was concentrated and purified using the Labo Pass™ tissue mini kit (CME0112, Cosmo Genetech, Korea). Details are as follows.

A. Isolation of Genomic DNA from Cells

Monolayer cultured cells were isolated and introduced into a 50-mL centrifuge tube. After centrifugation at 3500 rpm for 30 minutes, the supernatant was discarded and pellets were resuspended in 500 μL of PBS and transferred to a 1.5-mL centrifuge tube. After centrifugation again at 12,000 rpm for 2 minutes, the remaining medium was removed by washing and genomic DNA was obtained.

B. Isolation of Genomic DNA from Cervical Swab Sample

1) 1.5 mL of sample solution is transferred to a 1.5-mL centrifuge tube. Cells are settled by centrifuging at 13,500×g for 2 minutes.

2) The supernatant is removed and 500 μL of PBS is added.

3) The cells are mixed well with the solution using a vortex.

4) After centrifugation at 13,500×g for 2 minutes, the supernatant is removed.

5) 200 μL of TL buffer is added.

6) After adding 20 μL of proteinase K, the mixture is mixed well using a vortex.

7) Reaction is performed for 30 minutes in a constant-temperature water bath at 56° C.

8) After the reaction is completed, centrifugation is performed at 6,000×g or higher for about 10 seconds.

9) After adding 400 μL of TB buffer, the mixture is mixed well. Then, centrifugation is performed at 6,000×g or higher for about 10 seconds.

10) The reaction solution is added to a spin column mounted at a collection tube.

11) Centrifugation is performed at 6,000×g for 1 minute.

12) The filtrate that has passed through the column is discarded and a new collection tube is mounted.

13) After adding 700 μL of BW buffer, centrifugation is performed at 6,000×g for 1 minute.

14) The filtrate that has passed through the column is discarded and a new collection tube is mounted.

15) After adding 500 μL of NW buffer, centrifugation is performed at 13,500×g for 3 minutes.

16) The filtrate that has passed through the column is discarded and a new 1.5-mL tube is mounted.

17) After adding 200 μL of AE buffer or purified water, the column is allowed to stand at room temperature for 2 minutes.

18) Centrifugation is performed at 6,000×g for 1 minute.

19) The extracted genomic DNA can be directly used in PCR or may be stored at −20° C. for later use.

20) The extracted genomic DNA may be electrophoresed on 0.8% agarose gel and detected by UV.

C. Isolation of Genomic DNA from Paraffin-Embedded Sample

1) Paraffin-embedded sample is sliced to 20 μm thickness using a microtome or a surgical knife.

2) The sample is transferred to a 1.5-mL tube.

3) After adding 1.2 mL of xylene, the mixture is strongly mixed for 2 minutes using a vortex.

4) After centrifugation at 13,500×g for 5 minutes, the supernatant is removed.

5) After adding 1.2 mL of ethanol, the mixture is strongly mixed for 2 minutes using a vortex

6) After centrifugation at 13,500×g for 5 minutes, the supernatant is removed.

7) The procedure of 3)-5) is repeated to completely remove paraffin.

8) The tube holding the sample is allowed to stand at 37° C. for 15 minutes so that ethanol may be evaporated.

9) 200 μL of TL buffer is added to the sample in the tube.

10) After adding 20 μL of proteinase K, the mixture is mixed well using a vortex. 11) Reaction is performed in a constant-temperature water bath of 56° C. for 30 minutes.

12) After adding 400 μL of TB buffer, the mixture is mixed well. Centrifugation is performed at 6,000×g or higher for about 10 seconds.

13) The reaction solution is added to a spin column mounted at a collection tube.

14) Centrifugation is performed at 6,000×g for 1 minute.

15) The filtrate that has passed through the column is discarded and a new collection tube is mounted.

16) After adding 700 μL of BW buffer, centrifugation is performed at 6,000×g for 1 minute.

17) The filtrate that has passed through the column is discarded and a new collection tube is mounted.

18) After adding 500 μL of NW buffer, centrifugation is performed at 13,500×g for 3 minutes.

19) The filtrate that has passed through the column is discarded and a new 1.5-mL tube is mounted.

20) After adding 200 μL of AE buffer or purified water, the column is allowed stand at room temperature for 2 minutes.

21) Centrifugation is performed at 6,000×g for 1 minute.

22) The extracted genomic DNA can be directly used in PCR or may be stored at −20° C. for later use.

23) The extracted genomic DNA may be electrophoresed on 0.8% agarose gel and detected by UV.

### EXAMPLE 2

### Preparation of Standard and Control Samples

Plasmid DNA clone of HPV L1 gene which would serve as standard material in the following genotyping and analysis was prepared.

First, DNA was extracted from human cervical cancer cell and PCR product of HPV L1 gene was obtained. Second, PCR product of L1 gene of 42 types of HPV was obtained from Korea Food & Drug Administration (KFDA). Third, PCR product of HPV was obtained from cervical cancer tissues of 100 Korean women and cervical swab samples of 15,708 women. After genotyping HPV L1 gene by post-PCR sequencing, the PCR product was cloned to the pGEM-T Easy vector to acquire L1 clones for each HPV genotype. The clones were used as standard and control samples in the establishment of the reaction condition of the DNA chip of the present disclosure. The cloning was performed as follows.

1) The amplified PCR products of L1 gene were isolated using a gel recovery kit (Zymo Research, USA) on agarose gel and the concentration was measured using a spectrophotometer or on agarose gel.

2) pGEM-T Easy vector (Promega, A1360, USA) and 2x rapid ligation buffer that had been stored at −20° C. were melted and mixed slightly by shaking the tube slightly with fingers. After centrifugation at low speed, followed by mixing with insert DNA to be cloned with the following ratio, the mixture was added to a 0.5-mL tube for ligation reaction.

| | | Positive | Background |
|---|---|---|---|
| | Standard | control | control |
| 2x Rapid Ligation Buffer, T4 DNA | 5 μl | 5 μl | 5 μl |
| Ligase | | | |
| pGEM?-T Easy Vector (50 ng) | 1 μl | 1 μl | 1 μl |
| PCR product | X μl* | — | — |
| Control Insert DNA | — | 2 μl | — |
| T4 DNA Ligase(3 Wess units/μl) | 1 μl | 1 μl | 1 μl |
| Final volume | 10 μl | 10 μl | 10 μl |

*The ratio of the PCR product to the plasmid vector was adjusted to 3:1. That is, 50 ng of 3.0-kb vector was mixed with 12.4 ng of 0.25-kb PCR product or 22.5 ng of 0.45-kb PCR product, respectively.

3) After mixing the reaction solution well with a pipette, ligation was performed at room temperature for about an hour. When a large quantity of products were desired, the reaction was performed at 4° C. overnight.

4) Thus ligated sample was transformed with 50 μL of JM109 competent cell (=1×10⁸ cfu/μg DNA) stored at -70° C.

5) 2 μL of the ligated product was added to a 1.5-mL tube and 50 μL of the competent cell was added after thawing in ice bath immediately before the addition. After mixing well, reaction was carried out on ice for 20 minutes.

6) After applying heat shock for 45-50 seconds in a constant-temperature water bath at 42° C., the tube was immediately allowed to stand in ice bath for 2 minutes.

7) After adding 950 μL of SOC medium set to room temperature, the tube was incubated in a shaker at 37° C. for about 1.5 hours.

8) About 100 μL of the culture was applied on LB/ampicillin/IPTG/X-Gal plate. After reversing the plate and incubating in a shaker at 37° C. for about 16-24 hours, colony counting was carried out. Then, only the white colony was selected and cultured in 3 mL of LB/ampicillin broth. Plasmid DNA was miniprepared and it was checked whether the insert DNA was correctly inserted by PCR or using restriction enzymes. For more accurate analysis, all the clones obtained were analyzed using an automated base sequencer. Positive control clones are described in Table 1.

TABLE 1
Positive control clones

| | HPV | | | | |
|---|---|---|---|---|---|
| No. | subtype | Vector | Size (Kb) | Supplier | Note |
| 1 | 6B | pUC19 | 10.6 | ATCC | ATCC No. 45150 |
| 2 | 6 | pGEMTeasy | 3.85 | KFDA | |
| 3 | 10 | pGEMTeasy | 3.85 | KFDA | |
| 4 | 11 | pBR322 | 12.2 | ATCC | ATCC No. 45151 |
| 5 | 11 | pGEMTeasy | 3.85 | KFDA | |
| 6 | 16 | pBluescript | 10.9 | ATCC | ATCC No. 45113 |
| 7 | 16 | pGEMTeasy | 3.85 | KFDA | |
| 8 | 18 | pBR322 | 12.2 | ATCC | ATCC No. 45152 |
| 9 | 18 | pGEMTeasy | 3.85 | KFDA | |
| 10 | 26 | pGEMTeasy | 3.85 | KFDA | |
| 11 | 27 | pGEMTeasy | 3.85 | KFDA | |
| 12 | 30 | pGEMTeasy | 3.85 | KFDA | |
| 13 | 31 | pBR322 | 12.2 | ATCC | ATCC No. 65446 |
| 14 | 31 | pGEMTeasy | 3.85 | KFDA | |
| 15 | 32 | pGEMTeasy | 3.85 | KFDA | |
| 16 | 33 | pBR322 | 12.2 | BioMedLab | Institute Pasteur (France) |
| 17 | 33 | pGEMTeasy | 3.85 | KFDA | |
| 18 | 34 | pGEMTeasy | 3.5 | GoodGene | |
| 19 | 34 | pGEMTeasy | 3.85 | KFDA | |
| 20 | 35 | pT713 | 10.7 | BioMedLab | Diegen Co. (USA) |
| 21 | 35 | pGEMTeasy | 3.85 | KFDA | |
| 22 | 39 | pSP65 | 10.8 | BioMedLab | Institute Pasteur (France) |
| 23 | 39 | pGEMTeasy | 3.85 | KFDA | |
| 24 | 40 | pGEMTeasy | 3.5 | GoodGene | |
| 25 | 40 | pGEMTeasy | 3.85 | KFDA | |
| 26 | 42 | pBluescript | 10.9 | BioMedLab | National Institute of |
| | | | | | Infectious Disease (Japan) |
| 27 | 42 | pGEMTeasy | 3.85 | KFDA | |
| 28 | 43 | pGEMTeasy | 3.5 | GoodGene | |
| 29 | 43 | pGEMTeasy | 3.85 | KFDA | |
| 30 | 44 | pT713 | 10.6 | ATCC | ATCC No. 40353 |
| 31 | 44 | pGEMTeasy | 3.85 | KFDA | |
| 32 | 45 | pGEMTeasy | 3.6 | GoodGene | |
| 33 | 45 | pGEMTeasy | 3.85 | KFDA | |
| 34 | 51 | pGEMTeasy | 3.5 | GoodGene | |
| 35 | 51 | pGEMTeasy | 3.85 | KFDA | |
| 36 | 52 | pUC19 | 10.6 | ATCC | ATCC No. VRMC-29 |
| 37 | 52 | pGEMTeasy | 3.85 | KFDA | |
| 38 | 53 | pGEMTeasy | 3.85 | KFDA | |
| 39 | 54 | pGEMTeasy | 3.85 | KFDA | |
| 40 | 55 | pGEMTeasy | 3.85 | KFDA | |
| 41 | 56 | pT713 | 10.7 | ATCC | ATCC No. 40549 |
| 42 | 56 | pGEMTeasy | 3.85 | KFDA | |
| 43 | 57 | pGEMTeasy | 3.85 | KFDA | |
| 44 | 58 | plink322 | 11.6 | T. Matsukura, | National Institute of |
| | | | | | Infectious Disease (Japan) |
| 45 | 58 | pGEMTeasy | 3.85 | KFDA | |
| 46 | 59 | pUC9 | 10.6 | BioMedLab | National Institute of |
| | | | | | Infectious Disease (Japan) |
| 47 | 59 | pGEMTeasy | 3.85 | KFDA | |
| 48 | 61 | pGEMTeasy | 3.85 | KFDA | |
| 49 | 62 | pGEMTeasy | 3.85 | KFDA | |
| 50 | 66 | pBR322 | 12.2 | [symbol] | Institute Pasteur (France) |
| 51 | 66 | pGEMTeasy | 3.85 | KFDA | |
| 52 | 67 | pGEMTeasy | 3.85 | KFDA | |
| 53 | 68 | pGEMTeasy | 3.5 | [symbol] | |
| 54 | 68 | pGEMTeasy | 3.85 | KFDA | |
| 55 | 69 | pBluescript | 10.8 | T. Matsukura, | National Institute of |
| | | | | | Infectious Disease (Japan) |
| 56 | 69 | pGEMTeasy | 3.85 | KFDA | |
| 57 | 70 | pGEMTeasy | 3.85 | KFDA | |
| 58 | 72 | pGEMTeasy | 3.85 | KFDA | |
| 59 | 73 | pGEMTeasy | 3.85 | KFDA | |
| 60 | 81 | pGEMTeasy | 3.85 | KFDA | |
| 61 | 82 | pGEMTeasy | 3.85 | KFDA | |
| 62 | 83 | pGEMTeasy | 3.85 | [symbol] | |
| 63 | 84 | pGEMTeasy | 3.85 | KFDA | |
| 64 | 90 | pGEMTeasy | 3.85 | KFDA | |
| 65 | 91 | pGEMTeasy | 3.85 | KFDA | |

### EXAMPLE 3

### PCR Amplification

HPV L1 gene and human beta-actin gene as internal control gene were amplified to investigate the genotype of HPV.

For PCR amplification, oligonucleotide primers were selected and designed first. The primers include MY11, GP6-1 and GP6+primers (SEQ ID NOS 1-3) for detecting the HPV L1 gene and ACTB F (forward) and ACTB R (reverse) primers of human beta-actin gene for confirming DNA extraction and. PCR efficiency. The GP6-1, ACTBF and ACTBR primers were designed by the inventors and the other primers were selected from previously known primers. The PCR product of the HPV L1 gene is 185 by in length and that of the beta-actin gene is 102 by long. The base sequence of the PCR primers for each gene is described in Table 2.

TABLE 2
Primers for PCR

| | | | | | TM | GC |
|---|---|---|---|---|---|---|
| No | Gene | Name | Sequence (5′->3′) | Mer | (° C.) | % |
| SEQ ID | ACTB | ACTB F | GCA CCA CAC CTT CTA CAA | 20 | 46.8 | 45 |
| NO 1 | Primer | | TGA | | | |
| SEQ ID | | ACTB R | Cy5-GTC ATC TTC TCG CGG | 21 | 56.6 | 48 |
| NO 2 | | | TTG GC | | | |
| SEQ ID | HPV | L1 | GCM CAG GGW CAT AAY AAT | 20 | 66 | 50 |
| NO 3 | Primer | | GG | | | |
| SEQ ID | | L2 | Cy5-AATAAACTGTAAATCATA | 24 | 47.7 | 25 |
| NO 4 | | | TTCCTC | | | |
| SEQ ID | | GP6+ | Cy5-GAAAAATAAACTGTAAAT | 24 | 47 | 25 |
| NO 5 | | | CATATTC | | | |

(In the base sequences, M is A or C, W is A or T and Y is C or T.)

Optimal condition for duplex PCR was established and PCR of HPV L1 and human beta-actin genes was performed using the DNA isolated in Example 2 as template. Details are as follows.

A PCR reaction solution for detecting HPV infection was prepared by adding 1 μL (10 pmol) of MY11 primer, 1 μL (8 pmol) of GP6-1 primer, 1 μL (8 pmol) of GP6+ primer, 1 μL (5 pmol) of ACTBF primer and 1 μL (5 pmol) of ACTBR primer to 15 μL of SuperTaq Plus pre-mix (10× buffer 2.5 μL, 10 mM MgCl₂ 3.75 μL, 10 mM dNTP 0.5 μL, Taq polymerase 0.5 μL) purchased from Super Bio (Seoul, Korea), as described in Table 2. 4 μL (150 ng/μL) of template DNA of the sample was added and the total volume of the reaction solution was adjusted to 30 μL by adding distilled water.

For Duplex PCR, the reaction solution containing each primer was predenatured at 95° C. for 5 minutes and 40 cycles of 95° C. for 30 seconds, 50° C. for 30 seconds and 72° C. for 30 seconds were repeated. Then, extension was carried out at 72° C. for 5 minutes.

The result is shown in FIG. 2. It was confirmed that the duplex PCR condition was established adequately and PCR was carried out successfully for the cervical swab sample and paraffin-embedded cervical cancer tissue.

The PCR result for HPV L1 gene for 15,708 cervical clinical samples is given in Table 3. 7,371 samples exhibited positive results. Particularly, HPV-11 or HPV-56 which could not be amplified by the GP6-1 primer could be amplified by the GP6+ primer. Also, non-specific PCR that may occur when the DNA concentration is too low could be overcome through the duplex PCR. Based on this result, the HPV genotype DNA chip of the present disclosure could be designed.

TABLE 3
PCR result for HPV for cervical cell samples from Koreans
Age

| Infection type | 10s | 20s | 30s | 40s | 50s | 60s | 70s | 80s | NA | Total |
|---|---|---|---|---|---|---|---|---|---|---|
| Single | 17 | 1,017 | 1,196 | 1,115 | 420 | 91 | 22 | 1 | 792 | 4,671 |
| Mixed (2) | 20 | 567 | 578 | 471 | 169 | 37 | 11 | | 377 | 2,230 |
| Mixed (3) | 3 | 121 | 106 | 79 | 35 | 6 | 1 | | 82 | 433 |
| Mixed (4)) | 1 | 8 | 14 | 4 | 4 | | | | 6 | 37 |
| Negative total | 16 | 1,270 | 2,217 | 2,236 | 861 | 209 | 28 | 1 | 1,499 | 8,337 |
| Positive total | 41 | 1713 | 1894 | 1669 | 628 | 134 | 34 | 1 | 1257 | 7371 |
| Positive (%) | 71.93 | 57.43 | 46.07 | 42.74 | 42.18 | 39.07 | 54.84 | 50 | 45.61 | 46.93 |
| Negative (%) | 28.07 | 42.57 | 53.93 | 57.26 | 57.82 | 60.93 | 45.16 | 50 | 54.39 | 53.07 |
| Total | 57 | 2,983 | 4,111 | 3,905 | 1,489 | 343 | 62 | 2 | 2,756 | 15,708 |

Non-specific chip reaction that may occur in single PCR when the DNA concentration of HPV-negative sample is low could be overcome through the duplex PCR according to the present disclosure. For comparison, the product of single PCR performed using the existing HPV DNA genotyping chip (L1 gene probe & HBB gene probe) for 43 types of HPV and with the product of duplex PCR performed according to the present disclosure were respectively subjected to chip reactions and the chip images were compared after scanning (see FIG. 4). As seen from FIG. 4, the non-specific reaction observed in single PCR disappeared in the duplex PCR product. Accordingly, it can be seen that duplex PCR is much more effective than single PCR.

### EXAMPLE 4

### Sequencing Analysis and Establishment of Database

After the PCR in Example 3, automated sequencing analysis of the PCR product was carried out to analyze the base sequence of HPV L1 and a database was built based on the result. In addition, the clinical DNA samples whose HPV genotype was confirmed were stored and used for analysis of accuracy of the DNA chip of the present disclosure. The sequencing reaction was carried out using the ABI 3130XL sequencer and BigDye Terminator v 2.0 according to the known method.

First, 100 paraffin-embedded cervical cancer tissue samples and 50 normal cervical tissue samples were subjected to HPV genotyping using the DNA chip of the present disclosure and by sequencing. As a result, high-risk type HPV was found in 98 out of the 100 cervical cancer tissue samples. In contrast, no high-risk type HPV was found in the normal cervical tissue samples (Table 4).

TABLE 4
HPV genotyping result for 100 CIN samples

| HPV type | PCR-sequencing of L1 | HPV DNA Chip analysis |
|---|---|---|
| 16 | 37 | 42 |
| 58 | 16 | 18 |
| 31 | 13 | 14 |
| 18 | 5 | 5 |
| 35 | 4 | 5 |
| 33 | 5 | 5 |
| 52 | 3 | 3 |
| 34 | 2 | 2 |
| 26 | 1 | 1 |
| 39 | 1 | 1 |
| 56 | 1 | 1 |
| 53 | 1 | 1 |
| Mixed types | 0 | 7* |
| Accurate Detection No | 89 (90.8) | 98 (100) |
| (%) | | |

That is to say, high-risk type HPV was found in 98 out of the 100 cervical cancer tissue samples (98%) as a result of the DNA chip analysis. Among them, 42 samples were HPV-16, 18 samples were HPV-58, 14 samples were HPV-31, 5 samples were HPV-18, 5 samples were HPV-35 and 5 samples were HPV-33. These 7 types accounted for 98%. In contrast to the DNA chip of the present disclosure, only 89 samples (90.8%) could be identified by PCR sequencing. Especially, mixed infection could not be detected with PCR sequencing. This result indicates that the HPV DNA chip of the present disclosure is useful in predicting the pathological condition of the cervix and, particularly, in screening of cervical cancer and carcinoma in situ. Further, it was confirmed again that the mixed HPV infection undetectable with sequencing can be accurately detected.

### EXAMPLE 5

### Design of Probes of DNA Chip

In order to design oligonucleotide probes to be positioned on the DNA chip, the huge database containing information regarding the base sequence of L1 gene of the 98 types of HPV identified from the benign and malignant cervical samples of Korean women by post-PCR sequencing in Examples 4-5 and the US HPV database were analyzed. Also, intra-variant base sequences present in each gene were analyzed according to HPV genotype and frequency thereof for each human race. As a result, 43 types of genital type HPV invading the cervix were selected and oligonucleotide probes for genotyping them were designed (Table 5).

The oligonucleotide probes were designed as genotype-specific probes capable of specifically binding to the HPV L1 gene DNA of the 43 types of HPV.

Based on (1) HPV database of the US National Center for Biotechnology Information (NCBI), (2) US Los Alamos HPV database and (3) the database of the 45 types of HPV detected from the cervical samples of Korean women in Example 4, genomic DNA base sequences of a total of 79 types of HPV: HPV-1a, -2a, -3, -4, -5, -6b, -7, -8, -9, -10, -11, -12, -13, -15, -16, -16r, -17, -18, -19, -20, -21, -22, -23, -24, -25, -26, -27, -28, -29, -30, -31, -32, -33, -34, -35, -35h, -36, -37, -38, -39, -40, -41, -42, -44, -45, -47, -48, -49, -50, -51, -52, -53, -54, -55, -56, -57, -58, -59, -60, -61, -63, -65, -66, -67, -68a, -68b, -70, -72, -73, -75, -76, -77, -80, -90, -91, MM4(82), MM7(83), MM8(84) and CP8304 were obtained. Based on the obtained DNA sequences, phylogenetic tree was drawn using the computer program DNASTAR (MegAlign™ 5, DNASTAR Inc.) according to the ClustalW method (pairwise alignment and multiple sequence alignment). After screening genotype-specific base sequences for each group, genotype-specific probes were designed using the computer program Primer Premier 5 (Premier Biosoft International Co.).

110 genotype-specific oligonucleotide probes were designed first by setting probe lengths to 20±2 and 18±2 bp. In the HPV DNA chip and diagnosis kit according to the present disclosure, the DNA probes target a total of 43 HPV L1 genes including 14 high-risk type HPV L1 genes, 22 low-risk type HPV L1 genes and 7 moderate-risk type HPV L1 genes. The high-risk type HPVs include HPV-16, HPV-18, HPV-31, HPV-33, HPV-35, HPV-39, HPV-45, HPV-51, HPV-52 HPV-56, HPV-58, HPV-59, HPV-68, HPV-82, HPV-26, HPV-53, HPV-66, HPV-67, HPV-69, HPV-70 and HPV-73, and the low-risk type HPVs include HPV-6, HPV-11, HPV-34, HPV-40, HPV-42, HPV-43, HPV-44, HPV-54, HPV-55 HPV-61, HPV-62, HPV-72, HPV-81, HPV-90, HPV-10, HPV-27, HPV-30, HPV-32, HPV-57, HPV-83, HPV-84 and HPV-91.

Virtual binding ability of the 110 probes designed above to the 76 different types of HPV was analyzed using the computer program Amplify 1.2 (University of Wisconsin). Probes for HPV-16, HPV-58, HPV-31 and HPV-33 that are common to Koreas and closely related to cervical cancer were designed. Next, probes capable of specifically binding to HPV-18, HPV-35, HPV-39, HPV-45, HPV-51, HPV-52 HPV-56, HPV-58, HPV-59, HPV-68, HPV-82, HPV-26, HPV-53, HPV-66, HPV-67, HPV-69, HPV-70, HPV-73, HPV-6, HPV-11, HPV-34, HPV-40, HPV-42, HPV-43, HPV-44, HPV-54, HPV-55 HPV-61, HPV-62, HPV-72, HPV-81, HPV-90, HPV-10, HPV-27, HPV-30, HPV-32, HPV-57, HPV-83, HPV-84 and HPV91 were selected. The name, SEQ ID NO and type of the linear oligonucleotide probes are summarized in Table 5.

TABLE 5
Linear oligonucleotide probes

| | | | | TM | GC |
|---|---|---|---|---|---|
| No | Name | Sequence (5′->3′) | bp | (° C.) | % |
| Sequence | HPV 6 | GCATCCGTAACTACATCTTCCA | 22 | 55.6 | 45 |
| ID No. 6 | P-1 | | | | |
| Sequence | HPV 6 | TGTGCATCCGTAACTACATCTTCC | 25 | 77 | 44 |
| ID No. 7 | P-2 | A | | | |
| Sequence | HPV 7 | ACACCAACACCATATGACAAT | 22 | 51.6 | 36 |
| ID No. 8 | P-1 | | | | |
| Sequence | HPV 7 | CGCCCACACCAACACCATATGAC | 27 | 80 | 50 |
| ID No. 9 | P-2 | AATA | | | |
| Sequence | HPV 10 | CCTCCCCTGCCACTACG | 18 | 60.2 | 72 |
| ID No. 10 | P-1 | | | | |
| Sequence | HPV 10 | TCTGAGCCTCCCCTGCCACTACG | 23 | 79 | 65 |
| ID No. 11 | P-2 | | | | |
| Sequence | HPV 11 | ATTTGCTGGGGAAACCAC | 18 | 54.4 | 50 |
| ID No. 12 | P-1 | | | | |
| Sequence | HPV 11 | TATTTGCTGGGGAAACCACT | 20 | 72 | 45 |
| ID No. 13 | P-2 | | | | |
| Sequence | HPV 16 | TGCCATATCTACTTCAGAAACT | 22 | 49.9 | 36 |
| ID No. 14 | P-1 | | | | |
| Sequence | HPV 16 | TGTGCTGCCATATCTACTTCAGAA | 27 | 79 | 41 |
| ID No. 15 | P-2 | ACT | | | |
| Sequence | HPV 18 | TCTACACAGTCTCCGTACCTG | 21 | 51.5 | 52 |
| ID No. 16 | P-1 | | | | |
| Sequence | HPV 18 | AATATGTCTACACAGTCTCCGTAC | 27 | 74 | 44 |
| ID No. 17 | P-2 | CTG | | | |
| Sequence | HPV 26 | ATTATCTGCAGCATCTGCATCC | 22 | 57.9 | 45 |
| ID No. 18 | P-1 | | | | |
| Sequence | HPV 26 | TAGTACATTATCTGCAGCATCTGC | 28 | 76 | 43 |
| ID No. 19 | P-2 | ATCC | | | |
| Sequence | HPV 27 | CAGCTGAGGTGTCTGATAATACT | 26 | 54.2 | 38 |
| ID No. 20 | P-1 | AAT | | | |
| Sequence | HPV 27 | GTGTGCAGCTGAGGTGTCTGATA | 31 | 80 | 42 |
| ID No. 21 | P-2 | ATACTAAT | | | |
| Sequence | HPV 30 | AACCACACAAACGTTATCCA | 20 | 52.6 | 40 |
| ID No. 22 | P-1 | | | | |
| Sequence | HPV 30 | ATCTGCAACCACACAAACGTTAT | 26 | 78 | 42 |
| ID No. 23 | P-2 | CCA | | | |
| Sequence | HPV 31 | CTGCAATTGCAAACAGTGATAC | 22 | 54.7 | 41 |
| ID No. 24 | P-1 | | | | |
| Sequence | HPV 31 | TTTGTGCTGCAATTGCAAACAGTG | 25 | 78 | 44 |
| ID No. 25 | P-2 | ATAC | | | |
| Sequence | HPV 32 | GACACATACAAGTCTACTAACTTT | 25 | 46.4 | 32 |
| ID No. 26 | P-1 | A | | | |
| Sequence | HPV 32 | ACTGAAGACACATACAAGTCTAC | 31 | 76 | 32 |
| ID No. 27 | P-2 | TAACTTTA | | | |
| Sequence | HPV 33 | GCACACAAGTAACTAGTGACAGT | 25 | 51.7 | 44 |
| ID No. 28 | P-1 | AC | | | |
| Sequence | HPV 33 | CTTTATGCACACAAGTAACTAGT | 31 | 75 | 39 |
| ID No. 29 | P-2 | GACAGTAC | | | |
| Sequence | HPV 34 | CCACAAGTACAACTGCACC | 19 | 48 | 52.6 |
| ID No. 30 | P-1 | | | | |
| Sequence | HPV 34 | CAATCCACAAGTACAACTGCACC | 23 | 73 | 48 |
| ID No. 31 | P-2 | | | | |
| Sequence | HPV 35 | TCTGCTGTGTCTTCTAGTGACAGT | 25 | 52.6 | 44 |
| ID No. 32 | P-1 | A | | | |
| Sequence | HPV 35 | TGTGTTCTGCTGTGTCTTCTAGTG | 30 | 77 | 43 |
| ID No. 33 | P-2 | ACAGTA | | | |
| Sequence | HPV 39 | ACCTCTATAGAGTCTTCCATACCT | 29 | 55.7 | 41 |
| ID No. 34 | P-1 | TCTAC | | | |
| Sequence | HPV 39 | TTATCTACCTCTATAGAGTCTTCC | 35 | 76 | 37 |
| ID No. 35 | P-2 | ATACCTTCTAC | | | |
| Sequence | HPV 40 | AGTCCCCCACACCAAC | 16 | 50 | 63 |
| ID No. 36 | P-1 | | | | |
| Sequence | HPV 40 | CCACACAGTCCCCCACACCAAC | 22 | 80 | 64 |
| ID No. 37 | P-2 | | | | |
| Sequence | HPV 42 | CACTGCAACATCTGGTGA | 18 | 50.1 | 50 |
| ID No. 38 | P-1 | | | | |
| Sequence | HPV 42 | GTGTGCCACTGCAACATCTGGTG | 24 | 77 | 54 |
| ID No. 39 | P-2 | A | | | |
| Sequence | HPV 43 | GCCCAGTACATATGACAATGCA | 22 | 54.7 | 45.4 |
| ID No. 40 | P-1 | | | | |
| Sequence | HPV 43 | TACTGTGCCCAGTACATATGACA | 28 | 78 | 43 |
| ID No. 41 | P-2 | ATGCA | | | |
| Sequence | HPV 44 | TACACAGTCCCCTCCGTC | 18 | 49.7 | 61.1 |
| ID No. 42 | P-1 | | | | |
| Sequence | HPV 44 | TGCCACTACACAGTCCCCTCCGTC | 24 | 79 | 63 |
| ID No. 43 | P-2 | | | | |
| Sequence | HPV 45 | CACAAAATCCTGTGCCAAG | 19 | 53.7 | 47 |
| ID No. 44 | P-1 | | | | |
| Sequence | HPV 45 | CCTCTACACAAAATCCTGTGCCA | 25 | 74 | 48 |
| ID No. 45 | P-2 | AG | | | |
| Sequence | HPV 51 | GGTTTCCCCAACATTTACTC | 20 | 52.3 | 45 |
| ID No. 46 | P-1 | | | | |
| Sequence | HPV 51 | TGCGGTTTCCCCAACATTTACTC | 23 | 78 | 48 |
| ID No. 47 | P-2 | | | | |
| Sequence | HPV 52 | GCTGAGGTTAAAAAGGAAAGCA | 22 | 56.6 | 41 |
| ID No. 48 | P-1 | | | | |
| Sequence | HPV 52 | CTTTATGTGCTGAGGTTAAAAAG | 30 | 77 | 37 |
| ID No. 49 | P-2 | GAAAGCA | | | |
| Sequence | HPV 53 | CGCAACCACACAGTCTATGTCTA | 23 | 56.6 | 48 |
| ID No. 50 | P-1 | | | | |
| Sequence | HPV 53 | CTCTTTCCGCAACCACACAGTCTA | 30 | 79 | 47 |
| ID No. 51 | P-2 | TGTCTA | | | |
| Sequence | HPV 54 | TACAGCATCCACGCAGG | 17 | 53.3 | 59 |
| ID No. 52 | P-1 | | | | |
| Sequence | HPV 54 | GTGTGCTACAGCATCCACGCAGG | 23 | 77 | 61 |
| ID No. 53 | P-2 | | | | |
| Sequence | HPV 55 | CTACAACTCAGTCTCCATCTACAA | 24 | 51.9 | 42 |
| ID No. 54 | P-1 | | | | |
| Sequence | HPV 55 | GTGCTGCTACAACTCAGTCTCCAT | 30 | 79 | 47 |
| ID No. 55 | P-2 | CTACAA | | | |
| Sequence | HPV 56 | GACTATTAGTACTGCTACAGAAC | 34 | 55.1 | 32.4 |
| ID No. 56 | P-1 | AGTTAAGTAAA | | | |
| Sequence | HPV 56 | TACTGCTACAGAACAGTTAAGTA | 25 | 72 | 32 |
| ID No. 57 | P-2 | AA | | | |
| Sequence | HPV 57 | CCACTGTAACCACAGAAACTAAT | 24 | 53.3 | 38 |
| ID No. 58 | P-1 | T | | | |
| Sequence | HPV 57 | GTGTGCCACTGTAACCACAGAAA | 29 | 80 | 41 |
| ID No. 59 | P-2 | CTAATT | | | |
| Sequence | HPV 58 | TGCACTGAAGTAACTAAGGAAGG | 23 | 54.4 | 43 |
| ID No. 60 | P-1 | | | | |
| Sequence | HPV 58 | GACATTATGCACTGAAGTAACTA | 30 | 76 | 40 |
| ID No. 61 | P-2 | AGGAAGG | | | |
| Sequence | HPV 59 | TCTATTCCTAATGTATACACACCT | 29 | 56.5 | 38 |
| ID No. 62 | P-1 | ACCAG | | | |
| Sequence | HPV 59 | CTTCTTCTATTCCTAATGTATACA | 34 | 74 | 38 |
| ID No. 63 | P-2 | CACCTACCAG | | | |
| Sequence | HPV 61 | TGCTACATCCCCCCCTGTAT | 20 | 57.8 | 55 |
| ID No. 64 | P-1 | | | | |
| Sequence | HPV 61 | TTTGTACTGCTACATCCCCCCCTG | 27 | 77 | 48 |
| ID No. 65 | P-2 | TAT | | | |
| Sequence | HPV 62 | ACTATTTGTACCGCCTCCAC | 20 | 53 | 50 |
| ID No. 66 | P-1 | | | | |
| Sequence | HPV 62 | ACTATTTGTACCGCCTCCACTGCT | 25 | 78 | 52 |
| ID No. 67 | P-2 | G | | | |
| Sequence | HPV 66 | AATGCAGCTAAAAGCACATTAAC | 26 | 56.9 | 31 |
| ID No. 68 | P-1 | TAA | | | |
| Sequence | HPV 66 | CTATTAATGCAGCTAAAAGCACA | 31 | 75 | 29 |
| ID No. 69 | P-2 | TTAACTAA | | | |
| Sequence | HPV 67 | AAAATCAGAGGCTACATACAAAA | 23 | 51.8 | 30 |
| ID No. 70 | P-1 | | | | |
| Sequence | HPV 67 | CTGAGGAAAAATCAGAGGCTACA | 30 | 77 | 37 |
| ID No. 71 | P-2 | TACAAAA | | | |
| Sequence | HPV 68b | CTACTACTACTGAATCAGCTGTAC | 31 | 54.9 | 35.5 |
| ID No. 72 | P-1 | CAAATAT | | | |
| Sequence | HPV 68b | TTTGTCTACTACTACTGAATCAGC | 36 | 79 | 33 |
| ID No. 73 | P-2 | TGTACCAAATAT | | | |
| Sequence | HPV | CAGACTCTACTGTACCAGCTG | 23 | 53.2 | 52 |
| ID No. 74 | 68aP-1 | | | | |
| Sequence | HPV | TACAGACTCTACTGTACCAGCTG | 23 | 71 | 48 |
| ID No. 75 | 68aP-2 | | | | |
| Sequence | HPV | TACTACAGACTCTACTGTACCAGC | 26 | 72 | 46 |
| ID No. 76 | 68aP-3 | TG | | | |
| Sequence | HPV | CAGACTCTACTGTACCAGCTGTG | 23 | 73 | 52 |
| ID No. 77 | 68aP-4 | | | | |
| Sequence | HPV 69 | CACAATCTGCATCTGCCACTTTTA | 25 | 61 | 40 |
| ID No. 78 | P-1 | A | | | |
| Sequence | HPV 69 | GTATCTGCACAATCTGCATCTGCC | 32 | 82 | 41 |
| ID No. 79 | P-2 | ACTTTTAA | | | |
| Sequence | HPV 70 | CCGAAACGGCCATACCT | 17 | 55.5 | 59 |
| ID No. 80 | P-1 | | | | |
| Sequence | HPV 70 | CTGCACCGAAACGGCCATACCT | 22 | 80 | 59 |
| ID No. 81 | P-2 | | | | |
| Sequence | HPV 72 | CACAGCGTCCTCTGTATCAGA | 21 | 55.1 | 52 |
| ID No. 82 | P-1 | | | | |
| Sequence | HPV 72 | TACTGCCACAGCGTCCTCTGTATC | 27 | 80 | 52 |
| ID No. 83 | P-2 | AGA | | | |
| Sequence | HPV 73 | AGGTACACAGGCTAGTAGCTCTA | 27 | 54.4 | 48 |
| ID No. 84 | P-1 | CTAC | | | |
| Sequence | HPV 73 | TGTAGGTACACAGGCTAGTAGCT | 30 | 77 | 47 |
| ID No. 85 | P-2 | CTACTAC | | | |
| Sequence | HPV 81 | GCTACATCTGCTGCTGCAGA | 20 | 56.5 | 55 |
| ID No. 86 | P-1 | | | | |
| Sequence | HPV 81 | TTTGCACAGCTACATCTGCTGCTG | 28 | 79 | 50 |
| ID No. 87 | P-2 | CAGA | | | |
| Sequence | HPV 82 | CTCCAGCAAACTTTAAGCA | 19 | 50.5 | 42 |
| ID No. 88 | P-1 | | | | |
| Sequence | HPV 82 | CTCCAGCAAACTTTAAGCAATAC | 24 | 74 | 38 |
| ID No. 89 | P-2 | A | | | |
| Sequence | HPV 83 | TGCTGCTACACAGGCTAATGA | 27 | 55.9 | 48 |
| ID No. 90 | P-1 | | | | |
| Sequence | HPV 83 | TCAGCTGCTGCTACACAGGCTAA | 26 | 80 | 50 |
| ID No. 91 | P-2 | TGA | | | |
| Sequence | HPV 84 | ACCGAATCAGAATATAAACCTAC | 24 | 57.7 | 33 |
| ID No. 92 | P-1 | CAAT | | | |
| Sequence | HPV 84 | CAACACCGAATCAGAATATAAAC | 31 | 75 | 35 |
| ID No. 93 | P-2 | CTACCAAT | | | |
| Sequence | HPV 90 | ACAAACACCCTCTGACACATACA | 23 | 55.7 | 43 |
| ID No. 94 | P-1 | | | | |
| Sequence | HPV 90 | CCACACAAACACCCTCTGACACA | 27 | 78 | 48 |
| ID No. 95 | P-2 | TACA | | | |
| Sequence | HPV 91 | TCTGTGCTACCTACTACATATGAC | 28 | 57.3 | 39 |
| ID No. 96 | P-1 | AACA | | | |
| Sequence | HPV 91 | ACTGAGTCTGTGCTACCTACTACA | 34 | 77 | 41 |
| ID No. 97 | P-2 | TATGACAACA | | | |
| Sequence | HPV | TTGTTGGGDTAATCAGTTGTTTGT | 30 | 61.2 | 34 |
| ID No. 98 | U P-1 | TACTGT | | | |
| Sequence | HPV | TTTGTTACTGTTGTAGATACTACT | 32 | 74 | 38 |
| ID No. 99 | U P-2 | CGCAGTAC | | | |
| Sequence | HPV | TTGTTGGGDTAATCARTTRTTTGT | 30 | 65 | 32 |
| ID No. 100 | U P-3 | TACDGT | | | |
| Sequence | HPV | TTTKTTACHGTKGTDGATACYAC | 23 | 51 | 36 |
| ID No. 101 | U P-4 | | | | |
| Sequence | HPV | TGTTTRTTACTGTTGTDGAYACYA | 25 | 60 | 35 |
| ID No. 102 | U P-5 | C | | | |
| Sequence | HPV | TATTTGTAACTGTTGTGGATACCA | 25 | 71 | 36 |
| ID No. 103 | U P-6 | C | | | |
| Sequence | HPV | TTTRTTACTGTTGTDGAYACYAC | 23 | 55 | 34 |
| ID No. 104 | U P-7 | | | | |
| Sequence | HPV | TATTTRTTACTGTTGTDGAYACYA | 25 | 57 | 31 |
| ID No. 105 | U P-8 | C | | | |
| Sequence | ACTB-1P | ACCCCGTGCTGCTGACCGAGGC | 22 | 72.2 | 73 |
| ID No. 106 | | | | | |
| Sequence | ACTB-2P | CACCCCGTGCTGCTGACCG | 19 | 66.9 | 74 |
| ID No. 107 | | | | | |
| Sequence | ACTB-3P | CACCCCGTGCTGCTGACCGAGGC | 23 | 83 | 74 |
| ID No. 108 | | | | | |
| Sequence | ACTB-4P | GCTGCGTGTGGCTCCCGAGG | 20 | 78 | 75 |
| ID No. 109 | | | | | |

(In the base sequences, D is G, A or T, K is G or T and Y is C or T.)

### EXAMPLE 6

### Designing of D-Shaped Probe

A d-shaped oligonucleotide probe having a stem structure was designed. The d-shaped probe of the present disclosure comprises, in 5′→3′ direction and from left top to right top, (1) a left stem part, (2) a linker part, (3) a right stem part and (4) a right probe part (see FIG. 22). The base sequence of the d-shaped probe for the HPV L1 gene and the human beta-actin gene is shown in Table 6.

(1) Stem Part

For the d-shaped probe of the present disclosure to be structurally stable, a stem part supporting the probe should be adequately designed. The stem part comprises oligonucleotides having complementary sequences bound to each other. For strong binding, the stem part should comprise C and G bases at least in half and T or A base may be inserted therebetween. The stem part may comprise a naturally occurring telomere. At the end of the chromosome of an eukaryotic organism, a telomere consisting of repetitive base sequences exists. The sequence is TTAGGG, TTTAGGG or T1-3(T/A)G3—for mammals including human and TTGGGG or TTTTGGGG for other organisms (Balagurumoothy P, Brahmachari S K, Mohnaty D, Bansal M and Sasisekharan V. Hairpin and parallel quartet structures for telomeric sequences. Nucleic Acids Research. 1992; 20(15): 4061-4067; Balagurumoothy P and Brahmachari S K. Structure and stability of human telomeric sequence. Journal of Biochemistry. 1994; 269(34): 21858-21869). Accordingly, the stem part of the d-shaped probe of the present disclosure may comprise at least one repeating base selected from the following on one strand.

e.g.)

| | |
|---|---|
| 1. | TTGGG |
| 2. | TAGGG |
| 3. | TTGGGG |
| 4. | TTTGGG |
| 5. | TTAGGG |
| 6. | TTTGGGG |
| 7. | TTTAGGG |
| 8. | TTTTGGGG |
| 9. | TTTAGGGG |

That is to say, 5-9 oligonucleotides may bind complementarily, and the number of the oligonucleotides can be increased further. In terms of cost and efficiency, the human telomere comprising the nucleotide sequence TTAGGG-AATCCC may be used as the repeating unit. However, the length can be changed variously.

(2) Linker Part

In the present disclosure, amino-modified dideoxythymidine (internal amino modifier CndT; iAmMCnT) with n ranging from 3 to 60 is inserted. In terms of economic efficiency, short iAmMC6T having 6 carbons may be used. At the 5′-terminal of iAmMC6dT, the modified C6 amine linker of the left stem part binds with the aldehyde group coated on the glass slide surface. The base A of the 3′-terminal binds with the base T of the 5′-terminal of the right stem part. The d-shaped probe may be fixed on a chip via binding to the ribose of the iAmMC6dT.

(3) Right Probe Part

The right probe part is designed to be complementary to the target gene to be detected. Any base sequence is possible, but the oligonucleotide sequence and length of the right probe part should be adequately designed. The probe part should be selected such that a secondary structure is not formed. The right probe part may be usually about 15-75 by in length, but the length may be increased to about 150 by or decreased to shorter than 15 by depending on situations. If the sample is a PCR product as in the present disclosure and if it is desired not only to detect HPV infection but also to analyze the accurate type and subtype thereof, the probe length may be about 20 by and it is designed such that the difference in at least three nucleotides at the center portion is discernible.

TABLE 6
Base sequence of d-shaped oligonucleotide probe

| No | Name | Sequence (5′->3′) | bp |
|---|---|---|---|
| Sequence | HPV 6 | CCCTAACCCTAA--iAmMC6T-TTAGGGTTAG | 46 |
| ID No. | DP-1 | GGGCATCCGTAACTACATCTTCCA | |
| 110 | | | |
| Sequence | HPV 6 | CCCTAACCCTAA--iAmMC6T-TTAGGGTTAG | 49 |
| ID No. | DP-2 | GGTGTGCATCCGTAACTACATCTTCCA | |
| 111 | | | |
| Sequence | HPV 7 | CCCTAACCCTAA--iAmMC6T-TTAGGGTTAG | 46 |
| ID No. | DP-1 | GGACACCAACACCATATGACAAT | |
| 112 | | | |
| Sequence | HPV 7 | CCCTAACCCTAA--iAmMC6T-TTAGGGTTAG | 51 |
| ID No. | DP-2 | GGCGCCCACACCAACACCATATGACAATA | |
| 113 | | | |
| Sequence | HPV 10 | CCCTAACCCTAA--iAmMC6T-TTAGGGTTAG | 42 |
| ID No. | DP-1 | GGCCTCCCCTGCCACTACG | |
| 114 | | | |
| Sequence | HPV 10 | CCCTAACCCTAA--iAmMC6T-TTAGGGTTAG | 47 |
| ID No. | DP-2 | GGTCTGAGCCTCCCCTGCCACTACG | |
| 115 | | | |
| Sequence | HPV 11 | CCCTAACCCTAA--iAmMC6T-TTAGGGTTAG | 42 |
| ID No. | DP-1 | GGATTTGCTGGGGAAACCAC | |
| 116 | | | |
| Sequence | HPV 11 | CCCTAACCCTAA--iAmMC6T-TTAGGGTTAG | 44 |
| ID No. | DP-2 | GGTATTTGCTGGGGAAACCACT | |
| 117 | | | |
| Sequence | HPV 16 | CCCTAACCCTAA--iAmMC6T-TTAGGGTTAG | 46 |
| ID No. | DP-1 | GGTGCCATATCTACTTCAGAAACT | |
| 118 | | | |
| Sequence | HPV 16 | CCCTAACCCTAA--iAmMC6T-TTAGGGTTAG | 51 |
| ID No. | DP-2 | GGTGTGCTGCCATATCTACTTCAGAAACT | |
| 119 | | | |
| Sequence | HPV 18 | CCCTAACCCTAA--iAmMC6T-TTAGGGTTAG | 45 |
| ID No. | DP-1 | GGTCTACACAGTCTCCGTACCTG | |
| 120 | | | |
| Sequence | HPV 18 | CCCTAACCCTAA--iAmMC6T-TTAGGGTTAG | 51 |
| ID No. | DP-2 | GGAATATGTCTACACAGTCTCCGTACCTG | |
| 121 | | | |
| Sequence | HPV 26 | CCCTAACCCTAA--iAmMC6T-TTAGGGTTAG | 46 |
| ID No. | DP-1 | GGATTATCTGCAGCATCTGCATCC | |
| 122 | | | |
| Sequence | HPV 26 | CCCTAACCCTAA--iAmMC6T-TTAGGGTTAG | 52 |
| ID No. | DP-2 | GGTAGTACATTATCTGCAGCATCTGCATCC | |
| 123 | | | |
| Sequence | HPV 27 | CCCTAACCCTAA--iAmMC6T-TTAGGGTTAG | 50 |
| ID No. | DP-1 | GGCAGCTGAGGTGTCTGATAATACTAAT | |
| 124 | | | |
| Sequence | HPV 27 | CCCTAACCCTAA--iAmMC6T-TTAGGGTTAG | 55 |
| ID No. | DP-2 | GGGTGTGCAGCTGAGGTGTCTGATAATACT | |
| 125 | | AAT | |
| Sequence | HPV 30 | CCCTAACCCTAA--iAmMC6T-TTAGGGTTAG | 44 |
| ID No. | DP-1 | GGAACCACACAAACGTTATCCA | |
| 126 | | | |
| Sequence | HPV 30 | CCCTAACCCTAA--iAmMC6T-TTAGGGTTAG | 50 |
| ID No. | DP-2 | GGATCTGCAACCACACAAACGTTATCCA | |
| 127 | | | |
| Sequence | HPV 31 | CCCTAACCCTAA--iAmMC6T-TTAGGGTTAG | 46 |
| ID No. | DP-1 | GGCTGCAATTGCAAACAGTGATAC | |
| 128 | | | |
| Sequence | HPV 31 | CCCTAACCCTAA--iAmMC6T-TTAGGGTTAG | 49 |
| ID No. | DP-2 | GGTTTGTGCTGCAATTGCAAACAGTGATAC | |
| 129 | | | |
| Sequence | HPV 32 | CCCTAACCCTAA--iAmMC6T-TTAGGGTTAG | 49 |
| ID No. | DP-1 | GGGACACATACAAGTCTACTAACTTTA | |
| 130 | | | |
| Sequence | HPV 32 | CCCTAACCCTAA--iAmMC6T-TTAGGGTTAG | 55 |
| ID No. | DP-2 | GGACTGAAGACACATACAAGTCTACTAACT | |
| 131 | | TTA | |
| Sequence | HPV 33 | CCCTAACCCTAA--iAmMC6T-TTAGGGTTAG | 49 |
| ID No. | DP-1 | GGGCACACAAGTAACTAGTGACAGTAC | |
| 132 | | | |
| Sequence | HPV 33 | CCCTAACCCTAA--iAmMC6T-TTAGGGTTAG | 55 |
| ID No. | DP-2 | GGCTTTATGCACACAAGTAACTAGTGACAG | |
| 133 | | TAC | |
| Sequence | HPV 34 | CCCTAACCCTAA--iAmMC6T-TTAGGGTTAG | 43 |
| ID No. | DP-1 | GGCCACAAGTACAACTGCACC | |
| 134 | | | |
| Sequence | HPV 34 | CCCTAACCCTAA--iAmMC6T-TTAGGGTTAG | 47 |
| ID No. | DP-2 | GGCAATCCACAAGTACAACTGCACC | |
| 135 | | | |
| Sequence | HPV 35 | CCCTAACCCTAA--iAmMC6T-TTAGGGTTAG | 49 |
| ID No. | DP-1 | GGTCTGCTGTGTCTTCTAGTGACAGTA | |
| 136 | | | |
| Sequence | HPV 35 | CCCTAACCCTAA--iAmMC6T-TTAGGGTTAG | 54 |
| ID No. | DP-2 | GGTGTGTTCTGCTGTGTCTTCTAGTGACAGT | |
| 137 | | A | |
| Sequence | HPV 39 | CCCTAACCCTAA--iAmMC6T-TTAGGGTTAG | 53 |
| ID No. | DP-1 | GGACCTCTATAGAGTCTTCCATACCTTCTAC | |
| 138 | | | |
| Sequence | HPV 39 | CCCTAACCCTAA--iAmMC6T-TTAGGGTTAG | 59 |
| ID No. | DP-2 | GGTTATCTACCTCTATAGAGTCTTCCATACC | |
| 139 | | TTCTAC | |
| Sequence | HPV 40 | CCCTAACCCTAA--iAmMC6T-TTAGGGTTAG | 40 |
| ID No. | DP-1 | GGAGTCCCCCACACCAAC | |
| 140. | | | |
| Sequence | HPV 40 | CCCTAACCCTAA--iAmMC6T-TTAGGGTTAG | 46 |
| ID No. | DP-2 | GGCCACACAGTCCCCCACACCAAC | |
| 141 | | | |
| Sequence | HPV 42 | CCCTAACCCTAA--iAmMC6T-TTAGGGTTAG | 42 |
| ID No. | DP-1 | GGCACTGCAACATCTGGTGA | |
| 142 | | | |
| Sequence | HPV 42 | CCCTAACCCTAA--iAmMC6T-TTAGGGTTAG | 48 |
| ID No. | DP-2 | GGGTGTGCCACTGCAACATCTGGTGA | |
| 143 | | | |
| Sequence | HPV 43 | CCCTAACCCTAA--iAmMC6T-TTAGGGTTAG | 46 |
| ID No. | DP-1 | GGGCCCAGTACATATGACAATGCA | |
| 144 | | | |
| Sequence | HPV 43 | CCCTAACCCTAA--iAmMC6T-TTAGGGTTAG | 52 |
| ID No. | DP-2 | GGTACTGTGCCCAGTACATATGACAATGCA | |
| 145 | | | |
| Sequence | HPV 44 | CCCTAACCCTAA--iAmMC6T-TTAGGGTTAG | 42 |
| ID No. | DP-1 | GGTACACAGTCCCCTCCGTC | |
| 146 | | | |
| Sequence | HPV 44 | CCCTAACCCTAA--iAmMC6T-TTAGGGTTAG | 48 |
| ID No. | DP-2 | GGTGCCACTACACAGTCCCCTCCGTC | |
| 147 | | | |
| Sequence | HPV 45 | CCCTAACCCTAA--iAmMC6T-TTAGGGTTAG | 43 |
| ID No. | DP-1 | GGCACAAAATCCTGTGCCAAG | |
| 148 | | | |
| Sequence | HPV 45 | CCCTAACCCTAA--iAmMC6T-TTAGGGTTAG | 49 |
| ID No. | DP-2 | GGCCTCTACACAAAATCCTGTGCCAAG | |
| 149 | | | |
| Sequence | HPV 51 | CCCTAACCCTAA--iAmMC6T-TTAGGGTTAG | 44 |
| ID No. | DP-1 | GGGGTTTCCCCAACATTTACTC | |
| 150 | | | |
| Sequence | HPV 51 | CCCTAACCCTAA--iAmMC6T-TTAGGGTTAG | 47 |
| ID No. | DP-2 | GGTGCGGTTTCCCCAACATTTACTC | |
| 151 | | | |
| Sequence | HPV 52 | CCCTAACCCTAA--iAmMC6T-TTAGGGTTAG | 46 |
| ID No. | DP-1 | GGGCTGAGGTTAAAAAGGAAAGCA | |
| 152 | | | |
| Sequence | HPV 52 | CCCTAACCCTAA--iAmMC6T-TTAGGGTTAG | 54 |
| ID No. | DP-2 | GGCTTTATGTGCTGAGGTTAAAAAGGAAAG | |
| 153 | | CA | |
| Sequence | HPV 53 | CCCTAACCCTAA--iAmMC6T-TTAGGGTTAG | 47 |
| ID No. | DP-1 | GGCGCAACCACACAGTCTATGTCTA | |
| 154 | | | |
| Sequence | HPV 53 | CCCTAACCCTAA--iAmMC6T-TTAGGGTTAG | 54 |
| ID No. | DP-2 | GGCTCTTTCCGCAACCACACAGTCTATGTC | |
| 155 | | TA | |
| Sequence | HPV 54 | CCCTAACCCTAA--iAmMC6T-TTAGGGTTAG | 41 |
| ID No. | DP-1 | GGTACAGCATCCACGCAGG | |
| 156 | | | |
| Sequence | HPV 54 | CCCTAACCCTAA--iAmMC6T-TTAGGGTTAG | 47 |
| ID No. | DP-2 | GGGTGTGCTACAGCATCCACGCAGG | |
| 157 | | | |
| Sequence | HPV 55 | CCCTAACCCTAA--iAmMC6T-TTAGGGTTAG | 48 |
| ID No. | DP-1 | GGCTACAACTCAGTCTCCATCTACAA | |
| 158 | | | |
| Sequence | HPV 55 | CCCTAACCCTAA--iAmMC6T-TTAGGGTTAG | 54 |
| ID No. | DP-2 | GGGTGCTGCTACAACTCAGTCTCCATCTAC | |
| 159 | | AA | |
| Sequence | HPV 56 | CCCTAACCCTAA--iAmMC6T-TTAGGGTTAG | 58 |
| ID No. | DP-1 | GGGACTATTAGTACTGCTACAGAACAGTTA | |
| 160 | | AGTAAA | |
| Sequence | HPV 56 | CCCTAACCCTAA--iAmMC6T-TTAGGGTTAG | 49 |
| ID No. | DP-2 | GGTACTGCTACAGAACAGTTAAGTAAA | |
| 161 | | | |
| Sequence | HPV 57 | CCCTAACCCTAA--iAmMC6T-TTAGGGTTAG | 48 |
| ID No. | DP-1 | GGCCACTGTAACCACAGAAACTAATT | |
| 162 | | | |
| Sequence | HPV 57 | CCCTAACCCTAA--iAmMC6T-TTAGGGTTAG | 53 |
| ID No. | DP-2 | GGGTGTGCCACTGTAACCACAGAAACTAAT | |
| 163 | | T | |
| Sequence | HPV 58 | CCCTAACCCTAA--iAmMC6T-TTAGGGTTAG | 47 |
| ID No. | DP-1 | GGTGCACTGAAGTAACTAAGGAAGG | |
| 164 | | | |
| Sequence | HPV 58 | CCCTAACCCTAA--iAmMC6T-TTAGGGTTAG | 54 |
| ID No. | DP-2 | GGGACATTATGCACTGAAGTAACTAAGGA | |
| 165 | | AGG | |
| Sequence | HPV 59 | CCCTAACCCTAA--iAmMC6T-TTAGGGTTAG | 53 |
| ID No. | DP-1 | GGTCTATTCCTAATGTATACACACCTACCA | |
| 166 | | G | |
| Sequence | HPV 59 | CCCTAACCCTAA--iAmMC6T-TTAGGGTTAG | 58 |
| ID No. | DP-2 | GGCTTCTTCTATTCCTAATGTATACACACCT | |
| 167 | | ACCAG | |
| Sequence | HPV 61 | CCCTAACCCTAA--iAmMC6T-TTAGGGTTAG | 44 |
| ID No. | DP-1 | GGTGCTACATCCCCCCCTGTAT | |
| 168 | | | |
| Sequence | HPV 61 | CCCTAACCCTAA--iAmMC6T-TTAGGGTTAG | 51 |
| ID No. | DP-2 | GGTTTGTACTGCTACATCCCCCCCTGTAT | |
| 169 | | | |
| Sequence | HPV 62 | CCCTAACCCTAA--iAmMC6T-TTAGGGTTAG | 44 |
| ID No. | DP-1 | GGACTATTTGTACCGCCTCCAC | |
| 170 | | | |
| Sequence | HPV 62 | CCCTAACCCTAA--iAmMC6T-TTAGGGTTAG | 49 |
| ID No. | DP-2 | GGACTATTTGTACCGCCTCCACTGCTG | |
| 171 | | | |
| Sequence | HPV 66 | CCCTAACCCTAA--iAmMC6T-TTAGGGTTAG | 50 |
| ID No. | DP-1 | GGAATGCAGCTAAAAGCACATTAACTAA | |
| 172 | | | |
| Sequence | HPV 66 | CCCTAACCCTAA--iAmMC6T-TTAGGGTTAG | 55 |
| ID No. | DP-2 | GGCTATTAATGCAGCTAAAAGCACATTAAC | |
| 173 | | TAA | |
| Sequence | HPV 67 | CCCTAACCCTAA--iAmMC6T-TTAGGGTTAG | 47 |
| ID No. | DP-1 | GGAAAATCAGAGGCTACATACAAAA | |
| 174 | | | |
| Sequence | HPV 67 | CCCTAACCCTAA--iAmMC6T-TTAGGGTTAG | 54 |
| ID No. | DP-2 | GGCTGAGGAAAAATCAGAGGCTACATACA | |
| 175 | | AAA | |
| Sequence | HPV 68b | CCCTAACCCTAA--iAmMC6T-TTAGGGTTAG | 55 |
| ID No. | DP-1 | GGCTACTACTACTGAATCAGCTGTACCAAA | |
| 176 | | TAT | |
| Sequence | HPV 68b | CCCTAACCCTAA--iAmMC6T-TTAGGGTTAG | 60 |
| ID No. | DP-2 | GGTTTGTCTACTACTACTGAATCAGCTGTA | |
| 177 | | CCAAATAT | |
| Sequence | HPV | CCCTAACCCTAA--iAmMC6T-TTAGGGTTAG | 47 |
| ID No. | 68aDP-1 | GGCAGACTCTACTGTACCAGCTG | |
| 178 | | | |
| Sequence | HPV | CCCTAACCCTAA--iAmMC6T-TTAGGGTTAG | 47 |
| ID No. | 68aDP-2 | GGTACAGACTCTACTGTACCAGCTG | |
| 179 | | | |
| Sequence | HPV | CCCTAACCCTAA--iAmMC6T-TTAGGGTTAG | 50 |
| ID No. | 68aDP-3 | GGTACTACAGACTCTACTGTACCAGCTG | |
| 180 | | | |
| Sequence | HPV | CCCTAACCCTAA--iAmMC6T-TTAGGGTTAG | 47 |
| ID No. | 68aDP-4 | GGCAGACTCTACTGTACCAGCTGTG | |
| 181 | | | |
| Sequence | HPV 69 | CCCTAACCCTAA--iAmMC6T-TTAGGGTTAG | 49 |
| ID No. | DP-1 | GGCACAATCTGCATCTGCCACTTTTAA | |
| 182 | | | |
| Sequence | HPV 69 | CCCTAACCCTAA--iAmMC6T-TTAGGGTTAG | 56 |
| ID No. | DP-2 | GGGTATCTGCACAATCTGCATCTGCCACTT | |
| 183 | | TTAA | |
| Sequence | HPV 70 | CCCTAACCCTAA--iAmMC6T-TTAGGGTTAG | 41 |
| ID No. | DP-1 | GGCCGAAACGGCCATACCT | |
| 184 | | | |
| Sequence | HPV 70 | CCCTAACCCTAA--iAmMC6T-TTAGGGTTAG | 46 |
| ID No. | DP-2 | GGCTGCACCGAAACGGCCATACCT | |
| 185 | | | |
| Sequence | HPV 72 | CCCTAACCCTAA--iAmMC6T-TTAGGGTTAG | 45 |
| ID No. | DP-1 | GGCACAGCGTCCTCTGTATCAGA | |
| 186 | | | |
| Sequence | HPV 72 | CCCTAACCCTAA--iAmMC6T-TTAGGGTTAG | 51 |
| ID No. | DP-2 | GGTACTGCCACAGCGTCCTCTGTATCAGA | |
| 187 | | | |
| Sequence | HPV 73 | CCCTAACCCTAA--iAmMC6T-TTAGGGTTAG | 51 |
| ID No. | DP-1 | GGAGGTACACAGGCTAGTAGCTCTACTAC | |
| 188 | | | |
| Sequence | HPV 73 | CCCTAACCCTAA--iAmMC6T-TTAGGGTTAG | 54 |
| ID No. | DP-2 | GGTGTAGGTACACAGGCTAGTAGCTCTACT | |
| 189 | | AC | |
| Sequence | HPV 81 | CCCTAACCCTAA--iAmMC6T-TTAGGGTTAG | 44 |
| ID No. | DP-1 | GGGCTACATCTGCTGCTGCAGA | |
| 190 | | | |
| Sequence | HPV 81 | CCCTAACCCTAA--iAmMC6T-TTAGGGTTAG | 52 |
| ID No. | DP-2 | GGTTTGCACAGCTACATCTGCTGCTGCAGA | |
| 191 | | | |
| Sequence | HPV 82 | CCCTAACCCTAA--iAmMC6T-TTAGGGTTAG | 43 |
| ID No. | DP-1 | GGCTCCAGCAAACTTTAAGCA | |
| 192 | | | |
| Sequence | HPV 82 | CCCTAACCCTAA--iAmMC6T-TTAGGGTTAG | 48 |
| ID No. | DP-2 | GGCTCCAGCAAACTTTAAGCAATACA | |
| 193 | | | |
| Sequence | HPV 83 | CCCTAACCCTAA--iAmMC6T-TTAGGGTTAG | 51 |
| ID No. | DP-1 | GGTGCTGCTACACAGGCTAATGA | |
| 194 | | | |
| Sequence | HPV 83 | CCCTAACCCTAA--iAmMC6T-TTAGGGTTAG | 50 |
| ID No. | DP-2 | GGTCAGCTGCTGCTACACAGGCTAATGA | |
| 195 | | | |
| Sequence | HPV 84 | CCCTAACCCTAA--iAmMC6T-TTAGGGTTAG | 48 |
| ID No. | DP-1 | GGACCGAATCAGAATATAAACCTACCAAT | |
| 196 | | | |
| Sequence | HPV 84 | CCCTAACCCTAA--iAmMC6T-TTAGGGTTAG | 55 |
| ID No. | DP-2 | GGCAACACCGAATCAGAATATAAACCTACC | |
| 197 | | AAT | |
| Sequence | HPV 90 | CCCTAACCCTAA--iAmMC6T-TTAGGGTTAG | 47 |
| ID No. | DP-1 | GGACAAACACCCTCTGACACATACA | |
| 198 | | | |
| Sequence | HPV 90 | CCCTAACCCTAA--iAmMC6T-TTAGGGTTAG | 51 |
| ID No. | DP-2 | GGCCACACAAACACCCTCTGACACATACA | |
| 199 | | | |
| Sequence | HPV 91 | CCCTAACCCTAA--iAmMC6T-TTAGGGTTAG | 52 |
| ID No. | DP-1 | GGTCTGTGCTACCTACTACATATGACAACA | |
| 200 | | | |
| Sequence | HPV 91 | CCCTAACCCTAA--iAmMC6T-TTAGGGTTAG | 58 |
| ID No. | DP-2 | GGACTGAGTCTGTGCTACCTACTACATATG | |
| 201 | | ACAACA | |
| Sequence | HPV U | CCCTAACCCTAA--iAmMC6T-TTAGGGTTAG | 54 |
| ID No. | DP-1 | GGTTGTTGGGDTAATCAGTTGTTTGTTACTG | |
| 202 | | T | |
| Sequence | HPV U | CCCTAACCCTAA--iAmMC6T-TTAGGGTTAG | 56 |
| ID No. | DP-2 | GGTTTGTTACTGTTGTAGATACTACTCGCA | |
| 203 | | GTAC | |
| Sequence | HPV U | CCCTAACCCTAA--iAmMC6T-TTAGGGTTAG | 54 |
| ID No. | DP-3 | GGTTGTTGGGDTAATCARTTRTTTGTTACDG | |
| 204 | | T | |
| Sequence | HPV U | CCCTAACCCTAA--iAmMC6T-TTAGGGTTAG | 47 |
| ID No. | DP-4 | GGTTTKTTACHGTKGTDGATACYAC | |
| 205 | | | |
| Sequence | HPV U | CCCTAACCCTAA--iAmMC6T-TTAGGGTTAG | 49 |
| ID No. | DP-5 | GGTATTTRTTACTGTTGTDGAYACYAC | |
| 206 | | | |
| Sequence | HPV U | CCCTAACCCTAA--iAmMC6T-TTAGGGTTAG | 49 |
| ID No. | DP-6 | GGTATTTGTAACTGTTGTGGATACCAC | |
| 207 | | | |
| Sequence | HPV U | CCCTAACCCTAA--iAmMC6T-TTAGGGTTAG | 47 |
| ID No. | DP-7 | GGTTTRTTACTGTTGTDGAYACYAC | |
| 208 | | | |
| Sequence | HPV U | CCCTAACCCTAA--iAmMC6T-TTAGGGTTAG | 49 |
| ID No. | DP-8 | GGTATTTRTTACTGTTGTDGAYACYAC | |
| 209 | | | |
| Sequence | ACTB-1DP | CCCTAACCCTAA--iAmMC6T-TTAGGGTTAG | 46 |
| ID No. | | GGACCCCGTGCTGCTGACCGAGGC | |
| 210 | | | |
| Sequence | ACTB-2DP | CCCTAACCCTAA--iAmMC6T-TTAGGGTTAG | 43 |
| ID No. | | GGCACCCCGTGCTGCTGACCG | |
| 211 | | | |
| Sequence | ACTB-3DP | CCCTAACCCTAA--iAmMC6T-TTAGGGTTAG | 47 |
| ID No. | | GGCACCCCGTGCTGCTGACCGAGGC | |
| 212 | | | |
| Sequence | ACTB-4DP | CCCTAACCCTAA--iAmMC6T-TTAGGGTTAG | 44 |
| ID No. | | GGGCTGCGTGTGGCTCCCGAGG | |
| 213 | | | |

(In the sequences, n means iAmMC6T.)

### EXAMPLE 7

### Fabrication of DNA Chip

Grid was designed corresponding to the probes designed in Example 6 and the probes mixed with a suitable buffer were spotted on a glass slide for a microscope. Then, the slide was stabilized with suitable treatment and stored until test after quality control. Details are as follows.

1. Preparation of Grid to be Position on DNA Chip

A grid was prepared so as to determine quickly and easily whether the HPV detected on the chip is high-risk type, moderate-risk type or low-risk type as shown in FIG. 1. As seen from FIG. 1, 14 probes for high-risk type HPV were spotted on the left two lines and probes for moderate-risk type HPV L1 were spotted on the bottom of the second line. 14 probes for low-risk type HPV were spotted on the third line and 8 probes for other type and a universal L1 probe were spotted on the rightmost line. For HPV-68, a 1:1 mixture of HPV-68a and 68b probes was spotted. Also, a total of 12 oligonucleotide probes specific for human beta-actin gene were spotted on the 11×11 grid between each L1 probe to serve as corner markers and confirm suitability of DNA isolation and PCR amplification for quality control (QC).

In addition to the human beta-actin gene, globin or glyceraldehyde-3-phosphate dehydrogenase gene may be used as standard marker probe.

Each oligonucleotide probe was spotted using an arrayer. The same probes were spotted in duplicate in order that each genotype of HPV is detected at least twice.

2. Preparation of Solution for Spotting Oligonucleotide Probes on Chip and Transfer to Master Plate

Probes synthesized by attaching 5′-C6 amine in Example 6 were purified by high-performance liquid chromatography (HPLC) and dissolved in sterilized triply distilled water to a final concentration of 200 pM. Thus prepared probes were mixed with 4.3 times the volume of a microspotting solution to make the final concentration 38 pM. The resulting mixtures were sequentially transferred to a 384-well master plate.

3. Spotting and Fixation of Probes

Q arrayer2 (Genetixs, UK) or an arrayer comparable thereto was used to transfer the spotting solution containing the probes from the master plate to an aldehyde-coated glass slide and spot each probe in duplicate (double hit). The glass slide may be Luminano Aldehyde LSAL-A, a silicon wafer or a product comparable thereto. Each spot can be 10-200 μm in size. The DNA chip fabricated by spotting the probes onto the glass slide was reacted at room temperature for 15 minutes in a glass jar maintained at 80% humidity and then post-treated according to a known method (Zammatteo, N., L. Jeanmart, S. Hamels, S. Courtois, P. Louette, L. Hevesi, and J. Remacle. 2000. Comparison between different strategies of covalent attachment of DNA to glass surfaces to build DNA microarrays. Anal. Biochem. 280: 143-150.).

4. Washing and Storage of Microarray

A. Preparation of Reagent

1) 10% sodium dodecyl sulfate (SDS; 100 mL): 10 g of SDS (Sigma, L4509-1KG) reagent is weighed into a 500-mL beaker. After adding distilled water (ultrapure water) to make a final volume 100 mL and dissolving, the solution is kept in a sealed container at room temperature.

2) 0.1% SDS (4 L): 10 mL of 10% SDS is added to four respective 1-L containers. After adding distilled water (ultrapure water) to make a final volume 1 L and mixing, the solutions are kept in a sealed container at room temperature.

3) 1 M ethanolamine solution (300 mL): 18.3 mL of 16.6 M ethanolamine solution (Sigma, E0135) is added to a 500-mL container. After adding distilled water (ultrapure water) to make a final volume 300 mL and mixing, the solution is kept in a sealed container at room temperature. Light is blocked since the solution sensitive to light.

4) Blocking solution (425 mL): A blocking solution is prepared immediately before use. 1× PBS (300 mL) is mixed with 100% ethanol (100 mL) and 1 M ethanolamine (25 mL).

5) 1× phosphate buffer: Five PBS buffer tablets (Sigma, P4417) are dissolved by adding 0.9 L of distilled water (ultrapure water). After adjusting pH to 7.4 with 10 N HCl, the final volume is adjusted to 1 L.

6) 25% ethanol solution: 250 mL of 100% ethanol (Merck, 1.00983.2511) is added to 1-L container. After adding distilled water (ultrapure water) to make a final volume 1 L, the solution is kept in a sealed container at room temperature.

B. Washing of Microarray

1) A reactor, a washing container and reagents (0.1% SDS, 1 M ethanolamine, 1× phosphate buffer, 100% ethanol and 25% ethanol) are prepared.

2) 300 mL of 0.1% SDS solution is added to the washing container and the slide is washed for 2 minutes at 150 rpm using a reciprocating shaker. This procedure is repeated twice.

3) The slide is washed for 2 minutes at 150 rpm with triply distilled water using a reciprocating shaker. This procedure is repeated twice.

4) Electrically preheated distilled water is added to a washing container dedicated for distilled water and the chip is kept in the water for 3 minutes.

5) The chip is kept in triply distilled water at room temperature for 1 minute.

6) A blocking solution is prepared immediately before use.

7) The chip is kept in the blocking solution for 30 minutes.

8) 300 mL of 25% ethanol solution is added to a washing container and the slide is washed for 2 minutes at 150 rpm using a reciprocating shaker. This procedure is carried out only once.

9) The slide is washed for 2 minutes at 150 rpm with triply distilled water using a reciprocating shaker. This procedure is repeated twice.

10) After washing is completed, the chip is slowly lifted from the last washing solution (water).

11) Water is removed by centrifuging at 1,000 rpm for 3 minutes (MF-600, Hanil Science). 12) The slide is put in a slide box and stored in a desiccator until use.

The DNA chip of the present disclosure fabricated above was used to perform hybridization as described in Example 8.

### EXAMPLE 8

### Hybridization on DNA Chip and Establishment of Analysis Condition

100 artificial standard samples obtained from various combinations of one, two or three clones for each type of HPV in Example 5 were used as templates for PCR amplification of HPV L1 and beta-actin genes. The PCR products were placed on the chip prepared in Examples 6-7 and hybridization was performed at least 3 times. Then, the optimal condition was established by analyzing with a fluorescence scanner Details are as follows.

1. Duplex PCR

PCR of HPV L1 and human beta-actin genes was performed as in Example 3. For a reverse primer among the combination of primers, i.e. GP6−1, GP6+ and ACTBR, Cy-5-labeled oligonucleotide was used.

The label may be replaced by Cy3, Cy5, Cy5.5, BODIPY, Alexa 488, Alexa 532, Alexa 546, Alexa 568, Alexa 594, Alexa 660, rhodamine, TAMRA, FAM, FITC, Fluor X, ROX, Texas Red, Orange Green 488X, Orange Green 514X, HEX, TET, JOE, Oyster 556, Oyster 645, BODIPY 630/650, BODIPY 650/665, Calfluor Orange 546, Calfluor Red 610, Quasar 670, biotin or AuNP (gold nanoparticle having a diameter of 5 nm, 10 nm, 20 nm or 50 nm). Also, silver core shell or silver enhancement may be used. In particular, when AuNP or silver core shell is used as the label, a target probe having a thiol group at 3′-terminal and thus capable of complementarily binding to the PCR template is attached for hybridization with the gold nanoparticle and silver enhancement is carried out or a silver shell is formed on the target probe bound to the gold nanoparticle. After the reaction, reflectivity of the chip is measured using a PD scanner, not a general fluorescence scanner using PMT as a detector, or SEM images are taken for detection.

2. Hybridization Reaction

Hybridization reaction is carried out after placing the HPV PCR products amplified by PCR on a slide substrate on which various HPV oligonucleotide probes are immobilized. A 100-μL 8-well perfusion chamber (Schleicher & Schuell BioScience, Germany) is used as a hybridization chamber. Details are as follows.

1) Fresh 1.5-mL or 200-μL tubes are prepared corresponding to the number of samples.

2) 50 μL of purified water is added to each tube.

3) 15 μL of the duplex PCR products of L1 and ACTB genes are added and mixed well.

4) The tube is allowed to stand on a heat block maintained at 95° C. for 3 minutes.

5) The tube is then allowed to stand on ice for 5 minutes.

6) The reaction tube is centrifuged for 30 seconds.

7) 65 μL of HYB I solution (2 mL of 20×SSC, 6.3 mL of 5× phosphate buffer and 1.7 mL of 90% glycerol, final volume: 10 mL) is added to the tube and mixed well with a pipette.

8) The prepared reaction solution is slowly injected into the injection port on the coverslip attached to the chip surface. It is checked whether foams are observed between the chip and the well cover. If any, the foams are removed by sweeping with a gloved hand.

9) The chip is subjected to hybridization in a reaction bath at 48° C. for 30 minutes.

3. Washing

1) After the hybridization is completed, the well cover is removed from the chip.

2) Previously prepared washing solution 1 is added to a washing container such that the chip is immersed and the chip is washed at room temperature for 2 minutes with a speed of 8 oscillations using a reciprocating shaker. If the number of the chip is one, it may be washed in a 50-mL conical tube holding 40 mL of washing solution by shaking the tube up and down for 2 minutes at a speed of 50 reciprocations per minute. When the washing is carried out manually without using the reciprocating shaker, washing solution is added to a washing container such that the chip is immersed and the washing container is shaken left and right for 2 minutes at a speed of 50 reciprocations per minute.

3) The used washing solution is discarded and fresh washing solution 1 is added. Washing is performed again for 2 minutes.

4) The used washing solution is discarded and fresh washing solution 1 is added. Washing is performed again for 2 minutes.

5) The used washing solution is discarded and fresh washing solution 2 is added. Washing is performed again for 2 minutes.

6) After the washing, a spin dryer or an air compressor may be used to remove the buffer remaining on the chip.

4. Scanning

After the hybridization followed by removal of non-specific signals through washing, the dried slide was scanned with a scanner to analyze chip images. As for the scanner, Genepix 4000B, Easy Scan-1, Affymetrix 428 Array Scanner (Affymetrix, USA), ScanArray Lite (Packard Bioscience, USA) or an instrument comparable thereto may be used.

### EXAMPLE 9

### Analysis of Cervical Clinical Samples on DNA Chip

Duplex PCR was carried out again as described in Example 3 on the DNA of cervical clinical samples of which the presence or absence of HPV and type thereof were identified by post-PCR sequencing in Examples 3-4. The PCR products were placed on the DNA chip fabricated in Examples 6-7 and hybridization was carried out as in Example 8. After washing, analysis was carried out using a fluorescence scanner. Sensitivity, specificity and reproducibility of the DNA chip were analyzed and the optimal condition of the DNA chip of the present disclosure for genotyping of HPV was evaluated again. The results are shown in FIGS. 5-13.

FIGS. 5-13 show the result of carrying out hybridization reactions for samples infected with various types of HPV using 45 oligonucleotide probes spotted on the DNA chip of the present disclosure. As seen from the figures, hybridization occurred type-specifically for each probe without cross-hybridization.

That is to say, the 45 probes specific for the HPV types of the DNA chip bound specifically to the DNA of the respective types of HPV without cross-hybridization between the probes. In addition, the samples coinfected by more than one type of HPV could be accurately diagnosed. That is to say, the DNA chip of the present disclosure exhibited 100% sensitivity and 100% specificity for diagnosis of single and mixed infection by HPV. Further, 100% reproducibility was exhibited as the same results were obtained when different testers carried out the diagnosis three or more times with time intervals. The 45 probes synthesized according to the present disclosure could accurately analyze a large number of combinations of HPV types which could not be handled with the existing DNA microarrays.

In particular, FIG. 14 is a scanning image showing a result of extracting DNA from a cervical swab sample of a Korean woman who had high grade squamous intraepithelial lesion histologically identified in the cervix, performing duplex PCR according to the present disclosure and performing hybridization of the HPV L1 and beta-actin amplification products on the HPV DNA chip of the present disclosure.

The DNA chip fabricated according to the present disclosure could accurately diagnose the type of HPV from the cervical swab samples. The probe for each HPV type bound specifically to the DNA of specific type of HPV and no cross-hybridization occurred between the probes. In addition, even the samples coinfected by more than one type of HPV, which are difficult to diagnose through direct sequencing and can be diagnosed by many sequencing assays after cloning, could be accurately diagnosed with the DNA chip of the present disclosure. That is to say, the DNA chip of the present disclosure exhibited 100% sensitivity and 100% specificity for diagnosis of single and mixed infection by HPV. Further, 100% reproducibility was exhibited as the same results were obtained when different testers carried out the diagnosis three or more times with time intervals.

### EXAMPLE 10

### Correlation of Diagnosis of Cervical Clinical Sample Using DNA Chip with Clinical Data

The result of analysis using the DNA chip after PCR in Example 9 was compared with clinical data obtained by cervical tissue testing, Pap smear, etc. in order to analyze their correlation and investigate whether the DNA chip of the present disclosure is useful for predicting cervical cancer or precancerous lesions. It was demonstrated that the DNA chip of the present disclosure is useful not only for genotyping of HPV but also for screening of cervical cancer.

Among the 15,708 cervical cell samples from Korean women, HPV infection was identified in 7,371 samples. The prevalence rate was 463.93%. 45 types of HPV were identified. Among the detected HPV types, HPV-16 was the most common, followed by HPV-53, HPV-39, HPV-56, HPV-58, HPV-52, HPV-70, HPV-84, HPV-18, HPV-68 and HPV-35. This result is distinguished from that of Europe where HPV-16 is the most common, followed by HPV-18, HPV-45, HPV-52, HPV-31, HPV-33 and HPV-58 (Murinoz N et al., N Engl J Med, 2003, 348: 518-27).

HPV-53 showed high prevalence rate in Koreans but not in Europeans. Accordingly, it can be seen that HPV-53 is the major cause of cervical cancer in Koreans.

### EXAMPLE 11

### Diagnosis of Cervical Samples Using the DNA Chip of the Present Disclosure

The HPV DNA chip of the present disclosure was used for diagnosis of cervical samples. The purposes of the test were, first, to investigate how accurately the HPV DNA chip can diagnose HPV infection and the genotype of HPV and, second, to evaluate how helpful it is in predicting cancers and important cervical lesions including precancerous lesions. For this, DNA was isolated from cervical swab samples of Korean women who were suspected of cervical HPV infection and lesions and subjected to (1) test with the HPV DNA microarray of the present disclosure, (2) PCR of the HPV L1 gene followed by automated sequencing analysis, and (3) test by Hybrid Capture Assay-II (HCA-II; Digene Corporation) which is an HPV DNA test approved by the USFDA.

The HPV DNA chip of the present disclosure enables detection of all the 43 HPV types invading human cervix, anus, oral cavity, etc., whereas HCA-II tests 12 high-risk type HPVs. Comparison was made while focusing on (1) the sensitivity and specificity of diagnosis of HPV infection, (2) the accuracy of HPV genotype diagnosis, and (3) the accuracy of prediction of cervical cancer and serious lesions including precancerous lesions. The HPV DNA microarray test was carried out as described in Examples 2 and 8 and PCR and base sequencing were performed according to the known method (Kim K H, Yoon M S, Na Y J, Park C S, Oh M R, Moon W C. Development and evaluation of a highly sensitive human papillomavirus genotyping DNA chip. Gynecol Oncol. 2006; 100(1): 38-43). HCA-II test was performed according to the manufacturer's instructions.

The 201 subjects tested were aged between 18 and 81, and the average age was 52.4 years. The result of performing PCR of the HPV L1 gene is summarized in Table 7. HPV infection was identified from 191 subjects out of the 201 subjects. 149 cases were high-risk HPV and 72 cases were mixed infections by more than one HPV type.

The analysis result with the HPV DNA chip of the present disclosure was compared with that of HCA-II (Tables 7-10). The HPV DNA chip of the present disclosure accurately diagnosed all (100%) the 191 cases of positive HPV infection. Among them, 174 cases (91.1%) were accurately genotyped. Although the 149 high-risk cases were accurately identified, rare types of HPV could not be identified with the chip of the present disclosure. Meanwhile, HCA-II failed to detect 40 cases of HPV from the 191 cases of HPV-positive samples and failed to detect 12 cases (8.1%) from among the 149 high-risk HPV infected samples. The HPV DNA chip of the present disclosure could accurately predict all the high-risk type cervical lesions including cervical cancer, cervical intraepithelial neoplasia (CIN) and high-grade squamous intraepithelial lesion (HSIL). In contrast, the HCA-II test failed to detect one of the 8 cases of cervical cancer and one of the 12 cases of HSIL. In addition, the HPV chip of the present disclosure showed better ability of detecting low-grade SIL than HCA-II (92.2% vs. 56.9%, p<0.05).

These results reveal that the HPV DNA chip of the present disclosure exhibits nearly 100% sensitivity in diagnosis of HPV infection and genotyping of HPV, especially high-risk HPV, and is excellent in predicting cervical cancer and precancerous lesions. Further, it is superior to the existing HCA-II test.

TABLE 7
Result of HPV genotyping using the DNA chip of the present disclosure

| [symbol] | No Cases (%) |
|---|---|
| Total | 201 |
| Positive for HPV | 191 |
| Single infection | 119 |
| Mixed infection | 72 |
| High risk HPV | 149 (74.9) |
| Low risk HPV | 48 |
| Undetermined risk | 31 |
| Rare type | 17 |
| Individual type | |

TABLE 8
Comparison of the HPV DNA chip of the present disclosure with Hybrid
Capture Assay (HCA)-II

| | Accuracy (%) | |
|---|---|---|
| | HPV DNA Chip | HCA-II |
| Detection of HPV | 191/191 (100.0) | 151/191 (79.1) |
| Detection of high risk HPV | 149/149 (100.0) | 137/149 (91.9) |
| Genotyping of HPV | 174/191 (91.1)* | Not analyzable |

*The 17 types are not included in the HPV DNA chip.

TABLE 9
Analysis of the cases where detection was failed with HCA-II

| | |
|---|---|
| Total | 33/171 (19.3%) |
| High risk | 12*/149 (8.1%) |
| Probable high risk | 5/20 (25.0%) |
| Low risk** | 48/48 (100.0%) |

*The types are 16, 33, 35 and 68 which are all included in HCA-II.
**These types are not included in HCA-II.

TABLE 10
Comparison of the HPV DNA chip of the present disclosure with HCA-II
for diagnosis of cervical cancer and precancerous lesions

| | Sensitivity (%) | |
|---|---|---|
| Cytopathological diagnosis | HPV DNA Chip | HCA-II |
| Carcinoma | 8/8 (100.0) | 7/8 (87.5) |
| CIN, grade 3/3 | 1/1 | 1/1 |
| High grade SIL | 12/12 (100.0) | 11/12 (91.7) |
| Low grade SIL | 94/102 (92.2)* | 58/102 (56.9) |
| Carcinoma + CIN + HSIL | 20/20 (100.0) | 19/20 (95.0) |
| All | 115/123 (93.5)* | 77/123 (62.6) |

*Significantly different (p < 0.05)

### EXAMPLE 12

### Analysis of Anal and Head and Neck Samples Using HPV DNA Chip

HPV can cause cancer not only in the genitalia but also other in organs and tissues. Indeed, a number of oral cancer, pharyngeal cancer, laryngeal cancer and anal cancer are caused by HPV. Accordingly, the HPV DNA chip of the present disclosure was used to analyze HPV infection in cancer and precancerous lesions. For the experiment, 24 tonsil tissue samples and 179 hemorrhoidal tissue samples obtained from Koreans were tested using the chip of the present disclosure.

Among the 24 tonsil tissue samples, 13 were HPV-positive and 19 were HPV-negative. Of the 13 HPV-positive samples, 5 were single infection and 8 were mixed infection. All the 13 HPV-positive samples were infected by high-risk type HPV (HPV-16: 26%, HPV-56: 13%, HPV-33: 13%, HPV-52: 8%).

The 179 hemorrhoidal tissue samples were acquired from Seoul National University Hospital and Asan Medical Center (19 from females, 160 from males aged between 27 and 83; average age: 40 years). Test using the DNA chip of the present disclosure revealed that 63 samples were HPV-positive, 10 from females and 53 from males. Of the 63 HPV-positive samples, 44 were single infection and 19 were mixed infection. Among the 63 HPV-positive samples, 49 were infected by high-risk type HPV (single and mixed infection) and 14 were infected by low-risk type HPV (HPV-16: 21%, HPV-18: 21%, HPV-68: 8%).

Accordingly, it was confirmed that the DNA chip of the present disclosure can be used to diagnose not only the HPV infection causing cervical cancer but also the HPV infection causing anal or laryngeal cancer.

### EXAMPLE 13

### Labeling of DNA Chip with Gold Nanoparticle

For hybridization in Example 8, the DNA chip was labeled with gold nanoparticles (AuNP; 20 nm in diameter, BBI) or enhanced with silver shell after PCR. That is to say, a target probe having a thiol group at 3′-terminal and thus capable of complementarily binding to the PCR template is attached for hybridization with the gold nanoparticle and silver enhancement is carried out or a silver shell is formed on the target probe bound to the gold nanoparticle. After the reaction, reflectivity of the chip is measured using a PD scanner, not a general fluorescence scanner using PMT as a detector, or SEM images are taken for detection. Details are as follows.

1. Target Probe Design

A target probe for labeling gold nanoparticles is as follows. If the probes spotted on the chip are in forward direction, the PCR template is usually bound in reverse direction. Thus, a sequence capable of complementarily binding to the PCR template bound to the probes on the chip is designed. That is to say, since the terminal of the PCR template binding to the ACTB probe is usually a reverse primer, the target probe is synthesized to have a sequence complementary to the reverse primer. Because the terminal of the target probe should bind with AuNP (20 nm in diameter), an internal C18 linker and 10 adenine residues were inserted following the complementary base sequence and then a 3′-terminal thiol group was added. Thus designed target probe is shown in Table 11. LTP is the target probe for the PCR product of HPV L1 gene and ATP is the target probe for the PCR product of ACTB gene.

TABLE 11
Target probe sequences

| No | Name | Sequence (5′->3′) | Mer |
|---|---|---|---|
| Sequence | LTP | 5′-GAGGAATATGATTTACAGTTTATT-Internal C₁₈ | 34 |
| ID No 214 | | linker-A₁₀-(CH₂)₃-SH-3′ | |
| Sequence | ATP | 5′-GCCAACCGCGAGAAGATGAC-Internal C₁₈ | 30 |
| ID No. | | linker-A10-(CH2)3-SH-3′ | |
| 215 | | | |

2. Attachment of Gold Nanoparticle to PCR Product

The PCR products bound to the oligonucleotide probes spotted on the chip through hybridization are labeled with AuNP by either of the following two methods (FIG. 15). One is silver enhancement (silver staining) and the other is to label the target probe with AuNP, form a silver shell thereon with the AuNP as seed and then attach the silver shell target probe to the PCR product hybridized with the probes. Details are as follows.

I. Cleavage of Disulfide Group of Thiol-Modified Oligonucleotide

In order to bind gold nanoparticle with the target probe, the thiol group of the target probe should be activated.

1) The oligonucleotide probes described in Table 11 are quick spun and dissolved by mixing well with 1,517 μL of distilled water.

2) 15.4 mg of 0.1 M DTT is dissolved in 1 mL of disulfide cleavage buffer (pH 8.0; 170 nM phosphate buffer, 11.468 g Na₂HPO₄, 0.509 g NaH₂PO₄, 500 mL nanopure water).

3) 100 μL of the 0.1 M DTT solution is added to a 1.5-mL tube, mixed well with 100 μL of dissolved oligonucleotide probes (10 nM) and reacted at room temperature for 2 hours.

4) A NAP-5 column (Sephadex G-25 DNA grade, GE Healthcare, Cat. No. 17-0853-02) is prepared by fixing on a stand.

5) The buffer is discarded and the column is washed by filling with DW using a squeeze bottle. This procedure is repeated 3 times for sufficient washing. Then, the column is capped until use.

6) 200 μL of the reacted oligonucleotide probes are loaded in the NAP-5 column. Caution is taken such that bubbles are not formed in the column. After the solution leaves the column (it takes about 1 minute and 25 seconds), 450 μL of distilled water is added. After the solution leaves the column again (it takes about 1 minute and 28 seconds), four drops are collected in each of seven 1.5-mL tubes while adding 950 μL of DW.

II. Determination of Oligonucleotide Probe Concentration

1) Absorbance of 70 μL of the solutions collected in tubes 1, 2 and 5 is measured at 260 nm using a spectrophotometer.

2) The solutions of tubes 1-5 are mixed in tube 2 and absorbance is measured again.

3) Molar concentration is calculated according to the equation C=A/ε.

4) Oligonucleotide probe concentration and AuNP concentration are calculated from the above equation according to the size of AuNP (e.g. 20 nm or 50 nm).

III. Labeling of Target Probe with AuNP

1) Based on the calculation result, 2 mL of AuNP (20 nm) is added to a 15-mL conical tube. After mixing well with 543 μL of oligonucleotide probes, reaction is carried out for 20 minutes in a shaking incubator set to 25° C.

2) After adding 254.356 μL of 100 mM PBS (Na₂HPO₄ 0.562 g+NaH₂PO₄ 0.125 g+H₂O 50 mL), the mixture is incubated for 20 minutes.

3) After adding 2.797 μL of 10% SDS, the mixture is incubated for 20 minutes.

4) After adding 140.035 μL of 2 M NaCl, the mixture is incubated for 20 minutes. This procedure is repeated once more.

5) After adding 70.0179 μL of 2 M NaCl, the mixture is incubated for 20 minutes. This procedure is repeated once more and then the mixture is incubated overnight.

6) The solution is dispensed into two 1.5-mL tubes (1.5 mL each) and centrifuged at 10,000 rpm for 20 minutes. The resulting pellets are resuspended by adding 1 mL of 0.01% SDS solution in 0.3 M PBS (10 mM PB, 40 mL+2 M NaCl, 6 mL). After centrifugation at 10,000 rpm for 20 minutes, the pellet resulting pellets are resuspended by adding 1 mL of 0.3 M PBS (NaCl, 8.766 g+Na₂HPO₄, 0.562 g, NaH₂PO₄, 0.25 g+DW, 500 mL) twice (2 mL in total).

3. Labeling with Silver Shell (Core Shell) with Gold Nanoparticle as Seed

The silver shell thickness is determined based on the absorbance of the target probe-AuNP measured in the step 2. Then, the total amount of silver (Ag) and the amount of other reagents are determined from the data of Table 12.

TABLE 12
Amount of reagents required for 7 mL of silver shell

| LTP-AuNP | ABS = 0.9017 | X 70 | HTP-AuNP | ABS = 0.90309 | X 70 |
|---|---|---|---|---|---|
| DNA-AuNP | 100 μl | 7 ml | DNA-AuNP | 100 μl | 7 ml |
| 1% PVP | 50 μl | 3.5 ml | 1% PVP | 50 μl | 3.5 ml |
| L-SA(10−1M) | 20 μl | 1.4 ml | L-SA(10−1M) | 20 μl | 1.4 ml |
| AgNO3(10−3M) | 55.7 μl | 3.9 ml | AgNO3(10−3M) | 55.8 μl | 3.9 ml |
| Target thickness | 5 nm | 5 nm | Target | 5 nm | 5 nm |
| | | | thickness | | |

1) After sequentially adding the required amounts of DNA-AuNP, 1% PVP, 10⁻¹ M L-SA and 10⁻³ M AgNO₃ and mixing well, the mixture is incubated overnight while shaking at 150 rpm.

2) The solution is dispensed into a 1.5-mL tube and centrifuged at 8,000 rpm for 20 minutes.

3) The supernatant is removed and 1 mL of 0.3 M PBS is added. After mixing well, centrifugation is carried out again at 10,000 rpm for 20 minutes.

4) After removing the supernatant, 0.3 M PBS is added according to the initial volume of AuNP. If the pellets are not resuspended, the mixture is kept in a water bath at 60° C. and then resuspended.

5) Absorbance of the resuspended DNA-AuNP-core shell is measured (λ=260 nm).

4. Hybridization and Washing

1) AuNP-labeled target probe stored at low temperature is suspended in a water bath of 60° C. 100 μL of the target probe is added on the chip and reacted at room temperature for 4 hours.

2) The chip is washed twice with 0.3 M PBS and then dried.

The result of experiments using the probe of the present disclosure is shown in FIGS. 16-21. FIGS. 16-17 show scanning images of HPV-6-AuNP-Ag enhanced and HPV-6-AuNP-core shell treated chips. The images on the left side show a result of scanning all the 8 wells, and the images on the right side show spots spotted in each well. Unlike the silver staining images of FIG. 16, the spots are clearly seen in FIG. 17.

FIGS. 18-19 show a result of analyzing the spots and background of the HPV-6-AuNP-Ag enhanced and HPV-6-AuNP-core shell treated chips by scanning electron microscopy (SEM). It can be seen that gold nanoparticles are present with high density in the HPV-6 probe spot as compared to the background in both chips.

FIG. 20 shows SEM images of the HPV-6-AuNP-Ag enhanced spots and HPV-6-AuNP-Ag core shell-labeled spots. It can be seen that Ag core shell labeling gives much more stable result than Ag staining. Also, it can be seen that, in case of Ag staining, the staining was non-specific.

FIG. 21 shows a result of scanning a chip wherein a PCR template for HPV-6 and a target probe (LTP) are labeled with AuNP (HPV-6-AuNP), a chip wherein a PCR template for HPV-6 and a target probe (LTP) are labeled with AuNP and then enhanced with silver (HPV-6-AuAg staining) and a chip wherein a PCR template for HPV-6 and a target probe (LTP) are labeled first with Au and then with Ag core shell (HPV-6-AuAg Core shell) at different template concentrations using a scanner equipped with a PD and comparing reflectivity of each spot with SBR. It can be seen that the SBR value is the highest when the template concentration is 1 pmol. In particular, the reflectivity was the best when second labeling was carried out with silver core shell, with HPV-6-AuNP<HPV-6-AuAg staining<HPV-60AuAg core shell. Accordingly, it can be seen that nanoparticle labeling is applicable to the chip of the present disclosure.

As described in the foregoing examples, the HPV DNA chip of the present disclosure is useful for detecting the presence of 43 types of HPV invading human genitalia, anus and head and neck and for genotyping thereof. Further, it is more effective for diagnosis of cervical cancer and precancerous lesions than the existing products.

1. A DNA chip for genotyping human papillomavirus (HPV) from a sample, comprising a linear oligonucleotide probe having a base sequence selected from SEQ ID NOS 6-109.

2. A DNA chip for genotyping human papillomavirus (HPV) from a sample, comprising a d-shaped oligonucleotide probe having a base sequence selected from SEQ ID NOS 110-213.

3. The DNA chip according to claim 1, wherein the DNA chip is for simultaneously genotyping 44 types of HPV comprising: HPV-16, HPV-18, HPV-31, HPV-33, HPV-35, HPV-39, HPV-45, HPV-51, HPV-52, HPV-56, HPV-58, HPV-59, HPV-68a, HPV-68b and HPV-82 as high-risk type HPVs; HPV-26, HPV-53, HPV-66, HPV-67, HPV-69, HPV-70 and HPV-73 as moderate-risk type HPVs; HPV-6, HPV-11, HPV-34, HPV-40, HPV-42, HPV-43, HPV-44, HPV-54, HPV-55, HPV-61, HPV-62, HPV-72 and HPV-81 as low-risk type HPVs; and HPV-90, HPV-10, HPV-27, HPV-30, HPV-32, HPV-57, HPV-83, HPV-84 and HPV-91 as other HPVs.

4. The DNA chip according to claim 3, wherein the oligonucleotide probe having a base sequence selected from SEQ ID NOS 6-97 or SEQ ID NOS 110-201 binds complementarily to L1 gene region specific for each type of HPV.

5. The DNA chip according to claim 3, wherein the oligonucleotide probe having a base sequence selected from SEQ ID NOS 98-105 or SEQ ID NOS 202-209 is a universal probe binding complementarily to L1 gene region existing in all types of HPV.

6. The DNA chip according to claim 3, wherein the oligonucleotide probe having a base sequence selected from SEQ ID NOS 106-109 or SEQ ID NOS 210-213 binds complementarily to beta-actin gene as positive control.

7. The DNA chip according to claim 1, wherein the DNA chip has 8-24 partitioned wells on which the probe can be spotted.

8. The DNA chip according to claim 1, wherein the concentration of the oligonucleotide probe is at least 38 pmol.

9. The DNA chip according to claim 1, wherein C6 amine-modified dideoxythymidine is attached to the oligonucleotide probe as a linker so as to spot the oligonucleotide probe on a superaldehyde-coated support.

10. The DNA chip according to claim 9, wherein the support is selected from a group consisting of glass slide, paper, nitrocellulose membrane, microplate well, plastic, silicon, DVD and bead.

11. The DNA chip according to claim 1, wherein the sample is selected from a group consisting of cervical swab, vaginal swab, cervical tissue, penile tissue, urine, anal tissue, rectal tissue, pharyngeal tissue, oral tissue and head and neck tissue.

12. The DNA chip according to claim 1, wherein the sample is selected from a group consisting of penile cancer cell, urethral cancer cell, anal cancer cell, head and neck cancer cell and precancerous cells thereof.

13. The DNA chip according to claim 1, wherein the DNA chip is used to determine whether HPV vaccine will be administered.

14. A kit for genotyping human papillomavirus (HPV), comprising the DNA chip according to claim 1, a primer for amplifying a target gene by PCR and a label for detecting the amplified DNA.

15. The kit according to claim 14, wherein the primer is a primer for amplifying human beta-actin gene having a base sequence selected from SEQ ID NOS 1-2 or a primer for amplifying HPV L1 gene having a base sequence selected from SEQ ID NOS 3-5.

16. The kit according to claim 14, wherein the label is one or more selected from a group consisting of Cy3, Cy5, Cy5.5, BODIPY, Alexa 488, Alexa 532, Alexa 546, Alexa 568, Alexa 594, Alexa 660, rhodamine, TAMRA, FAM, FITC, Fluor X, ROX, Texas Red, Orange Green 488X, Orange Green 514X, HEX, TET, JOE, Oyster 556, Oyster 645, BODIPY 630/650, BODIPY 650/665, Calfluor Orange 546, Calfluor Red 610, Quasar 670, biotin, Au, Ag and polystyrene.

17. A method for genotyping human papillomavirus (HPV), comprising:
amplifying a target gene of a sample by single, duplex or nested PCR using a primer having a base sequence selected from SEQ ID NOS 1-5;
labeling the oligonucleotide probe of the DNA chip according to claim 1 erg;
hybridizing the labeled probe with the amplified PCR product; and
detecting the hybridized product.

18. The method according to claim 17, wherein said labeling the oligonucleotide probe comprises labeling the oligonucleotide probe with a label selected from a group consisting of Cy3, Cy5, Cy5.5, BODIPY, Alexa 488, Alexa 532, Alexa 546, Texas Red, Orange Green 488X, Orange Green 514X, HEX, TET, JOE, Oyster 556, Oyster 645, BODIPY 630/650, BODIPY 650/665, Calfluor Orange 546, Calfluor Red 610, Quasar 670 and biotin.

19. The method according to claim 17, wherein said labeling the oligonucleotide probe comprises labeling a target probe first with an Au nanoparticle and then with silver staining and binding the target probe complementarily to the oligonucleotide probe of the DNA chip.

20. The method according to claim 17, wherein said labeling the oligonucleotide probe comprises labeling a target probe first with an Au nanoparticle and then forming a silver shell and binding the target probe complementarily to the oligonucleotide probe of the DNA chip.

21. The method according to claim 19, wherein the target probe has a base sequence selected from SEQ ID NOS 214-215 and C18 linker, A10 and thiol group are sequentially attached at the 3′-terminal.

22. The method according to claim 20, wherein the target probe has a base sequence selected from SEQ ID NOS 214-215 and C18 linker, A10 and thiol group are sequentially attached at the 3′-terminal.

23. The method according to claim 17, which further comprises analyzing using plasmid vectors in which L1 genes of the 65 types of HPV described in Table 1 are inserted as positive control clones.

24. The method according to claim 17, wherein the sample is selected from a group consisting of cervical swab, vaginal swab, cervical tissue, penile tissue, urine, anal tissue, rectal tissue, pharyngeal tissue, oral tissue and head and neck tissue.

25. The method according to claim 17, wherein the sample is selected from a group consisting of penile cancer cell, urethral cancer cell, anal cancer cell, head and neck cancer cell and precancerous cells thereof.
